(19) United States
(12) Patent Application Publication
(10) Pub. No.: US 2021/0071257 A1
(43) Pub. Date: Mar. 11, 2021

(54) COMPOSITIONS AND METHODS FOR MAINTAINING SPLICING FIDELITY

(71) Applicant: President and Fellows of Harvard College, Cambridge, MA (US)

(72) Inventor: William B. Mair, Jamaica Plain, MA (US)

(21) Appl. No.: 17/023,442

(22) Filed: Sep. 17, 2020

Related U.S. Application Data
(63) Continuation of application No. 15/735,339, filed on Dec. 11, 2017, now abandoned.
(60) Provisional application No. 62/175,010, filed on Jun. 12, 2015.

(51) Int. Cl. C12Q 1/6883; A01K 67/027

(52) U.S. Cl. CPC C12Q 1/6883; A01K 67/0275; C12Q 2600/106; C12Q 2600/158; A01K 2217/05; A01K 2227/40; C12Q 2600/156

(57) ABSTRACT

The disclosure provides, among other things, compositions and methods useful for maintaining splicing fidelity in a cell. The compositions can include a compound that modulates the expression level or activity of one or more components of the spliceosome complex in a cell. In some embodiments, the compound is useful for restoring the expression level or activity of one or more splicing complex components to the expression level or activity present in the cell at an earlier chronological age. In some embodiments, the compound is useful for modulating the expression level or activity of one or more splicing complex components in the cell to the expression level or activity present in the cell under caloric restriction.

### RELATED APPLICATION

This application is a continuation U.S. application Ser. No. 15/735,339, filed Dec. 11, 2017, which is the U.S. National Stage of International Patent Application No. PCT/US2016/036917, filed Jun. 10, 2016, which claims the benefit of priority to U.S. Provisional Patent Application Ser. No. 62/175,010, filed Jun. 12, 2015, the contents of both of which are hereby incorporated by reference in their entirety.

### BACKGROUND

The global population is aging faster than cures for age-related diseases are being developed. As a result, age-onset diseases including cancer, neurodegenerative diseases, type II diabetes, cardiovascular disease, stroke, and osteoporosis are generating a public health burden, which is rapidly becoming insurmountable (9, 10). In Europe and the U.S., for example, roughly 80% of adults over 65 have one chronic disease, while approximately 50% have two or more (11). This prevalence of comorbidities in the elderly places limitations on efficacy of disease-centric approaches to therapeutics, as even dramatic advances in treatments for single pathologies will have minimal impact on the extension of disease free years in old age (12, 13).

Progress in the genetics of aging field has demonstrated that, while chronological aging is unavoidable, biological aging is malleable, and targeting cellular processes to promote homeostasis is an alternative strategy to disease based approaches to alleviate the health burdens of old age. Both environmental conditions and conserved genetic pathways strongly influence the rate of physiological aging and organisms alter the rate at which they age and succumb to disease in response to external cues. The most potent example of this is dietary restriction (DR; reduced caloric intake without malnutrition), which slows aging in every organism tested thus far and protects against multiple chronic diseases, including cancer, cardiovascular disease, and neurodegeneration.

### SUMMARY

The disclosure is based, at least in part, on the discovery that components of the spliceosome complex are required for the increased longevity of nematodes conferred by dietary restriction (DR). That is, animals, such as nematodes, under DR conditions exhibit an increased lifespan relative to animals not subjected to DR. This increased longevity is lost when spliceosome components, such as sfa-1/(hSF-1), are inhibited in the animals. The disclosure is also based, in part, on the discovery that splicing patterns change as animals age, such that splicing fidelity decreases with chronological age. This can be due, in part, to changes in the expression level or activity of one or more components of the spliceosome complex in the cells of aging animals. Accordingly, without being bound to any particular theory or mechanism of action, it is believed that RNA homeostasis is required for DR-induced longevity and that, in addition to DR, promoting splicing fidelity (and/or maintenance of a youthful spliceosome complex and/or spliceosome complex activity) will not only increase lifespan of a cell (or animal) and/or promote healthy aging, but also may prevent, delay the onset of, or lessen the severity of an age-related disorder in an animal. Because of the nexus between splicing fidelity and healthy aging, it is also believed that signatures of splicing events and/or expression/activity level of spliceosome components can be useful in diagnostic applications, such as determining the biological age of a cell or subject or determining the likelihood or risk of a subject developing an age-related disorder.

Accordingly, in one aspect, the disclosure features a method for determining the biological age of a eukaryotic cell. The method comprises, optionally, detecting a signature of splicing events in the eukaryotic cell; and determining the biological age of the eukaryotic cell by comparing the signature to one or more control signatures of defined age. The method can also include isolating nucleic acid from the eukaryotic cell. In some embodiments, the method can include obtaining the eukaryotic cell from an animal of interest.

In some embodiments of any of the methods described herein, the detecting comprises RNA-Seq technology. In some embodiments of any of the methods described herein, the detecting comprises quantitative PCR.

In some embodiments of any of the methods described herein, the signature comprises information of the presence or amount of a splicing event relative to an RNA molecule from at least five (e.g., at least six, seven, eight, nine, 10, 11, 12, 13, 14, 15, 20, 25, 30, 35, 40, 45, 50, 55, 60, 65, 70, or more than 80) genes.

In another aspect, the disclosure features a method for determining the biological age of a eukaryotic cell. The method comprises, optionally, detecting a spliceosome signature comprising information on the presence, or expression level, of at least two components of the spliceosome complex (including both protein and RNA components of the spliceosome complex) in the eukaryotic cell; and determining the biological age of the eukaryotic cell by comparing the signature to one or more control signatures of defined age. In some embodiments, the method can include isolating one or both of nucleic acid and protein from the eukaryotic cell. In some embodiments, the method can include obtaining the eukaryotic cell from an animal.

In some embodiments of any of the methods described herein, the eukaryotic cell is a cell from a nematode, a fish, a reptile, an insect, an amphibian, or a mammal. In some embodiments, the eukaryotic cell is a human cell.

In some embodiments of any of the methods described herein, the signature comprises information on the RNA expression level of two or more (e.g., at least two, three, four, five, six, seven, eight, nine, 10, 11, 12, 13, 14, 15, 20, 25, 30, 35, 40, 45, 50, 55, 60, 65, 70, 75, 80, or more than 90) components of the spliceosome. In some embodiments of any of the methods described herein, the signature comprises information on the protein expression level of two or more (e.g., at least two, three, four, five, six, seven, eight, nine, 10, 11, 12, 13, 14, 15, 20, 25, 30, 35, 40, 45, 50, 55, 60, 65, 70, 75, 80, or more than 90) components of the spliceosome. It is understood that expression level includes: (i) presence or absence of a given component of the spliceosome complex as well as (ii) the actual protein or RNA expression level or amount of a given component of the spliceosome complex.

In some embodiments of any of the methods described herein, the detecting comprises RNA-Seq technology. In some embodiments of any of the methods described herein, the detecting comprises PCR (e.g., quantitative PCR). In some embodiments of any of the methods described herein, the detecting comprises an immunoassay.

In some embodiments of any of the methods described herein, the signature comprises information of the presence and/or expression level of the sfa-1 gene or a homolog thereof. In some embodiments of any of the methods described herein, the signature comprises information of the presence and/or expression level of one or more of the sfa-1 gene, repo-1 gene, snr-2 gene, hrp-2 gene, and uaf-2 gene, or one or more of the homologs of any of the foregoing genes. In some embodiments of any of the methods described herein, the signature comprises information of the presence and/or expression level of the human homologs of the sfa-1, repo-1, snr-2, hrp-2, and uaf-2 genes. In some embodiments of any of the methods described herein, the signature comprises information of the presence and/or expression level of any one or more of the genes (e.g., nematode or human homologs) recited in Table 1.

In some embodiments of any of the methods described herein, an elevated expression level of one or more components of the spliceosome complex, relative to the expression level of the one or more components in the one or more control signatures, is indicative of the biological age of the eukaryotic cell.

In some embodiments of any of the methods described herein, a reduced expression level of one or more components of the spliceosome complex, relative to the expression level of the one or more components in the one or more control signatures, is indicative of the biological age of the eukaryotic cell.

In another aspect, the disclosure features a method for identifying one or more biomarkers of aging. The method comprises: (a) comparing: (i) a first signature of splicing events in one or more cells from a first animal, and (ii) a second signature of splicing events in one or more cells from a second animal that is chronologically older than the first animal, wherein the first animal and second animal are of the same species, and (b) identifying one or more splicing event variations between the first signature and the second signature.

In another aspect, the disclosure features a method for identifying one or more biomarkers of aging, which method includes: (a) comparing: (i) a first signature of splicing events in one or more cells from a first animal, and (ii) a second signature of splicing events in one or more cells from a second animal that has been calorically restricted, wherein the first and second animal are of the same species; and (b) identifying one or more splicing event variations between the first signature and the second signature. In some embodiments, the first animal and the second animal are substantially the same chronological age.

In some embodiments, any of the methods described herein further comprise determining one or both of the first signature and the second signature.

In yet another aspect, the disclosure features a method for identifying one or more biomarkers of aging, which method includes (a) comparing: (i) a first signature of splicing events in one or more cells from a first animal, (ii) a second signature of splicing events in one or more cells from a chronologically older animal of the same species; and a third signature of splicing events in one or more cells from a third animal that has been calorically restricted, wherein the first animal, the second animal, and the third animal are all of the same species, and (b) identifying one or more splicing event variations between the first signature and the second signature that are also splicing event variations between the first signature and the third signature. In some embodiments, the first animal and the third animal are the same chronological age. In some embodiments, the method can further comprise determining the first signature, the second signature, the third signature, the first and second signature, the first and third signature, the second and third signature, or the first, second, and third signature.

In another aspect, the disclosure features a method for determining whether a subject is at an increased risk for developing an age-related disorder. The method comprises, optionally, detecting a signature of splicing events using nucleic acid from one or more cells from the subject; and determining whether the subject is at an increased risk for developing an age-related disorder by comparing the signature to one or more control signatures of defined age.

In yet another aspect, the disclosure features a method for determining whether a subject is at an increased risk for developing an age-related disorder, which method includes detecting a spliceosome signature comprising information on the presence, or expression level, of two or more components of the spliceosome complex in the eukaryotic cell; and determining whether the subject is at an increased risk for developing an age-related disorder by comparing the signature to one or more control signatures of defined age. The age-related disorder can be, e.g., any such disorder known in the art or recited herein. For example, the age-related disorder can be a cardiovascular disease, a bone loss disorder, a neuromuscular disorder, a cancer, a tauopathy, a neurodegenerative disorder or a cognitive disorder, or a metabolic disorder. In some embodiments, the age-related disorder is sarcopenia, osteoarthritis, chronic fatigue syndrome, Alzheimer's disease, senile dementia, mild cognitive impairment due to aging, schizophrenia, Parkinson's disease, Huntington's disease, Pick's disease, Creutzfeldt-Jakob disease, stroke, CNS cerebral senility, age-related cognitive decline, pre-diabetes, diabetes, obesity, osteoporosis, coronary artery disease, cerebrovascular disease, heart attack, stroke, peripheral arterial disease, aortic valve disease, stroke, mild cognitive impairment, pre-dementia, dementia, macular degeneration, or cataracts.

Also featured is a method for determining the expected lifespan of a eukaryotic cell. The method comprises determining the expected lifespan of the eukaryotic cell by comparing a signature of splicing events in the eukaryotic cell to one or more control signatures of defined age.

In another aspect, the disclosure features a method for determining the expected lifespan of a subject, the method comprising comparing a signature of splicing events in one or more cells obtained from the subject to one or more control signatures of defined age to thereby determine the expected lifespan of the subject.

In yet another aspect, the disclosure features a method for maintaining splicing fidelity in a eukaryotic cell. The method comprises contacting the eukaryotic cell with a compound that modulates the expression level or activity of one or more components of the spliceosome to thereby maintain splicing fidelity in the eukaryotic cell, wherein the expression level or activity of the one or more components of the spliceosome is modulated to a state that mimics the expression level or activity of the one or more components of the spliceosome in the eukaryotic cell: (i) under reduced caloric intake conditions or (ii) at an earlier chronological age.

In another aspect, the disclosure features a method for prolonging the survival of a eukaryotic cell. The method comprises contacting the eukaryotic cell with a compound that modulates the expression level or activity of one or more components of the spliceosome to thereby prolong the survival of the cell.

In another aspect, the disclosure features a method for mimicking the effects of reduced caloric intake on a eukaryotic cell, which method comprises contacting the eukaryotic cell with a compound that modulates the expression level or activity of one or more components of the spliceosome to thereby mimic the effects of reduced caloric intake on the eukaryotic cell.

In some embodiments, the eukaryotic cell is a cultured cell. In some embodiments, the eukaryotic cell is in or on a multicellular organism. The cell can be from any animal known in the art or described herein. The cell can be from any organ (e.g., heart, lung, brain, colon, kidney, pancreas, bladder, skin, or spleen) or tissue (e.g., muscle, bone, marrow, or blood) of an animal known in the art or described herein.

In another aspect, the disclosure features a method for promoting healthy aging in a subject, the method comprising administering to the subject a compound that modulates the expression level or activity of one or more components of the spliceosome to thereby promote healthy aging in a subject.

In another aspect, the disclosure features a method for extending the lifespan of a subject, the method comprising administering to the subject a compound that modulates the expression level or activity of one or more components of the spliceosome to thereby extend the lifespan of the subject.

In some embodiments, the lifespan is extended by at least 2 (e.g., at least 3, 4, 5, 10, 15 20, 25, 30, 35, 40, 45, 50, 55, 60, 65, 70, 75, or 80) % relative to the lifespan of the subject or cell (or mean lifespan of a cell of the same histological type and of the same species or of subjects of the same gender, species, and health condition as the subject) in the absence of the compound.

In another aspect, the disclosure provides a method for preventing or delaying the onset of an age-related disorder in a subject, which method comprises administering to a subject in need thereof a compound that modulates the expression level or activity of one or more components of the spliceosome to thereby prevent or delay the onset of an age-related disorder in the subject.

In another aspect, the disclosure features a method for treating a subject suffering from an age-related disorder, which method comprises administering to the subject a compound that modulates the expression level or activity of one or more components of the spliceosome to thereby treat the age-related disorder in the subject. The age-related disorder can be, e.g., any such disorder known in the art or recited herein. For example, the age-related disorder can be a cardiovascular disease, a bone loss disorder, a neuromuscular disorder, a tauopathy, a neurodegenerative disorder or a cognitive disorder, a cancer, or a metabolic disorder. In some embodiments, the age-related disorder is sarcopenia, osteoarthritis, chronic fatigue syndrome, Alzheimer's disease, senile dementia, mild cognitive impairment due to aging, schizophrenia, Parkinson's disease, Huntington's disease, Pick's disease, Creutzfeldt-Jakob disease, stroke, CNS cerebral senility, age-related cognitive decline, pre-diabetes, diabetes, obesity, osteoporosis, coronary artery disease, cerebrovascular disease, heart attack, stroke, peripheral arterial disease, aortic valve disease, stroke, mild cognitive impairment, pre-dementia, dementia, macular degeneration, or cataracts.

In some embodiments of any of the methods described herein, the compound is a protein, e.g., a protein component of the spliceosome complex, such as a human homolog of sfa-1, repo-1, snr-2, hrp-2, or uaf-2 (as set forth in Table 1).

In some embodiments of any of the methods described herein, the compound is a nucleic acid (e.g., a DNA or mRNA), such as one encoding a protein component of the spliceosome complex or a nucleic acid component of the spliceosome complex. In some embodiments, the nucleic acid encodes a human homolog of sfa-1, repo-1, snr-2, hrp-2, or uaf-2, or any other human protein set forth in Table 1.

In yet another aspect, the disclosure features a transgenic non-human animal comprising a plurality of cells comprising at least three (e.g., at least four, five, six, seven, eight, nine, 10, 11, 12, 13, 14, 15, 20 or more than 20) different nucleic acids, wherein each nucleic acid encodes a different protein whose expression requires at least one specific splicing event in the cells. In some embodiments, the protein is a fluorescent protein. In some embodiments, the protein is detectably-labeled. In some embodiments, the detectable label is an epitope tag. The animal can be any animal known in the art or described herein. For example, the animal can be a nematode or a fish, such as a zebrafish.

In another aspect, the disclosure features a transgenic non-human animal cell comprising at least three different nucleic acids, wherein each nucleic acid encodes a different protein whose expression requires at least one specific splicing event in the cell. In some embodiments, the protein is a fluorescent protein. In some embodiments, the protein is detectably-labeled. In some embodiments, the detectable label is an epitope tag. The cell can be from any animal. The animal can be any animal known in the art or described herein. For example, the animal can be a nematode or a fish, such as a zebrafish.

In another aspect, the disclosure features a method to identify a compound that maintains splicing fidelity in a cell, which method comprises: contacting any one of the transgenic non-human animal cells described herein with a candidate compound; and detecting a signature of splicing events in the cell, wherein the signature comprises information of the presence, absence, or amount of the at least one specific splicing event for each of the at least different nucleic acids in the cell at a point in time after the contacting. A change in the signature in the presence of the candidate compound, as compared to the signature in the absence of the compound, indicates that the candidate compound is not a compound that maintains splicing fidelity in the cell, and wherein the lack of a significant change in the signature in the presence of the candidate compound, as compared to the signature in the absence of the compound, indicates that the candidate compound is a compound that maintains splicing fidelity in the cell.

In another aspect, the disclosure features a method to identify a compound that maintains splicing fidelity in an animal. The method comprises contacting a transgenic non-human animal described herein with a candidate compound; and detecting a signature of splicing events in cells of the animal, wherein the signature comprises information of the presence, absence, or amount of the at least one specific splicing event for each of the at least different nucleic acids in the cells at a point in time after the contacting. A change in the signature in the presence of the candidate compound, as compared to the signature in the absence of the compound, indicates that the candidate compound is not a compound that maintains splicing fidelity in the animal, and wherein the lack of a significant change in the signature in the presence of the candidate compound, as compared to the signature in the absence of the compound, indicates that the candidate compound is a compound that maintains splicing fidelity in the animal.

In yet another aspect, the disclosure features a method to identify a compound that maintains splicing fidelity in a cell. The method comprises contacting any one of the transgenic non-human animal cells described herein with a candidate compound; and detecting a signature of splicing events in the cell, wherein the signature comprises information of the presence, absence, or amount of the at least one specific splicing event for each of the at least different nucleic acids in the cell at a point in time after the contacting. A change in the signature in the presence of the candidate compound, as compared to a control signature, indicates that the candidate compound is not a compound that maintains splicing fidelity in the cell, and wherein the lack of a significant change in the signature in the presence of the candidate compound, as compared to the control signature in the absence of the compound, indicates that the candidate compound is a compound that maintains splicing fidelity in the cell.

In another aspect, the disclosure features a method to identify a compound that maintains splicing fidelity in an animal. The method includes contacting a transgenic non-human animal described herein with a candidate compound; and detecting a signature of splicing events in cells of the animal, wherein the signature comprises information of the presence, absence, or amount of the at least one specific splicing event for each of the at least different nucleic acids in the cells at a point in time after the contacting. A change in the signature in the presence of the candidate compound, as compared to a control signature, indicates that the candidate compound is not a compound that maintains splicing fidelity in the animal, and wherein the lack of a significant change in the signature in the presence of the candidate compound, as compared to the control signature, indicates that the candidate compound is a compound that maintains splicing fidelity in the animal.

In some embodiments of any of the methods described herein, the control signature is a signature of splicing events in a cell or animal of the same species under caloric restriction. In some embodiments of any of the methods described herein, the control signature is a signature of splicing events in a cell or animal of the same species at a youthful chronological age.

In some embodiments of any of the methods described herein, the point in time after the contacting is after the cell or animal has been aged, e.g., by at least 1 (e.g., at least 2, 3, 4, 5, 6, 7, 8, 9, 10, 11, 12, 13, 14, 15, 16, 17, 18, 19, 20, 24, 36, 48) hours, at least 1 (e.g., at least 2, 3, 4, 5, 6, 7, 8, 9, 10, 12, 14, 21, 28, 30, 60, or more than 60) days, at least 1 (e.g., 2, 3, 4, 5, 6, 7, 8, 9, 10, 12, 14, 21, 28, 30, 60, or more than 60) months, or at least 1 (e.g., 2, 3, 4, 5, 6, 7, 8, 9, 10, 12, 14, or 20) years.

Unless otherwise defined, all technical and scientific terms used herein have the same meaning as commonly understood by one of ordinary skill in the art to which this disclosure pertains. In case of conflict, the present document, including definitions, will control. Preferred methods and materials are described below, although methods and materials similar or equivalent to those described herein can also be used in the practice or testing of the presently disclosed methods and compositions. All publications, patent applications, patents, and other references mentioned herein are incorporated by reference in their entirety.

Other features and advantages of the present disclosure, e.g., methods for treating a subject with an age-related condition, will be apparent from the following description, the examples, and from the claims.

### BRIEF DESCRIPTION OF THE FIGURES

FIG. 1, which features five panels A-E, depicts the determination of Splicing Fidelity in vivo. Panel A. Schematic of a symmetric pair of ret-1 exon 5 reporter minigene structure. Panel B. Image of a day 1 adult KH2235 splicing reporter worm. The nervous system (N), body wall muscles (bwm) and hypodermis (hyp) predominantly express 11 E5-mCherry, while pharynx (phx) and intestine (int) predominantly express E5-EGFP. Panel C. Control animal expressing GFP and mCherry under eft-3 promoter. Panel D. Simplified schematic of the splicing machinery. Worm proteins are identified in parentheses. Panel E. Inhibition of the spliceosome by uaf-2 RNAi perturbs splicing reporter and induces heterogeneity compared to wild type (WT).

FIG. 2, which includes four panels A-D, depicts age induced defects and heterogeneity in alternative splicing. Panel A. Day 1 animals show a uniform splicing pattern that is homogeneous across individuals. Panel B. With age, heterogeneity in splicing is seen in the population such that some animals display a pattern that mimics loss of spliceosome function induced by uaf-2 RNAi. (Day 5). Panel C. By Day 7, collapse in RNAi homeostasis has occurred. Panel D. Changes seen are not due to protein half-life as fluorophores are still expressed in old ages (q RT-PCR).

FIG. 3, which includes six panels A-F, depicts DR prolongation of lifespan and maintenance of a youthful splicing pattern. Panel A. By day 10 AL fed animals show a dysfunctional splicing pattern. Panel B. Solid plate DR maintains a youthful splicing pattern. Panel C. hrp-2 RNAi induced spliceosome dysfunction that mimics old age. Panel D. Quantification of fluorescence. Panel E. DR protocol that maintains splicing fidelity in (B) robustly increases lifespan. Panel F. Replicate of DR experiment showing DR protective effect on splicing.

FIG. 4, which includes seven panels A-G, depicts the identification of Spliceosome Components Required for DR Longevity. Panel A. Schematic of splicing machinery color coded to match lifespan curves. Worm proteins are recited in parentheses. Panels B-G. Lifespan curves of N2 WT (solid) and eat-2 (dash) animals on empty vector (black) and splicing factor RNAi (Color). Panel B. Example of splicing component (RSP-3) that is not required for DR lifespan. Panels C-F RNAi of snr-1, snr-2, hrp-2 and uaf-2 shorted the lifespan of WT animals, but also completely suppress lifespan extension by DR. Panel G. RNAi of sfa-1 has no effect on WT animals, yet SFA-1 is completely required for DR longevity.

FIG. 5, which includes two panels A and B, depicts the detection of dysfunctional splicing using RNA-Seq. Panel A. hrp-2 RNAi induces a dysfunctional splicing profile in Day 1 adult worms that mimics the loss of fidelity in the reporter seen in old age. Panel B. RNA Seq (100 bp paired end reads) on was performed on WT animals with and without uaf-2 RNAi. Histogram represents fold change of transcript representation in different data sets in uaf-2 RNAi animals normalized to WT (1.0). A three-fold increase in significantly expressed exonic regions in uaf-2 RNAi animals, which contain higher frequency of exotic and aberrancy (i.e. mis-spliced) transcripts, was detected. Therefore RNA-Seq validates the above results with the reporter animals and can detect spliceosome dysfunction.

FIG. 6 provides a schematic of AL and DR RNA-Seq experiment. Time points will be determined by concurrent live lifespan analysis such that data points allow cross analysis of DR and AL RNA status at matched chronological (X axis) physiological (Y axis) ages. DR will be performed using the plate DR assay and RNAi for sfa-1 will be done from hatch until day 1 of adulthood.

FIG. 7, which includes two panels A and B, depicts the effect of sfa-1 RNAi on insulin/IGF-1 and mTOR-mediated longevity. Panel A. sfa-1 RNAi does not suppress lifespan extension seen in daf-2 (e1370) mutants. Panel B. Lifespan extension by raga-1 mutation is fully suppressed by sfa-1 RNAi.

FIG. 8 includes three panels A-C. Panels A and B depict RNAi of splicing factors prp-8 and uaf-2 (adult onset) prolong lifespan in WT nematodes. Panel C depicts expression of splicing factors (snr-2) decreases with age in C. elegans.

FIG. 9, which includes two panels A and B, depicts splicing fidelity as a predictor of lifespan. Panel A. Separation of a heterogeneous day 6 old population according to splicing efficiency. Panel B. Survival curve of both homogeneous population G and R.

FIG. 10, which includes three panels A-C, depicts the effect of sfa-1 RNAi on insulin/IGF-1 and mTOR mediated longevity. Panel A. sfa-1 RNAi fully suppresses lifespan extension seen in eat-2 (ad1116) mutants. Panel B. sfa-1 RNAi does not suppress lifespan extension seen in daf-2 (e1370) mutants. Panel C. Lifespan extension by raga-1 mutation and constitutive AMPK activity is fully suppressed by sfa-1 RNAi.

FIG. 11, which includes seven panels A-G, shows that ageing induces splicing heterogeneity. Panel A. Schematic of a pair of ret-1 exon 5 minigenes expressed in the splicing reporter strain with artificial frame shifts introduced to result in premature stop codons, preventing mCherry or EGFP expression when exon 5 is included (mCherry) or skipped (EGFP) respectively. Panel B. The nervous system, body wall muscles, and hypodermis predominantly express ΔE5-mCherry, while pharynx and intestine predominantly express E5-EGFP. differential regulation of reporter. Panel B (inset). Reporter control strain, expressing GFP and mCherry driven by ubiquitous eft-3 promoter without the minigene, show high expression of both fluorophores in all tissues. Panel C. Simplified diagram of C. elegans intron splicing showing representative splicing factors investigated herein. Panel D. Ageing induces heterogeneity in alternative splicing by day 5 of adulthood, primarily in intestinal cells, mimicking a deregulated spliceosome (indicative images shown). Panel E. By day 7 of adulthood, a collapse in RNA homeostasis has occurred and worms no longer express a youthful splicing pattern. Panel F. Representative image of a synchronized population of day 6 old adult worms separated according to youthful (increased exon inclusion, high EGFP) and aged splicing pattern (decreased EGFP expression) using light microscopy. Panel G. Animals with increased exon inclusion rate at day 6 exhibit significantly longer lifespan than worms in the increased alternative splicing population (p<0.0001, log-rank test). Arrow points to time of population sorting and start of lifespan monitoring (n=100 worms per group).

FIG. 12, which includes eight panels A-H, shows that DR maintains splicing function and splicing homeostasis is required for DR longevity. Panel A. Solid plate DR (sDR) regime robustly extends C. elegans lifespan (p<0.0001, log-rank test). Panel B. Splicing reporter worms on sDR maintain youthful splicing compared to age-matched AL fed animals and (n=8, representative image shown) Panel C. DR animals display increased exon inclusion at day 7 (p<0.0001, unpaired t-test, mean±SD, n=8). Panel D. Downregulation of Sm protein SNR-1 completely suppresses lifespan extension mediated by eat-2(ad1116) mutant (p=0.5147, log-rank), but decreases WT lifespan by 40% (p<0.0001, log-rank test). eat-2(ad1116) mutation increases WT lifespan by 55%. Panel E. Depletion of RNA binding protein HRPF-1 by RNAi has no effect on WT or DR animals (eat-2(ad1116) mutant) (p<0.0001, log-rank test). Panel F. Depleted splicing factor REPO-1 expression reduces eat-2(ad1116) longevity by 90% (p<0.0001, log-rank) without affecting WT lifespan (p=0.2010, log-rank test). Panel G. sfa-1 RNAi blocks eat-2(ad1116) mutant longevity (p=0.9783, log-rank), but does not shorten WT lifespan. Panel H. Diminished target of splicing tos-1 isoform variation with age in WT worms (day 3 versus day 15) which is abrogated in DR. sfa-1 knockdown leads to altered tos-1 isoforms ratios (n=2 biological replicates shown per condition). All lifespan data of the RNAi screen are given in Table 4.

FIG. 13, includes seven panels A-G, depicting that DR promotes splicing efficiency genome-wide. Panel A. Concurrent lifespan analysis of samples for RNA sequencing with collection time points (vertical lines, day 3, day 15 and day 27) according to physiological and chronological age WT and DR (eat-2(ad1116) animals on control and sfa-1 RNAi bacteria. Panel B. Significant increases in unannotated junction reads of total junction reads and intron inclusion reads in WT worms at day 15 relative to day3 of age (% of total reads, mean±SEM, p-value=0.0006 unannotated junction reads, p-value=0.0106, t-test after probit transformation) Panel C. Dietary restriction shows higher splicing efficiency at day 15 of adulthood compared to AL fed worms (% of total reads, mean±SEM, p-value=0.0794 unannotated junction reads, p-value=0.1189% reads in introns, t-test after probit transformation) which is lost for the intron inclusion at day 27 when DR worms match AL fed physiological age at day 15 (% of total reads at day 27 versus day 15, mean±SEM, p-value=0.084 uannotated junction reads, p-value=0.0127, % reads in introns, t-test after probit transformation) Panel D. sfa-1 RNAi reverses the beneficial effects of DR on splicing efficiency (% unannotated junction reads of total junction reads at day 15 versus day 3, mean±SEM, p-value=0.0065, t-test after probit transformation) Panel E. Heatmap of KEGG pathway analysis comparing WT AL fed worms at day 15 vs DR and DR with sfa-1 knockdown, showing sfa-1 dependent reversal of fatty acid metabolism and regulation (boxed region) Panel F. Basal and Maximal respiratory capacity is increased in DR in an SFA-1 dependent manner (**** p<0.0001, ** p<0.01, * p<0.05, unpaired t-test). Panel G. RNAi inhibition of sfa-1 suppresses CA AMPK mediated longevity.

FIG. 14, which includes nine panels A-I, depicts data that shows that DR maintains splicing homeostasis via TORC1. Panel A. aak-2 null mutants show increased splicing efficiency at day 8 of adulthood on DR comparable to WT (aak-2(ok524) p=0.5488, ns, unpaired t-test, mean±SD, n=8, 1 of 3 replicate experiments shown). Panels B and C. Exon inclusion is not significantly changed in daf-16(mu86) (p=0.1835, ns, unpaired t-test, mean±SD, n=8, 1 of 3 replicate experiments shown) mutants on DR compared to WT on DR. Panel D. raga-1(ok386) mutants exhibit reduced exon inclusion at day 8 on DR (p<0.0001, unpaired t-test, mean±SD). Panel E. raga-1(ok386) increases WT lifespan by 50% (p<0.0001, log-rank). Downregulation of SFA-1 by RNAi abolishes longevity in raga-1(ok386) mutant background (p=0.2766, log-rank test). sfa-1 RNAi in WT background does not reduce lifespan. Panel F. rsks-1(ok1255) increases WT lifespan by x % (p<0.0001, log-rank), but knockdown of sfa-1 abolishes rsks-1 mutant-mediated lifespan extension (%, p-value, log-rank test) Panel G. Immunoblots of proteins from WT MEFs serum starved for 16 h, pre-treated with rapamycin (20 nM) or Torin (250 nM) for 30 min prior to insulin stimulation (1 h, 500 nM). p-S6K T389 and P-S6 S240/S244 (markers of mTORC1 activation), total S6K, total S6 and (3-actin (loading control) are shown. To control for the SF1 antibody specificity, WT MEF transfected with non-targeting control siRNAs (siCt) or SF1 siRNA for 72 h and grown in 10% FBS, are shown on the right side of the blot. Biological duplicates are shown except for the siRNA treated lanes. Panel H. Overexpression of SFA-1 extends WT lifespan by 15% (p<0.0001, log-rank test). Panel I is a schematic model of the system discussed herein.

FIG. 15, includes two panels A and B, depicts images of inverted fluorophore ret-1 splicing minigene reporter. Panel A. An inverted minigene splicing reporter shows that splicing pattern is independent of linked fluorophore to the minigene reading frame. Panel B. Knockdown of UAF-2 disrupts homogeneous splicing reporter expression and induces splicing heterogeneity compared to WT in day 1 old adults.

FIG. 16, includes nine panels A-I, depicts heterogeneous splicing patterns in response to spliceosome dynamics. Knockdown of uaf-2 (Panel A), snr-1 (Panel B), prp-38 (Panel C), rsp-2 (Panel D), prp-8 (Panel E), unc-75 (Panel F) and uaf-1 (Panel G) by RNAi from egg hatch displays splicing heterogeneity by day 1 of adulthood. Panel H, hrp-1 depletion leads to increased splicing heterogeneity by day 4. Panel I Knockdown of sfa-1 from egg hatch leads to increased intestinal mCherry expression linked to sexon inclusion by day 3 of adulthood

FIG. 17, includes six panels A-F, shows that widespread splicing changes are detectable by RNA-Seq with hrp-2 knockdown. Depletion of hrp-2 in the ret-1 minigene splicing reporter (Panel A) as well as in the inverted fluorophore splicing reporter (Panel B) leads to increased exon 5 skipping. Panel C. Knockdown of hrp-2 strongly reduces WT lifespan by xy % (p<0.0001, log-rank test). Panel D. RNA seq coverage tracks for endogenous ret-1 confirm increased exon 5 skipping in hrp-2 knockdown samples. Panels E and F. Significant exon skipping (p=3.569e⁻¹⁴), intron inclusion (p=1.373e⁻⁹) and increased unannotated splice junctions (p=0.056) in worms with hrp-2 knockdown. Sequencing reads tracks generated by Splicing Java Coverage Viewer as part of SAJR (Ref Mazin) Height of lines represent RNA coverage of splice junctions, boxes represent intronic sequence and exonic sequence.

FIG. 18, includes two panels A and B, shows that ageing promotes increased exon skipping. Panel A. EGFP and mCherry mRNA levels up to day 8 of adulthood confirm continuous expression of the minigene with age. mCherry expression is increased with age due to increased exon skipping. Panel B, Youthful splicing worms show high exon inclusion levels, alternative splicing levels are similar in the two age-matched populations (mean±SD, n=6).

FIG. 19, includes six panels A-F, depicts data showing the effects of sfa-1 downregulation. Panel A. Pumping rates in WT and in the genetic DR model eat-2(ad1116), exhibiting reduced pumping rates, are not affected by reduced sfa-1 expression at day 1 and day 4 of adulthood. RNAi from egg hatch, n=10 worms per condition. Panel B. uaf-2 and sfa-1 are expressed in the same operon, but uaf-2 gene expression is not affected by reduced sfa-1 levels in WT worms at day 1 of adulthood with sfa-1 knocked down from egg hatch. Panel C. Age-associated isoform ratio change in a target of SFA-1, target of splicing (tos-1). Panel D. A gel electrophoresis image showing endogenous ret-1 exon 5 skipping is increased with age in WT and with sfa-1 RNAi in WT and DR worms. Panel E. A gel electrophoresis image showing assessment of ret-1 exon 5 splicing pattern in independent sample set. Panel F. The in vivo ret-1 minigene reporter shows increased mCherry expression and therefore exon 5 skipping at day 3 and day 5 of adulthood in worms with sfa-1 levels depleted.

FIG. 20, includes seven panels A-G, depicts data showing the effects of downregulating splicing factors on splicing patterns and eat-2(ad1116) lifespan. Panel A. Day 7 old eat-2(ad1116) animals exhibit increased splicing efficiency (exon inclusion) compared to the WT controls. Downregulating uaf-2 (Panel B) and snr-2 (Panel C) by RNAi strongly reduces WT and eat-2(ad1116) lifespan. Lifespans were done without the addition of FUDR. phi-9 (Panel D) and hrpf-1 (Panel E) knockdown leads to reduced exon inclusion in early adulthood in WT worms. Panel F. Knockdown of rsp-2 has no significant effect on WT or eat-2(ad1116) lifespan. Panel G. Downregulating hrp-2 strongly reduces WT and DR lifespan (xy %, p-value, log-rank test, identical WT lifespan as in FIG. 17)

FIG. 21 includes six panels A-F. Panel A. Increased heterogeneity in old WT worm populations represented by multidimension plot of significantly different splicing patterns using inclusion-ratio estimates between day 3 and day 15 old AL fed WT worms. Panel B. Significant increases in unannotated splice junctions (mean±SEM, p-value=0.0003, t-test after probit transformation) and intron inclusion events (mean±SEM, p=0.0007, t-test after probit transformation) in WT AL fed worms at day 15 of age caused by sfa-1 RNAi. Panel C. Percentage of intron reads in day 15 old DR populations with sfa-1 knockdown is increased, but exhibits high variability (mean±SEM, p-value=0.0810, t-test after probit transformation). Panel D. KEGG pathways significantly upregulated in DR worm populations at day 15 compared to WT worm populations of the same chronological age with false discovery rate of 10%. Panel E. KEGG pathways significantly upregulated in DR worm populations at day 15 compared to day 3 old DR worm populations with false discovery rate of 5%. Panel F. KEGG pathways significantly upregulated in DR worm populations with sfa-1 knockdown at day 15 compared to WT AL fed worm populations of the same chronological age with false discovery rate of 5%.

FIG. 22, includes ten panels A-J, depicts data showing RNA Seq data expression validation by quantitative RT-PCR. Assessment of gene expression levels by quantitative RT-PCR for RNA seq data validation of fat-5 (Panel A), rsr-2 (Panel B), fat-6 (Panel C), acs-2 (Panel D), fat-7 (Panel E), acs-17 (Panel F), acdh-2 (Panel G), cpr-1 (Panel H), lips-17 (Panel I) and gst-4 (Panel J) in 6 biological replicates for day 3 old WT worms and 5 biological replicates for all other comparisons day 15.

FIG. 23, includes six panels A-F, depicts the RT-PCR validation of alternative splicing events in ageing and with sfa-1 knockdown. Panel A. Sequencing reads track for lipl-7 pre-mRNA. Panel B. Increased intron inclusion between exons 4 and 5 at day 15 vs day 3 of adulthood in WT animals, but not in DR worms. Intron inclusion is increased in WT and DR worms in an sfa-1 dependent manner. Panel C. Sequencing reads track for slo-2 pre-mRNA Panel D. Increased slo-2 alternative exon y skipping in day 15 old WT and with knockdown of sfa-1 in WT and DR worms. Panel E. lea-1 mRNA exhibits upregulated exon z skipping with age and sfa-1 knockdown in WT and DR animals. Panel F. Validating slo-2 exon skipping with age in independent set of WT worms at day 1 and day 12 of adulthood. Sequencing reads tracks generated by Splicing Java Coverage Viewer as part of SAJR (Ref Mazin) Height of lines represent RNA coverage of splice junctions. Boxes represent intronic sequence and exonic sequence.

FIG. 24, includes two panels A and B, depicts data showing SF1 knockdown in Hela cells. Panel A. Knockdown of splicing factor 1 (SF1) in Hela cells leads to significantly increased unannotated junction reads and reads in introns. Panel B. KEGG pathway analysis of gene expression changes shows similar pathways downregulated upon SF1 knockdown as seen in worms population with sfa-1 knockdown. Only p value considered.

FIG. 25 includes four panels A-D. Quantification of ret-1 minigene exon inclusion (GFP intensity) in aak-2(524)(Panel A) and daf-16(mu86) (Panel B). Panel C. SFA-1 does not affect insulin/IGF signalling-mediated longevity. daf-2(e1370) mutant lifespan is 87.5% increased compared to WT in sfa-1 RNAi background (p<0.0001, log-rank). Panel D. Quantification of GFP in raga-1(ok386) mutants confirms reduced exon inclusion at day 8 on DR (p<0.0001, unpaired t-test, mean±SD)

FIG. 26 includes five panels A-E. Panel A. sfa-1 RNAi blocks RAGA-1 mediated longevity (p=0.2181, Gehan-Breslow-Wilcoxon test). Panel B. Day 1 old raga-1(ok386) adults exhibit high exon inclusion levels at the onset of DR. Panel C. Day 1 old splicing reporter worms in rsks-1(ok1255) mutant background are indifferent on WT splicing reporter worms. Panel D. Immunoblots of proteins from WT MEFs grown in 10% FBS and treated with rapamycin (16 h, 20 nM) or Torin1 (16 h, 250 nM). Immunoblotting was performed as in FIG. 14, Panel G. Biological duplicates are shown except for the siRNA treated lanes. Panel E is a plot showing smg-1 and raga-1 RNAi lifespan

FIG. 27, includes three panels A-C, shows data relating to SFA-1 overexpression. Panel A is plot showing SFA-1 overexpression leads to lifespan extension on OP50-1 bacteria. Pane B is an image showing tos-1 isoform ratios are altered in worm populations over expressing SFA-1 towards higher product isoforms in two different bacteria strains. Panel C is a bar graph showing the assessment of sfa-1 expression levels by quantitative RT-PCR shows a modest increase in expression levels.

### DETAILED DESCRIPTION

The disclosure provides, among other things, compositions and methods useful for maintaining splicing fidelity in a cell. The compositions can include a compound that modulates the expression level or activity of one or more components of the spliceosome complex in a cell. In some embodiments, the compound is useful for restoring the expression level or activity of one or more splicing complex components to the expression level or activity present in the cell at an earlier chronological age. In some embodiments, the compound is useful for modulating the expression level or activity of one or more splicing complex components in the cell to the expression level or activity present in the cell under caloric restriction. While in no way intended to be limiting, exemplary compositions, as well as applications in which those compositions are useful, are set forth below.

### Biomarkers and Diagnostic Methods

The present disclosure provides a variety of biomarkers and methods useful for determining the biological age of a eukaryotic cell, collection of eukaryotic cells (e.g., tissue or organ), and/or a subject. For instance, the methods can include analyzing a signature of splicing events in a eukaryotic cell or collection of eukaryotic cells (e.g., in a biological sample obtained from a subject of interest). The signature is compared to one or more control signatures of defined age to thereby determine the biological age of the eukaryotic cell or collection of eukaryotic cells. Alternatively, or in addition, the signature can be compared to one or more control signatures of cells under caloric restriction (e.g., cells of the same histological type as the eukaryotic cell or collection of eukaryotic cells).

As used herein, the term “chronological age” refers to the age of an animal measured by a time scale from birth (e.g., years, months, days, minutes, or seconds). By contrast, the “biological age” of an animal refers to a physiological state (e.g., the protein or RNA expression profile or splicing event signature) of a cell, collection of cells, or a tissue from the animal at a point in time, relative to the state or states occurring at earlier time(s) in the animal's lifespan.

The “splicing signature” or “signature of splicing events” comprises information of a pattern of splicing events associated with at least two (e.g., at least three, four, five, six, seven, eight, nine, 10, 11, 12, 13, 14, 15, 20, 25, 30, 35, 40, 45, 50, 55, 60, 65, 70, 75, 80, 85, or 90 or more) genes. In some embodiments, the signature can serve as a “fingerprint” for a particular cell, collection of cells, or a subject, e.g., to determine the biological age of the cell, relative to other cells or to standards or cells of known biological or chronological age. For example, the pattern of splicing for at least two genes can vary between a cell of interest and a cell of known properties, e.g., known chronological age (whether youthful or older) or subjected to dietary restriction/caloric restriction. In some embodiments, the signature can be used to determine whether one cell among several other cells comes from the same subject or tissue as the other cells.

Alternative splicing is a process by which the exons of an RNA produced by transcription of a gene (a primary gene transcript or pre-mRNA) are reconnected in multiple ways during RNA splicing. The resulting different mRNAs may be translated into different protein isoforms; thus, a single gene may code for multiple proteins (Black (2003) Ann Rev Biochem 72(1):291-336). Alternative splicing occurs as a normal phenomenon in eukaryotes, where it greatly increases the diversity of proteins that can be encoded by the genome; in humans, approximately 95% of multiexonic genes are alternatively spliced (Pan et al. (2008) Nature Genetics 40(12):1413-1415). There are numerous modes of alternative splicing observed, of which the most common is cassette exon (also called exon skipping). In this mode, a particular exon may be included in mRNAs under some conditions or in particular tissues, and omitted from the mRNA in others. There are at least five basic types of alternative splicing events (Black, supra; Matlin et al. (2005) Nature Reviews 6(5):386-398; Pan et al. (2008) Nature Genetics 40(12):1413-1415; and Sammeth et al. (2008) PLoS Comput Biol 4(8):e1000147. These include: (1) cassette exon (or exon skipping): in this case, an exon may be spliced out of the primary transcript or retained. This is the most common mode in mammalian pre-mRNAs; (2) mutually exclusive exons: one of two exons is retained in mRNAs after splicing, but not both; (3) alternative donor site: an alternative 5′ splice junction (donor site) is used, changing the 3′ boundary of the upstream exon; (4) alternative acceptor site: an alternative 3′ splice junction (acceptor site) is used, changing the 5′ boundary of the downstream exon; and (5) intron retention: a sequence may be spliced out as an intron or simply retained. This is distinguished from exon skipping because the retained sequence is not flanked by introns.

As used herein, the term “gene” is well known in the art and relates to a nucleic acid sequence which traditionally has been recognized as defining a single protein or polypeptide. Of course, alternative splicing enables the production of more than one polypeptide from a single gene. The term “gene” includes a “structural gene”, which is defined as a DNA sequence that is transcribed into RNA and translated into a protein having a specific amino acid sequence thereby giving rise to a specific polypeptide or protein.

Methods for detecting a splicing signature are known in the art and exemplified herein. For example, splicing events can be measured using polymerase chain reaction (PCR) or quantitative PCR, e.g., by a method including isolating nucleic acid (e.g., RNA) from a cell, subjecting the isolated nucleic acid to reverse transcription, subjecting the reverse transcribed nucleic acid to PCR using primer pairs for predicted exon-exon junctions from a transcript of one or more genes of interest to generate amplicons; and determining the size and/or sequence of said amplicons.

In some embodiments, the signature can be determined using RNA sequencing technology (RNA-Seq) as described in, e.g., Liu et al. (2014) BMC Bioinformatics 15(1):364; Gao et al. (2014) Tumour Biol 35(10):9585-9590; and Hu et al. (2013) Nucleic Acids Res 41(2):e39. In some embodiments, the signature can be determined using microarray technology as described in, e.g., Srinivasan et al. (2005) Methods 37:345-359. Other methods for detecting splicing events for use in preparing the signatures described herein include, without limitation, Northern blot analysis as described in, e.g., Sambrook et al., Molecular Cloning: A Laboratory Manual Second Edition vol. 1, 2 and 3. Cold Spring Harbor Laboratory Press: Cold Spring Harbor, N.Y., USA, November 1989.

In another aspect, the disclosure features a method for determining the biological age of a eukaryotic cell, the method comprising determining the biological age of the eukaryotic cell by comparing a spliceosome complex signature obtained from the cell to one or more control signatures of defined age and/or metabolic state (e.g., caloric restriction), wherein the signature comprises information on the presence, or expression level, of at least two components of the spliceosome complex in the eukaryotic cell. The method can also include detecting the spliceosome complex signature.

As used herein, the “spliceosome” is intended to refer to a ribonucleoprotein complex that removes introns from one or more pre-mRNA segments. Mammalian spliceosomes are complex structures, containing over 150 distinct proteins and five small nuclear RNAs. Thus, the term “spliceosome complex component” or like grammatical terms, as used herein, can refer to a polypeptide or protein associated with the spliceosome complex or a small nuclear RNA associated with the spliceosome complex.

In some embodiments, the component of the spliceosome complex is a protein depicted in Table 1. For example, the protein can be sfa-1, repo-1, snr-2, hrp-2, uaf-2, smu-1, snr-1, sym-2, rsp-2, uaf-1, hrp-1, hrpf-1, rsp-1, rsp-5, rsp-4, rsp-6, phi-9, asd-1, fox-1, rsp-3, prp-8, hrp-2, or smg-1. In some embodiments, the protein can be a human homolog of any of the foregoing, such as U2AF1, snRNP isoform b, SMU1, snRPD3, SF1, SF3A2, ESRP1, SRp40, U2AF65, HnRNPA1, HnRNPF, SRp75, SC35, SRSF2, SFRS3/SRp20, SNP2L1, RBM9, RBFOX2, SF2/ASF, U5 snRNP, HnRNPQ/R, or SMG-1.

Homologues of many mammalian spliceosomal proteins were found in the nematode (56, 57) and gene processing in C. elegans functions in a very similar way to mammalian and other systems (57, 58). C. elegans introns are shorter in size, lack a branch point sequence and have a shorter polypyrimidine tract. The 3′-splice site sequence is highly conserved in C. elegans. Less variation can be found in recognition of splice sites compared to the mammalian system (58). Further, it is believed that 70% of all genes in C. elegans genome are processed by trans-splicing, in which the coding sequence is assembled from two separately located RNA transcripts (59-65). The process is similar to intron splicing and utilizes the same snRNAs with their associated proteins. C. elegans has a conserved splicing machinery (Table 1) and its amenability to genetic manipulation makes it highly suited for establishing a link between RNA homeostasis and extended lifespan (41, 44, 52, 66-69).

TABLE 1

| Gene | Gene Pair Name | Human homolog |
|---|---|---|
| uaf-2 | Y116A8C.35 | U2AF1 |
| snr-2 | W08E3.1 | snRNPB |
| smu-1 | CC4.3 | SMU1 |
| snr-1 | Y116A8C.42 | snRPD3 |
| sfa-1 | Y116A8C.32 | SF1 |
| repo-1 | F11A10.2 | SF3A2 |
| sym-2 | ZK1067.6 | ESRP1 |
| rsp-2 | W02B12.2 | SRp40 |
| uaf-1 | Y92C3B.2 | U2AF65 |
| hrp-1 | F42A6.7 | HnRNPA1 |
| hrpf-1 | W02D3.11 | HnRNPF |
| rsp-1 | W02B12.3 | SRp75 |
| rsp-5 | T28D9.2 | SC35 |
| rsp-4 | EEED8.7 | SRSF2 |
| rsp-6 | C33H5.12 | SFRS3/SRp20 |
| phi-9 | M28.5 | NHP2L1 |
| asd-1 | R74.5 | RBM9 |
| fox-1 | T07D1.4 | RBFOX2 |
| rsp-3 | Y111B2A.18 | SF2/ASF |
| prp-8 | C50C3.6 | U5 snRNP |
| hrp-2 | F58D5.1 | HnRNPQ/R |

In some embodiments, determining a spliceosome signature can involve detecting or measuring the expression level or activity of one or more components of the spliceosome. Gene expression can be detected as, e.g., protein or mRNA expression of a target protein. That is, the presence or expression level (amount) of a protein can be determined by detecting and/or measuring the level of mRNA or protein expression of the protein.

A variety of suitable methods can be employed to detect and/or measure the level of mRNA expression of a protein. For example, mRNA expression can be determined using Northern blot or dot blot analysis, reverse transcriptase-PCR (RT-PCR; e.g., quantitative RT-PCR), in situ hybridization (e.g., quantitative in situ hybridization) or nucleic acid array (e.g., oligonucleotide arrays or gene chips) analysis. Details of such methods are described below and in, e.g., Sambrook et al., Molecular Cloning: A Laboratory Manual Second Edition vol. 1, 2 and 3. Cold Spring Harbor Laboratory Press: Cold Spring Harbor, N.Y., USA, November 1989; Gibson et al. (1999) Genome Res 6(10):995-1001; and Zhang et al. (2005) Environ Sci Technol 39(8):2777-2785; U.S. Patent Application Publication No. 2004086915; European Patent No. 0543942; and U.S. Pat. No. 7,101,663; the disclosures of each of which are incorporated herein by reference in their entirety.

In one example, the presence or amount of one or more discrete mRNA populations in a biological sample can be determined by isolating total mRNA from the biological sample (see, e.g., Sambrook et al. (supra) and U.S. Pat. No. 6,812,341) and subjecting the isolated mRNA to agarose gel electrophoresis to separate the mRNA by size. The size-separated mRNAs are then transferred (e.g., by diffusion) to a solid support such as a nitrocellulose membrane. The presence or amount of one or more mRNA populations in the biological sample can then be determined using one or more detectably-labeled polynucleotide probes, complementary to the mRNA sequence of interest, which bind to and thus render detectable their corresponding mRNA populations. Detectable labels include, e.g., fluorescent (e.g., fluorescein, fluorescein isothiocyanate, rhodamine, dichlorotriazinylamine fluorescein, dansyl chloride, allophycocyanin (APC), or phycoerythrin), luminescent (e.g., europium, terbium, Qdot™ nanoparticles supplied by the Quantum Dot Corporation, Palo Alto, Calif.), radiological (e.g., ¹²⁵I, ¹³¹I, ³⁵S, ³²P, ³³P, or ³H), and enzymatic (horseradish peroxidase, alkaline phosphatase, beta-galactosidase, or acetylcholinesterase) labels.

In another example, the presence or amount of discrete populations of mRNA in a biological sample can be determined using nucleic acid (or oligonucleotide) arrays (e.g., an array described below under “Arrays and Kits”). For example, isolated mRNA from a biological sample can be amplified using RT-PCR with random hexamer or oligo(dT)-primer mediated first strand synthesis. The RT-PCR step can be used to detectably-label the amplicons, or, optionally, the amplicons can be detectably labeled subsequent to the RT-PCR step. For example, the detectable label can be enzymatically (e.g., by nick translation or a kinase such as T4 polynucleotide kinase) or chemically conjugated to the amplicons using any of a variety of suitable techniques (see, e.g., Sambrook et al., supra). The detectably-labeled amplicons are then contacted to a plurality of polynucleotide probe sets, each set containing one or more of a polynucleotide (e.g., an oligonucleotide) probe specific for (and capable of binding to) a corresponding amplicon, and where the plurality contains many probe sets each corresponding to a different amplicon. Generally, the probe sets are bound to a solid support and the position of each probe set is predetermined on the solid support. The binding of a detectably-labeled amplicon to a corresponding probe of a probe set indicates the presence or amount of a target mRNA in the biological sample. Additional methods for detecting mRNA expression using nucleic acid arrays are described in, e.g., U.S. Pat. Nos. 5,445,934; 6,027,880; 6,057,100; 6,156,501; 6,261,776; and 6,576,424; the disclosures of each of which are incorporated herein by reference in their entirety.

Methods of detecting and/or for quantifying a detectable label depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

RNA can be extracted from the tissue sample by a variety of methods, e.g., the guanidium thiocyanate lysis followed by CsCl centrifugation (Chirgwin et al. 1979, Biochemistry 18:5294-5299). RNA from single cells can be obtained as described in methods for preparing cDNA libraries from single cells, such as those described in Dulac (1998) Curr Top Dev Biol 36:245 and Jena et al. (1996) J Immunol Methods 190:199. Care to avoid RNA degradation must be taken, e.g., by inclusion of RNAsin.

The RNA sample can then be enriched in particular species. In one embodiment, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In a preferred embodiment, the RNA population is enriched in marker sequences. Enrichment can be undertaken, e.g., by primer-specific cDNA synthesis, or multiple rounds of linear amplification based on cDNA synthesis and template-directed in vitro transcription (see, e.g., Wang et al. (1989) Proc Natl Acad Sci USA 86:9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by Marshall et al., (1994) PCR Methods and Applications 4: 80-84. Real time PCR may also be used.

Other known amplification methods which can be utilized herein include but are not limited to the so-called “NASBA” or “35R” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace (1989) Genomics 4: 560; Landegren et al. (1988) Science 241:1077); self-sustained sequence replication (SSR) (see, e.g., Guatelli et al. (1990) Proc Nat Acad Sci USA 87:1874); and transcription amplification (see, e.g., Kwoh et al. (1989) Proc Natl Acad Sci USA 86:1173).

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

In certain embodiments, the biological sample contains polypeptide molecules from the test subject. Alternatively, the biological sample can contain mRNA molecules from the test subject or genomic DNA molecules from the test subject.

In other embodiments, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

The expression of a protein can also be determined by detecting and/or measuring expression of a protein. Methods of determining protein expression generally involve the use of antibodies specific for the target protein of interest. For example, methods of determining protein expression include, but are not limited to, western blot or dot blot analysis, immunohistochemistry (e.g., quantitative immunohistochemistry), immunocytochemistry, enzyme-linked immunosorbent assay (ELISA), enzyme-linked immunosorbent spot (ELISPOT; Coligan et al., eds. (1995) Current Protocols in Immunology. Wiley, New York), or antibody array analysis (see, e.g., U.S. Patent Application Publication Nos. 20030013208 and 2004171068, the disclosures of each of which are incorporated herein by reference in their entirety). Further description of many of the methods above and additional methods for detecting protein expression can be found in, e.g., Sambrook et al. (supra).

In one example, the presence or amount of protein expression of a fusion can be determined using a western blotting technique. For example, a lysate can be prepared from a biological sample, or the biological sample itself, can be contacted with Laemmli buffer and subjected to sodium-dodecyl sulfate polyacrylamide gel electrophoresis (SDS-PAGE). SDS-PAGE-resolved proteins, separated by size, can then be transferred to a filter membrane (e.g., nitrocellulose) and subjected to immunoblotting techniques using a detectably-labeled antibody specific to the protein of interest. The presence or amount of bound detectably-labeled antibody indicates the presence or amount of protein in the biological sample.

In another example, an immunoassay can be used for detecting and/or measuring the expression of a protein. As above, for the purposes of detection, an immunoassay can be performed with an antibody that bears a detection moiety (e.g., a fluorescent agent or enzyme). Proteins from a biological sample can be conjugated directly to a solid-phase matrix (e.g., a multi-well assay plate, nitrocellulose, agarose, sepharose, encoded particles, or magnetic beads) or it can be conjugated to a first member of a specific binding pair (e.g., biotin or streptavidin) that attaches to a solid-phase matrix upon binding to a second member of the specific binding pair (e.g., streptavidin or biotin). Such attachment to a solid-phase matrix allows the proteins to be purified away from other interfering or irrelevant components of the biological sample prior to contact with the detection antibody and also allows for subsequent washing of unbound antibody. Here as above, the presence or amount of bound detectably-labeled antibody indicates the presence or amount of protein in the biological sample.

Methods for generating antibodies or antibody fragments specific for a protein can be generated by immunization, e.g., using an animal, or by in vitro methods such as phage display. A polypeptide that includes all or part of a target protein can be used to generate an antibody or antibody fragment. The antibody can be a monoclonal antibody or a preparation of polyclonal antibodies.

Methods for detecting or measuring gene expression can optionally be performed in formats that allow for rapid preparation, processing, and analysis of multiple samples. This can be, for example, in multi-welled assay plates (e.g., 96 wells or 386 wells) or arrays (e.g., nucleic acid chips or protein chips). Stock solutions for various reagents can be provided manually or robotically, and subsequent sample preparation (e.g., RT-PCR, labeling, or cell fixation), pipetting, diluting, mixing, distribution, washing, incubating (e.g., hybridization), sample readout, data collection (optical data) and/or analysis (computer aided image analysis) can be done robotically using commercially available analysis software, robotics, and detection instrumentation capable of detecting the signal generated from the assay. Examples of such detectors include, but are not limited to, spectrophotometers, luminometers, fluorimeters, and devices that measure radioisotope decay. Exemplary high-throughput cell-based assays (e.g., detecting the presence or level of a target protein in a cell) can utilize ArrayScan® VTI HCS Reader or KineticScan® HCS Reader technology (Cellomics Inc., Pittsburgh, Pa.).

In some embodiments, an elevated expression level of one or more components of the spliceosome complex, relative to the expression level of the one or more components in the one or more control signatures, is indicative of the biological age of the eukaryotic cell. In some embodiments, a reduced expression level of one or more components of the spliceosome complex, relative to the expression level of the one or more components in the one or more control signatures, is indicative of the biological age of the eukaryotic cell.

The term “overexpression” as used herein means an increase in the expression level of protein or nucleic acid molecule, relative to a control level. For example, a cell of interest may overexpress a protein (e.g., sfa-1) relative to another cell of the same histological type as the cell of interest. That is, e.g., a cell may overexpress one or more components of the spliceosome complex relative to a cell of the same histological type, but of a youthful chronological age. A cell may overexpress one or more components of the spliceosome complex relative to a cell of the same histological type, but that has been subjected to caloric restriction. Overexpression includes an increased expression of a given gene, relative to a control level, of at least 5 (e.g., at least 10, 15, 20, 25, 30, 35, 40, 45, 50, 55, 60, 65, 70, 75, 80, 85, 90, 95, 100, 110, 120, 130 140 150, 160 170, 180, 190, 200, or more) %. Overexpression includes an increased expression, relative to a control level, of at least 1.5 (e.g., at least 2, 2.5, 3, 4, 5, 6, 7, 8, 9, 10, 20, 30, 40, 50, 100, 1000 or more) fold.

In some embodiments, a cell may exhibit reduced expression level of one or more components of the spliceosome complex relative to a cell of the same histological type, but of a youthful chronological age. A cell may exhibit reduced expression of one or more components of the spliceosome complex relative to a cell of the same histological type, but that has been subjected to caloric restriction. Reduced expression of a given gene, relative to a control level, can be at least a 5 (e.g., at least 10, 15, 20, 25, 30, 35, 40, 45, 50, 55, 60, 65, 70, 75, 80, 85, 90, 95, or 99) % reduction in expression relative to a control value.

In some embodiments, an increase in expression of at least 10 (e.g., at least 15, 20, 25, 30, 35, 40, 45, 50, 55, 60, 65, 70, 75, 80, 85, 90, 95, 100, or more than 100)% over a control level is indicative of the biological age of the cell. In some embodiments, an increase in the expression level or activity of at least 1.5 (e.g., at least 2, 3, 4, 5, 6, 7, 8, 9, 10, 20, 30, 40) fold over a control level is indicative of the biological age of the cell.

The term “control” refers to any reference standard suitable to provide a comparison to the test sample. In some embodiments, the control is a signature (e.g., a splicing signature or spliceosome expression or activity signature) from a cell or collection of cells of the same histological type, but of a more youthful chronological age. In some embodiments, the control signature is a signature (e.g., a splicing signature or spliceosome expression or activity signature) from a cell or collection of cells of the same histological type, but subjected to dietary restriction/caloric restriction. The control signature can be determined using cells from the same individual from whom the cell or cells of interest are obtained; can be determined from cells from another individual; or determined from both cell from the same individual and/or cells from another individual.

In some embodiments, the control, e.g., control signature, can be (or can be based on), e.g., a collection of cells obtained from two or more (e.g., two, three, four, five, six, seven, eight, nine, 10, 15, 20, 25, 30, 35, or 40 or more) individuals (e.g., a mean or median level), e.g., individuals of defined chronological age and/or calorically restricted. In some embodiments, the control can be (or can be based on), e.g., one sample or a collection of cells obtained from two or more (e.g., two, three, four, five, six, seven, eight, nine, 10, 15, 20, 25, 30, 35, or 40 or more) individuals (e.g., a mean or median level) determined to have an advanced biological or chronological age. In some embodiments, the control can be (or can be based on), e.g., one sample or a collection of cells obtained from two or more (e.g., two, three, four, five, six, seven, eight, nine, 10, 15, 20, 25, 30, 35, or 40 or more) individuals (e.g., a mean or median level) determined to have a youthful biological or chronological age.

In some embodiments, the control amount is detected or measured concurrently with the test sample. In some embodiments, the control level or amount is a pre-determined range or threshold based on, e.g., average levels from a control group (e.g., cells from subjects of defined chronological age).

The methods of the present invention are not limited to use of a specific cut-point in comparing a level (e.g., spliceosome expression or activity level, or presence or degree of one or more splicing events) in the test sample to the control.

As noted above, the signatures described herein can be useful for identifying one or more biomarkers of aging. For example, (i) a first signature of splicing events in one or more cells from a first animal and (ii) a second signature of splicing events in one or more cells from a second animal that is chronologically older than the first animal (wherein the first animal and second animal are of the same species) can be compared. Any splicing event variations between the first signature and the second signature can be identified. One of more of these variations are useful biomarkers of aging.

In another aspect, (i) a first signature of splicing events in one or more cells from a first animal and (ii) a second signature of splicing events in one or more cells from a second animal that has been calorically restricted (wherein the first and second animal are of the same species) can be compared, and any splicing event variations between the first signature and the second signature can be identified. In some embodiments, the first animal and the second animal are substantially the same chronological age. One of more of these variations are useful biomarkers of aging.

In another aspect, one or more practitioners can compare: (i) a first signature of splicing events in one or more cells from a first animal, (ii) a second signature of splicing events in one or more cells from a chronologically older animal of the same species; and (iii) a third signature of splicing events in one or more cells from a third animal that has been calorically restricted, wherein the first animal, the second animal, and the third animal are all of the same species. The practitioner(s) can identify one or more splicing event variations between the first signature and the second signature that are also splicing event variations between the first signature and the third signature. Such variations are useful biomarkers of aging.

As used herein, the term “animal” refers to any mammal of the kingdom Animalia. Accordingly, the animal can be a nematode, insect (e.g., arthropod), fish (e.g., zebrafish), amphibian, bird, reptile, invertebrate, mammal (e.g., non-human mammal (e.g., a non-human primate) or a human). Mammals include, without limitation, a mouse, rat, hamster, gerbil, primate, non-human mammal, domestic animal such as dog, cat, cow, horse, goat, pig, or a human.

The signatures described herein can also be useful for determining whether a subject is at an increased risk for developing an age-related disorder. For example, a signature of splicing events developed from nucleic acid from one or more cells from the subject can be compared to one or more control signatures (e.g., signatures from one or more individuals who have an age-related disorder and/or one or more individuals who do not have an age-related disorder) of defined chronological age or associated with a defined age-related related disorder, to thereby determine whether the subject is at an increased risk for developing an age-related disorder.

Alternatively, a spliceosome signature comprising information on the presence, or expression level, of two or more components of the spliceosome complex in the eukaryotic cell can be compared to one or more control signatures (e.g., signatures from one or more individuals who have an age-related disorder and/or one or more individuals who do not have an age-related disorder) of defined chronological age or associated with a defined age-related related disorder, to thereby determine whether the subject is at an increased risk for developing an age-related disorder.

Aging is associated with many disorders, including, without limitation, cardiovascular diseases, bone loss disorders, neuromuscular disorders, neurodegenerative disorders, cognitive disorders, muscle-wasting conditions, vision disorders, and/or metabolic disorders. In some embodiments, the age-related disorder is sarcopenia, osteoarthritis, a cancer, chronic fatigue syndrome, Alzheimer's disease, senile dementia, mild cognitive impairment due to aging, schizophrenia, Parkinson's disease, Huntington's disease, Pick's disease, Creutzfeldt-Jakob disease, stroke, CNS cerebral senility, age-related cognitive decline, pre-diabetes, diabetes, obesity, osteoporosis, coronary artery disease, cerebrovascular disease, heart attack, stroke, peripheral arterial disease, aortic valve disease, stroke, mild cognitive impairment, pre-dementia, dementia, macular degeneration, or cataracts. In some embodiments, the age-related disorder is a tauopathy.

The signatures described herein can also be useful for determining the expected lifespan of a eukaryotic cell. For example, a signature of splicing events in the eukaryotic cell can be compared to one or more control signatures of defined chronological or biological age, or subject to caloric restriction/dietary restriction, to thereby determine the expected lifespan of the individual.

The signatures described herein can also be useful for determining the expected lifespan of a subject, e.g., a mammal, such as a human. For example, a signature of splicing events in one or more cells from a subject can be compared to one or more control signatures of defined chronological or biological age, or subject to caloric restriction/dietary restriction, to thereby determine the expected lifespan of the subject.

Methods for isolating cells are known in the art. For example, a biological sample, such as a biological fluid (e.g., urine, whole blood or a fraction thereof (e.g., plasma or serum), saliva, semen, sputum, cerebrospinal fluid, tears, or mucus) containing cells can be obtained. A biological sample can be further fractionated, if desired, to a fraction containing particular analytes (e.g., nucleic acids) of interest. For example, a whole blood sample can be fractionated into serum or into fractions containing particular types of proteins or nucleic acids. If desired, a biological sample can be a combination of different biological samples from a subject such as a combination of two different fluids.

Biological samples suitable for the invention may be fresh or frozen samples collected from a subject, or archival samples with known diagnosis, treatment and/or outcome history. The biological samples can be obtained from a subject, e.g., a subject having, suspected of having, or at risk of developing, an age-related disorder. Any suitable methods for obtaining the biological samples can be employed, although exemplary methods include, e.g., phlebotomy, swab (e.g., buccal swab), lavage, or fine needle aspirate biopsy procedure. Biological samples can also be obtained from bone marrow or spleen.

Methods for obtaining and/or storing samples that preserve the activity or integrity of cells in the biological sample are well known to those skilled in the art. For example, a biological sample can be further contacted with one or more additional agents such as appropriate buffers and/or inhibitors, including protease inhibitors, the agents meant to preserve or minimize changes (e.g., changes in osmolarity or pH) in protein structure. Such inhibitors include, for example, chelators such as ethylenediamine tetraacetic acid (EDTA), ethylene glycol tetraacetic acid (EGTA), protease inhibitors such as phenylmethylsulfonyl fluoride (PMSF), aprotinin, and leupeptin. Appropriate buffers and conditions for storing or otherwise manipulating whole cells are described in, e.g., Pollard and Walker (1997), “Basic Cell Culture Protocols,” volume 75 of Methods in molecular biology, Humana Press; Masters (2000) “Animal cell culture: a practical approach,” volume 232 of Practical approach series, Oxford University Press; and Jones (1996) “Human cell culture protocols,” volume 2 of Methods in molecular medicine, Humana Press.

A sample also can be processed to eliminate or minimize the presence of interfering substances. For example, a biological sample can be fractionated or purified to remove one or more materials (e.g., cells) that are not of interest. Methods of fractionating or purifying a biological sample include, but are not limited to, flow cytometry, fluorescence activated cell sorting, and sedimentation.

### Therapeutic Applications

As noted above, the disclosure also features compositions and therapeutic methods in which such compositions can be used. For example, the therapeutic methods can involve administering to a subject a compound that modulates the expression level or activity of one or more components of the spliceosome complex to thereby maintain splicing fidelity and/or a youthful spliceosome signature in a cell or cells of the subject. The compound can be, e.g., a small molecule, a nucleic acid or nucleic acid analog, a peptidomimetic, a polypeptide, a macrocycle compound, or a macromolecule that is not a nucleic acid or a protein. These compounds include, but are not limited to, small organic molecules, RNA aptamers, L-RNA aptamers, Spiegelmers, nucleobase, nucleoside, nucleotide, antisense compounds, double stranded RNA, small interfering RNA (siRNA), locked nucleic acid inhibitors, peptide nucleic acid inhibitors, and/or analogs of any of the foregoing. In some embodiments, a compound may be a protein or protein fragment.

In some embodiments, the compound inhibits a component of the spliceosome complex (e.g., prp-8 or U5 snRNP). As used herein, “inhibition” or the action of an “inhibitor” of a gene or gene product can be inhibition of: (i) the transcription of a coding sequence for one of the gene products, (ii) the translation of an mRNA encoding one of the gene products, (iii) the stability of an mRNA encoding one of the gene products, (iv) the intracellular trafficking of one of the gene products, (v) the stability of the gene products (i.e., protein stability or turnover), (vi) the interaction of the gene product with another protein, and/or (vii) the activity of one of the gene products.

As used herein, the term “inhibiting” and grammatical equivalents thereof refer to a decrease, limiting, and/or blocking of a particular action, function, or interaction. In one embodiment, the term refers to reducing the level of a given output or parameter to a quantity which is at least 10%, 15%, 20%, 25%, 30%, 35%, 40%, 45%, 50%, 55%, 60%, 65%, 70%, 75%, 80%, 85%, 90%, 95%, 99% or less than the quantity in a corresponding control. A reduced level of a given output or parameter need not, although it may, mean an absolute absence of the output or parameter. The disclosure does not require, and is not limited to, methods that wholly eliminate the output or parameter.

In some embodiments, the compound enhances a component of the spliceosome complex. As used herein, to “enhance” a gene or gene product can be enhancement of: (i) the transcription of a coding sequence for one of the gene products, (ii) the translation of an mRNA encoding one of the gene products, (iii) the stability of an mRNA encoding one of the gene products, (iv) the intracellular trafficking of one of the gene products, (v) the stability of the gene products (i.e., protein stability or turnover), (vi) the interaction of the gene product with another protein, and/or (vii) the activity of one of the gene products.

As used herein, the term “enhancing”, “promoting”, “agonizing” and grammatical equivalents thereof refer to an increase of a particular action, function, or interaction. In one embodiment, the term refers to an increase in the level of a given output or parameter to a quantity which is at least 10%, 15%, 20%, 25%, 30%, 35%, 40%, 45%, 50%, 55%, 60%, 65%, 70%, 75%, 80%, 85%, 90%, 95%, 99%, 100%, 150%, 200%, 300%, 400%, 500% or more than the quantity in a corresponding control.

As used herein, the term “interaction”, when referring to an interaction between two molecules, refers to the physical contact (e.g., binding) of the molecules with one another. Generally, such an interaction results in an activity (which produces a biological effect) of one or both of said molecules. To inhibit such an interaction results in the disruption of the activity of one or more molecules involved in the interaction.

Small Molecules and Peptides

“Small molecule” as used herein, is meant to refer to an agent, which has a molecular weight of less than about 6 kDa and most preferably less than about 2.5 kDa. Many pharmaceutical companies have extensive libraries of chemical and/or biological mixtures comprising arrays of small molecules, often fungal, bacterial, or algal extracts, which can be screened with any of the assays of the application. This application contemplates using, among other things, small chemical libraries, peptide libraries, or collections of natural products. Tan et al. described a library with over two million synthetic compounds that is compatible with miniaturized cell-based assays (J Am Chem Soc (1998) 120:8565-8566). It is within the scope of this application that such a library may be used to screen for inhibitors (e.g., kinase inhibitors) of any one of the gene products described herein, e.g., cyclin dependent kinases. There are numerous commercially available compound libraries, such as the Chembridge DIVERSet. Libraries are also available from academic investigators, such as the Diversity set from the NCI developmental therapeutics program. Rational drug design may also be employed.

Compounds useful in the methods of the present invention may be obtained from any, available source, including systematic libraries of natural and/or synthetic compounds. Compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85, which is expressly incorporated by reference); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145, which is expressly incorporated by reference).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233, each of which is expressly incorporated by reference.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl. Acad Sci USA 89:1865-1869) or on phage (Scott and Smith, 1990, Science 249:386-390; Devlin, 1990, Science 249:404-406; Cwirla et al, 1990, Proc. Natl. Acad, Sci. 87:6378-6382; Felici, 1991, J. Mol. Biol. 222:301-310; Ladner, supra., each of which is expressly incorporated by reference).

Peptidomimetics can be compounds in which at least a portion of a subject polypeptide is modified, and the three dimensional structure of the peptidomimetic remains substantially the same as that of the subject polypeptide. Peptidomimetics may be analogues of a subject polypeptide of the disclosure that are, themselves, polypeptides containing one or more substitutions or other modifications within the subject polypeptide sequence. Alternatively, at least a portion of the subject polypeptide sequence may be replaced with a non-peptide structure, such that the three-dimensional structure of the subject polypeptide is substantially retained. In other words, one, two or three amino acid residues within the subject polypeptide sequence may be replaced by a non-peptide structure. In addition, other peptide portions of the subject polypeptide may, but need not, be replaced with a non-peptide structure. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of humans or animals. It should be noted that peptidomimetics may or may not have similar two-dimensional chemical structures, but share common three-dimensional structural features and geometry. Each peptidomimetic may further have one or more unique additional binding elements.

Nucleic Acids

Nucleic acids can be used to increase expression of certain genes (see below). Alternatively nucleic acid inhibitors can be used to decrease expression of an endogenous gene encoding one of the gene products described herein. The nucleic acid antagonist can be, e.g., an siRNA, a dsRNA, a ribozyme, a triple-helix former, an aptamer, or an antisense nucleic acid. siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. The siRNA sequences can be, in some embodiments, exactly complementary to the target mRNA. dsRNAs and siRNAs in particular can be used to silence gene expression in mammalian cells (e.g., human cells). See, e.g., Clemens et al. (2000) Proc Natl Acad Sci USA 97:6499-6503; Billy et al. (2001) Proc Natl Acad Sci USA 98:14428-14433; Elbashir et al. (2001) Nature 411:494-8; Yang et al. (2002) Proc Natl Acad Sci USA 99:9942-9947, and U.S. Patent Application Publication Nos. 20030166282, 20030143204, 20040038278, and 20030224432. Antisense agents can include, for example, from about 8 to about 80 nucleobases (i.e. from about 8 to about 80 nucleotides), e.g., about 8 to about 50 nucleobases, or about 12 to about 30 nucleobases. Antisense compounds include ribozymes, external guide sequence (EGS) oligonucleotides (oligozymes), and other short catalytic RNAs or catalytic oligonucleotides which hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

siRNA molecules can be prepared by chemical synthesis, in vitro transcription, or digestion of long dsRNA by Rnase III or Dicer. These can be introduced into cells by transfection, electroporation, intracellular infection or other methods known in the art. See, for example, each of which is expressly incorporated by reference: Hannon, G J, 2002, RNA Interference, Nature 418: 244-251; Bernstein E et al., 2002, The rest is silence. RNA 7: 1509-1521; Hutvagner G et al., RNAi: Nature abhors a double-strand. Cur. Open. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052, PCT publications WO2006/066048 and WO2009/029688, U.S. published application U.S. 2009/0123426, each of which is incorporated by reference in its entirety.

Hybridization of antisense oligonucleotides with mRNA can interfere with one or more of the normal functions of mRNA. The functions of mRNA to be interfered with include all key functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA. Exemplary antisense compounds include DNA or RNA sequences that specifically hybridize to the target nucleic acid, e.g., the mRNA encoding one of the gene products described herein. The complementary region can extend for between about 8 to about 80 nucleobases, The compounds can include one or more modified nucleobases. Modified nucleobases may include, e.g., 5-substituted pyrimidines such as 5-iodouracil, 5-iodocytosine, and C₅-propynyl pyrimidines such as Cs-propynylcytosine and C₅-propynyluracil. Other suitable modified nucleobases include, e.g., 7-substituted-8-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. See, e.g., U.S. Pat. Nos. 4,987,071; 5,116,742; and U.S. Pat. No. 5,093,246; “Antisense RNA and DNA,” D. A. Melton, Ed., Cold Spring Harbor Laboratory, Cold Spring Harbor, N.Y. (1988); Haselhoff and Gerlach (1988) Nature 334:585-59; Helene, C. (1991) Anticancer Drug D 6:569-84; Helene (1992) Ann NY Acad Sci 660:27-36; and Maher (1992) Bioassays 14:807-15.

Aptamers are short oligonucleotide sequences that can be used to recognize and specifically bind almost any molecule, including cell surface proteins. The systematic evolution of ligands by exponential enrichment (SELEX) process is powerful and can be used to readily identify such aptamers. Aptamers can be made for a wide range of proteins of importance for therapy and diagnostics, such as growth factors and cell surface antigens. These oligonucleotides bind their targets with similar affinities and specificities as antibodies do (see, e.g., Ulrich (2006) Handb Exp Pharmacal 173:305-326).

Antisense or RNA interference molecules can be delivered in vitro to cells or in vivo. Typical delivery means known in the art can be used. Any mode of delivery can be used without limitation, including: intravenous, intramuscular, intraperitoneal, intraarterial, local delivery during surgery, endoscopic, or subcutaneous. Vectors can be selected for desirable properties for any particular application. Vectors can be viral, bacterial or plasmid. Adenoviral vectors are useful in this regard. Tissue-specific, cell-type specific, or otherwise regulatable promoters can be used to control the transcription of the inhibitory polynucleotide molecules. Non-viral carriers such as liposomes or nanospheres can also be used.

In the present methods, a RNA interference molecule or an RNA interference encoding oligonucleotide can be administered to the subject, for example, as naked RNA, in combination with a delivery reagent, and/or as a nucleic acid comprising sequences that express the siRNA or shRNA molecules. In some embodiments the nucleic acid comprising sequences that express the siRNA or shRNA molecules are delivered within vectors, e.g. plasmid, viral and bacterial vectors. Any nucleic acid delivery method known in the art can be used in the present invention. Suitable delivery reagents include, but are not limited to, e.g., the Minis Transit TKO lipophilic reagent; lipofectin; lipofectamine; cellfectin; polycations (e.g., polylysine), atelocollagen, nanoplexes and liposomes.

The use of atelocollagen as a delivery vehicle for nucleic acid molecules is described in Minakuchi et al. Nucleic Acids Res., 32(13):e109 (2004); Hanai et al. Ann NY Acad Sci., 1082:9-17 (2006); and Kawata et al. Mol Cancer Ther., 7(9):2904-12 (2008); each of which is incorporated herein in their entirety.

In some embodiments of the invention, liposomes are used to deliver an inhibitory oligonucleotide to a subject. Liposomes suitable for use in the invention can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the lipo some structure. In an embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference.

Opsonization inhibiting moieties suitable for modifying liposomes are preferably water-soluble polymers with a number-average molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as ganglio sides, such as ganglioside GMl. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. Preferably, the opsonization-inhibiting moiety is a PEG, PPG, or derivatives thereof. Liposomes modified with PEG or PEG-derivatives are sometimes called “PEGylated liposomes.”

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture, such as tetrahydrofuran and water in a 30:12 ratio at 60° C.

Liposomes modified with opsonization-inhibition moieties remain in the circulation much longer than unmodified liposomes. For this reason, such liposomes are sometimes called “stealth” liposomes. Stealth liposomes are known to accumulate in tissues fed by porous or “leaky” microvasculature. Thus, tissue characterized by such microvasculature defects, for example solid tumors, will efficiently accumulate these liposomes; see Gabizon, et al. (1988), Proc. Natl. Acad. Sci., USA, 18:6949-53, which is expressly incorporated by reference. In addition, the reduced uptake by the RES lowers the toxicity of stealth liposomes by preventing significant accumulation of the liposomes in the liver and spleen.

Antibodies Although antibodies are most often used to inhibit or enhance the activity of extracellular proteins (e.g., receptors and/or ligands), the use of intracellular antibodies to inhibit or enhance protein function in a cell is also known in the art (see e.g., Carlson, J. R. (1988) Mol. Cell. Biol. 8:2638-2646; Biocca, S. et al. (1990) EMBO J. 9:101-108; Werge, T. M. et al. (1990) FEBS Lett. 274:193-198; Carlson, J. R. (1993) Proc. Natl. Acad. Sci. USA 90:7427-7428; Marasco, W. A. et al. (1993) Proc. Natl. Acad. Sci. USA 90:7889-7893; Biocca, S. et al. (1994) Biotechnology (NY) 12:396-399; Chen, S-Y. et al. (1994) Hum. Gene Ther. 5:595-601; Duan, L et al. (1994) Proc. Natl. Acad. Sci. USA 91:5075-5079; Chen, S-Y. et al. (1994) Proc. Natl. Acad. Sci. USA 91:5932-5936; Beerli, R. R. et al. (1994) J. Biol. Chem. 269:23931-23936; Beerli, R. R. et al. (1994) Biochem. Biophys. Res. Commun. 204:666-672; Mhashilkar, A. M. et al. (1995) EMBO J. 14:1542-1551; Richardson, J. H. et al. (1995) Proc. Natl. Acad. Sci. USA 92:3137-3141; PCT Publication No. WO 94/02610 by Marasco et al.; and PCT Publication No. WO 95/03832 by Duan et al., each of which is expressly incorporated by reference). Therefore, antibodies specific for any of the gene products described herein are useful as biological agents for the methods of the present invention.

Spliceosome Complex Proteins and Nucleic Acids Encoding the Proteins

In some embodiments, a nucleic acid encoding one or more spliceosome components is administered to a subject or introduced into a cell. Nucleic acids encoding a therapeutic polypeptide can be incorporated into a gene construct to be used as a part of a gene therapy protocol to deliver nucleic acids that can be used to express and produce agents within cells. Expression constructs of such components may be administered in any therapeutically effective carrier, e.g. any formulation or composition capable of effectively delivering the component gene to cells in vivo. Approaches include insertion of the subject gene in viral vectors including recombinant retroviruses, adenovirus, adeno-associated virus, lentivirus, and herpes simplex virus-1 (HSV-1), or recombinant bacterial or eukaryotic plasmids. Viral vectors can transfect cells directly; plasmid DNA can be delivered with the help of, for example, cationic liposomes (lipofectin) or derivatized, polylysine conjugates, gramicidin S, artificial viral envelopes or other such intracellular carriers, as well as direct injection of the gene construct or CaPO₄ precipitation (see, e.g., WO04/060407) carried out in vivo. Examples of suitable retroviruses include pLJ, pZIP, pWE and pEM which are known to those skilled in the art (see, e.g., Eglitis et al. (1985) Science 230:1395-1398; Danos and Mulligan (1988) Proc Natl Acad Sci USA 85:6460-6464; Wilson et al. (1988) Proc Natl Acad Sci USA 85:3014-3018; Armentano et al. (1990) Proc. Natl. Acad. Sci. USA 87:6141-6145; Huber et al. (1991) Proc Natl Acad Sci USA 88:8039-8043; Ferry et al. (1991) Proc Natl Acad Sci USA 88:8377-8381; Chowdhury et al. (1991) Science 254:1802-1805; van Beusechem et al. (1992) Proc Natl Acad Sci USA 89:7640-7644; Kay et al. (1992) Human Gene Therapy 3:641-647; Dai et al. (1992) Proc Natl Acad Sci USA 89:10892-10895; Hwu et al. (1993) J Immunol 150:4104-4115; U.S. Pat. Nos. 4,868,116 and 4,980,286; PCT Publication Nos. WO89/07136, WO89/02468, WO89/05345, and WO92/07573). Another viral gene delivery system utilizes adenovirus-derived vectors (see, e.g., Berkner et al. (1988) BioTechniques 6:616; Rosenfeld et al. (1991) Science 252:431-434; and Rosenfeld et al. (1992) Cell 68:143-155). Suitable adenoviral vectors derived from the adenovirus strain Ad type 5 d1324 or other strains of adenovirus (e.g., Ad2, Ad3, Ad7, etc.) are known to those skilled in the art. Yet another viral vector system useful for delivery of the subject gene is the adeno-associated virus (AAV). See, e.g., Flotte et al. (1992) Am J Respir Cell Mol Biol 7:349-356; Samulski et al. (1989) J Virol 63:3822-3828; and McLaughlin et al. (1989) J Virol 62:1963-1973. In some embodiments, the nucleic acid is an mRNA or modified mRNA that encodes one or more components of the spliceosome complex, see, e.g., Zangi et al. (2013) Nature Biotechnol 31:898-907.

In some embodiments, a polypeptide (e.g., a spliceosome complex component) is administered to a subject or introduced into a cell.

Formulations

The compositions described herein can be formulated as a pharmaceutical solution, e.g., for administration to a subject treating an age-related disorder or promoting healthy aging in an individual. The pharmaceutical compositions will generally include a pharmaceutically acceptable carrier. As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see e.g., Berge et al. (1977) J Pharm Sci 66:1-19).

The compositions can be formulated according to standard methods. Pharmaceutical formulation is a well-established art, and is further described in, e.g., Gennaro (2000) “Remington: The Science and Practice of Pharmacy,” 20^{th} Edition, Lippincott, Williams & Wilkins (ISBN: 0683306472); Ansel et al. (1999) “Pharmaceutical Dosage Forms and Drug Delivery Systems,” 7^{th} Edition, Lippincott Williams & Wilkins Publishers (ISBN: 0683305727); and Kibbe (2000) “Handbook of Pharmaceutical Excipients American Pharmaceutical Association,” 3^{rd} Edition (ISBN: 091733096X). In some embodiments, a composition can be formulated, for example, as a buffered solution at a suitable concentration and suitable for storage at 2-8° C. (e.g., 4° C.). In some embodiments, a composition can be formulated for storage at a temperature below 0° C. (e.g., −20° C. or −80° C.). In some embodiments, the composition can be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, in some embodiments, the compositions described herein are stable in storage for at least 1 year at 2-8° C. (e.g., 4° C.).

The pharmaceutical compositions can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application. For example, compositions containing a composition intended for systemic or local delivery can be in the form of injectable or infusible solutions. Accordingly, the compositions can be formulated for administration by a parenteral mode (e.g., intravenous, subcutaneous, intraperitoneal, or intramuscular injection). “Parenteral administration,” “administered parenterally,” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion (see below).

The compositions can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filtered sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

The compositions described herein can also be formulated in immunoliposome compositions. Such formulations can be prepared by methods known in the art such as, e.g., the methods described in Epstein et al. (1985) Proc Natl Acad Sci USA 82:3688; Hwang et al. (1980) Proc Natl Acad Sci USA 77:4030; and U.S. Pat. Nos. 4,485,045 and 4,544,545. Liposomes with enhanced circulation time are disclosed in, e.g., U.S. Pat. No. 5,013,556.

In certain embodiments, compositions can be formulated with a carrier that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art. See, e.g., J. R. Robinson (1978) “Sustained and Controlled Release Drug Delivery Systems,” Marcel Dekker, Inc., New York.

In some embodiments, compositions described herein are administered in an aqueous solution by parenteral injection. The disclosure features pharmaceutical compositions comprising an effective amount of the agent (or more than one agent) and optionally include pharmaceutically acceptable diluents, preservatives, solubilizers, emulsifiers, adjuvants and/or carriers. Such compositions include sterile water, buffered saline (e.g., Tris-HCl, acetate, phosphate), pH and ionic strength; and optionally, additives such as detergents and solubilizing agents (e.g., TWEEN® 20, TWEEN 80, Polysorbate 80), anti-oxidants (e.g., ascorbic acid, sodium metabisulfite), and preservatives (e.g., thimersol, benzyl alcohol) and bulking substances (e.g., lactose, mannitol). The formulations may be sterilized, e.g., using filtration, incorporating sterilizing agents into the compositions, by irradiating the compositions, or by heating the compositions.

As described above, relatively high concentration compositions can be made. For example, the compositions can be formulated at a concentration of the active agent of between about 10 mg/mL to 100 mg/mL (e.g., between about 9 mg/mL and 90 mg/mL; between about 9 mg/mL and 50 mg/mL; between about 10 mg/mL and 50 mg/mL; between about 15 mg/mL and 50 mg/mL; between about 15 mg/mL and 110 mg/mL; between about 15 mg/mL and 100 mg/mL; between about 20 mg/mL and 100 mg/mL; between about 20 mg/mL and 80 mg/mL; between about 25 mg/mL and 100 mg/mL; between about 25 mg/mL and 85 mg/mL; between about 20 mg/mL and 50 mg/mL; between about 25 mg/mL and 50 mg/mL; between about 30 mg/mL and 100 mg/mL; between about 30 mg/mL and 50 mg/mL; between about 40 mg/mL and 100 mg/mL; between about 50 mg/mL and 100 mg/mL; or between about 20 mg/mL and 50 mg/mL). In some embodiments, compositions can be formulated at a concentration of greater than 5 mg/mL and less than 50 mg/mL. Methods for formulating a protein in an aqueous solution are known in the art and are described in, e.g., U.S. Pat. No. 7,390,786; McNally and Hastedt (2007), “Protein Formulation and Delivery,” Second Edition, Drugs and the Pharmaceutical Sciences, Volume 175, CRC Press; and Banga (1995), “Therapeutic peptides and proteins: formulation, processing, and delivery systems,” CRC Press. In some embodiments, the aqueous solution has a neutral pH, e.g., a pH between, e.g., 6.5 and 8 (e.g., between and inclusive of 7 and 8). In some embodiments, the aqueous solution has a pH of about 6.6, 6.7, 6.8, 6.9, 7, 7.1, 7.2, 7.3, 7.4, 7.5, 7.6, 7.7, 7.8, 7.9, or 8.0. In some embodiments, the aqueous solution has a pH of greater than (or equal to) 6 (e.g., greater than or equal to 6.1, 6.2, 6.3, 6.4, 6.5, 6.6, 6.7, 6.8, 6.9, 7, 7.1, 7.2, 7.3, 7.4, 7.5, 7.6, 7.7, 7.8, or 7.9), but less than pH 8.

Methods

In some embodiments, the methods include receiving a splicing signature or the results of a test determining that based on a signature described herein, the subject is in need to treatment (e.g., to promote healthy aging or treat, prevent, or delay the onset of an age-related disorder). Age-related disorders are well known in the art and include those recited herein. And, in view of this information, ordering administration of an effective amount of a compound that modulates the expression or activity of one or more components of the spliceosome complex to the subject. For example, a physician treating a subject can request that a third party (e.g., a CLIA-certified laboratory) to perform a test to determine a splicing signature, the biological age of a subject, and/or information indicative of the expected lifespan of a subject and/or likelihood of developing an age-related disorder. The laboratory may provide such information, or, in some embodiments, provide a score, value, or information on the status on one or more splicing events of interest. The physician may then administer to the subject a modulator of one or more components of the spliceosome to thereby maintain splicing fidelity and/or restore splicing fidelity in the subject. Alternatively, the physician may order the administration of the modulator to the subject, which administration is performed by another medical professional, e.g., a nurse.

When compositions are to be used in combination with a second active agent, the compositions can be coformulated with the second agent or the compositions can be formulated separately from the second agent formulation. For example, the respective pharmaceutical compositions can be mixed, e.g., just prior to administration, and administered together or can be administered separately, e.g., at the same or different times (see below).

The compositions described herein can be administered to a subject, e.g., a human subject, using a variety of methods that depend, in part, on the route of administration. The route can be, e.g., intravenous injection or infusion (IV), subcutaneous injection (SC), intraperitoneal (IP) injection, or intramuscular injection (IM).

Administration can be achieved by, e.g., local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; EP488401; and EP 430539, the disclosures of each of which are incorporated herein by reference in their entirety. The composition can be delivered to the subject by way of an implantable device based on, e.g., diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

As used herein the term “effective amount” or “therapeutically effective amount”, in an ire vivo setting, means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease. and the treatment being effected.

Suitable human doses of any of the compounds described herein can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

Toxicity and therapeutic efficacy of such compositions can be determined by known pharmaceutical procedures in cell cultures or experimental animals (e.g., animal models of cancer). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Agents that exhibits a high therapeutic index are preferred. While compositions that exhibit toxic side effects may be used, care should be taken to design a delivery system that targets such compounds to the site of affected tissue and to minimize potential damage to normal cells and, thereby, reduce side effects.

The data obtained from the cell culture assays and animal studies can be used in formulating a range of dosage for use in humans. The dosage of such antibodies or antigen-binding fragments thereof lies generally within a range of circulating concentrations of the antibodies or fragments that include the ED₅₀ with little or no toxicity. The dosage may vary within this range depending upon the dosage form employed and the route of administration utilized. A therapeutically effective dose can be estimated initially from cell culture assays. A dose can be formulated in animal models to achieve a circulating plasma concentration range that includes the IC₅₀ (i.e., the concentration of the antibody which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration is desired, cell culture or animal modeling can be used to determine a dose required to achieve a therapeutically effective concentration within the local site.

### Screening Methods

The disclosure also features transgenic animal cells, as well as transgenic, non-human animals. Such cells and animals are useful in a variety of screening methods for, among other things, identifying genes involved in aging and/or compounds that maintain or restore splicing fidelity (or youthful splicing) in a cell. For example, the disclosure features a transgenic non-human animal comprising a plurality of cells, each of the cells comprising at least two (e.g., at least three, four, five, six, seven, eight, nine, 10, 11, 12, 15, or 20 or more) different nucleic acids, wherein each nucleic acid encodes a different protein whose expression requires at least one specific splicing event in the cells. As exemplified in the working examples, the nucleic acids can optionally include a detectable protein (e.g., a fluorescent protein) whose detection depends on a particular splicing event giving rise to the protein. In some embodiments, the length or size of a protein is an indicator of the splicing event. In some embodiments, the presence or absence of the protein, or an mRNA encoding the protein, is an indicator of the splicing event. Methods for detecting splicing events (e.g., visually (e.g., using fluorescence), RNA-Seq, PCR, and the like) are described herein and exemplified in the working examples.

As noted above, the transgenic animal can be any animal other than a human. In some embodiments, the animal is a nematode (e.g., C. elegans). In some embodiments, the animal is a fish (e.g., a zebrafish). In some embodiments, the animal is an insect (e.g., D. melanogaster). In some embodiments, the animal is one that is amenable to high throughput screening.

Methods for producing transgenic animals are known in the art. For example, methods for producing transgenic nematodes are described in, e.g., U.S. Patent Application Publication No. 20080168573 and International Patent Application Publication Nos. WO 1998/02897 and WO 1999/992652, and are exemplified herein. Methods for making transgenic fish are described in, e.g., International Patent Application Publication No. WO 2002/082043 and U.S. Patent Application Publication Nos. 2004/0117866 and 20090255006. Suitable methods for producing transgenic animals are also reviewed in Houdebine (2002) J Biotechnol 98:145-160.

In some embodiments, the disclosure features a method to identify a compound that maintains splicing fidelity in a cell. The method comprises contacting the transgenic non-human animal cell with a candidate compound; and detecting a signature of splicing events in the cell, wherein the signature comprises information of the presence, absence, or amount of the at least one specific splicing event for each of the at least different nucleic acids in the cell at a point in time after the contacting. A change in the signature in the presence of the candidate compound, as compared to the signature in the absence of the compound, indicates that the candidate compound is not a compound that maintains splicing fidelity in the cell, and wherein the lack of a significant change in the signature in the presence of the candidate compound, as compared to the signature in the absence of the compound, indicates that the candidate compound is a compound that maintains splicing fidelity in the cell.

As used herein, a “significant change” is a change of at least 5 (e.g., at least 6, 7, 8, 9, 10, 15, 20, 25, 30, 35, 40, 45, 50, 55, 60, 65, 70, 75, 80, 85, 90, or 95 or more) %.

The disclosure also features a method to identify a compound that maintains splicing fidelity in an animal. The method includes administering to a transgenic non-human animal described herein with a candidate compound; and detecting a signature of splicing events in cells of the animal, wherein the signature comprises information of the presence, absence, or amount of the at least one specific splicing event for each of the at least different nucleic acids in the cells at a point in time after the contacting. A change in the signature in the presence of the candidate compound, as compared to the signature in the absence of the compound, indicates that the candidate compound is not a compound that maintains splicing fidelity in the animal, and wherein the lack of a significant change in the signature in the presence of the candidate compound, as compared to the signature in the absence of the compound, indicates that the candidate compound is a compound that maintains splicing fidelity in the animal.

The disclosure also features a method to identify a compound that maintains splicing fidelity in a cell. The method can include contacting a transgenic non-human animal cell described herein with a candidate compound; and detecting a signature of splicing events in the cell, wherein the signature comprises information of the presence, absence, or amount of the at least one specific splicing event for each of the at least different nucleic acids in the cell at a point in time after the contacting. A change in the signature in the presence of the candidate compound, as compared to a control signature, indicates that the candidate compound is not a compound that maintains splicing fidelity in the cell, and wherein the lack of a significant change in the signature in the presence of the candidate compound, as compared to the control signature in the absence of the compound, indicates that the candidate compound is a compound that maintains splicing fidelity in the cell.

In addition, the disclosure features a method to identify a compound that maintains splicing fidelity in an animal. The method includes administering to a transgenic non-human animal described herein with a candidate compound; and detecting a signature of splicing events in cells of the animal, wherein the signature comprises information of the presence, absence, or amount of the at least one specific splicing event for each of the at least different nucleic acids in the cells at a point in time after the contacting. A change in the signature in the presence of the candidate compound, as compared to a control signature, indicates that the candidate compound is not a compound that maintains splicing fidelity in the animal, and wherein the lack of a significant change in the signature in the presence of the candidate compound, as compared to the control signature, indicates that the candidate compound is a compound that maintains splicing fidelity in the animal.

As noted above, the control signature can be a signature of splicing events in a cell or animal of the same species under caloric restriction. Alternatively, or in addition, the control signature can be a signature of splicing events in a cell or animal of the same species at a youthful chronological age. Such screening methods are described herein and exemplified in the following working examples.

The following examples are meant to illustrate, not to limit, the disclosure.

### EXAMPLES

### Example 1: General Methods

Worm strains and culture—The following C. elegans strains were obtained from the Caenorhabditis Genetic Center, funded by the NIH Office of Research Infrastructure Programs (P40 OD010440): N2 Bristol wild type, DA1116 (eat-2(ad1116)II), RB754 (aak-2(ok524)X), VC222 (raga-1(ok386)II), and RB1206 (rsks-1(ok1255)III), (three latter strains made by groups part of the International C. elegans Knockout Consortium). The generation of the CA AMPK strain was previously described (92, 93). CF1038 (daf-16(mu86)I) and CF1041 (daf-2(e1370)III) were obtained from the Kenyon lab via the Dillin lab. The SS104 (glp-4(bn2)) strain was a gift from K. Blackwell lab. The splicing reporter strain KH2235 (lin-15(n765)ybIs2167[eft-3::ret-1E4E5(+1)E6-GGS6-mCherry+eft-3::ret-1E4E5(+1)E6(+2)GGS6-GFP+lin-15(+)+pRG5271Neo]X) was a gift from Hidehito Kuroyanagi. The inverted splicing reporter WBM535 was made by injecting modified minigene reporter plasmids into N2 worms. The plasmids were made by deleting the (+2) frameshift in the EGFP minigene and inserting a (+2) frameshift into the mCherry minigene. Worms were grown and maintained on standard nematode growth media (NGM) seeded with E. coli (OP50-1). E. coli bacteria were cultured overnight in LB at 37° C., after which 100 μl of liquid culture was seeded on plates to grow for 2 days at room temperature.

Lifespans—All lifespans were conducted at 20° C. unless otherwise noted in figure legend. Lifespans were performed as described in Burkewitz et al. (93). Graphpad Prism 6 was used to plot survival curves and determine median lifespan. Survival curves were compared and p values calculated using the log-rank (Mantel-Cox) analysis method. Complete lifespan data are available in Supplementary Information.

RNA interference—All RNAi constructs came from the Ahringer RNAi library, except hrp-2 and hrpf-1 RNAi constructs, which originated from the Vidal RNAi library. RNAi experiments were carried out using E. coli HT115 bacteria on standard NGM plates containing 100 μg/ml Carbenicillin. HT115 bacteria expressing RNAi constructs were grown overnight in LB supplemented with 100 μg/ml Carbenicillin and 12.5 μg/ml Tetracycline. NGM plus Carbenicillin plates were seeded 48 hours before use. Respective dsRNA expressing HT115 bacteria were induced by adding 100 μl IPTG (100 mM) one hour before introducing worms to the plate. RNAi was induced for all experiments from egg hatch.

Solid plate-based dietary restriction assays—Solid sDR assays were performed as described by Ching et al. (79). Plates were prepared in advance and stored at 4° C. 5-Fluoro-2′-deoxyuridine (FUDR) was added on top of the bacterial lawn (100 μl of 1 mg/ml solution in M9) 24 hours before worms were introduced to the plates for lifespans or directly into the NGM at 25 μM concentration for imaging experiments. Ad libitum (AL) plates were prepared with a bacterial concentration of 10¹¹ cfu/ml and dietary restriction plates with 10⁸ cfu/ml bacterial concentration.

Imaging—Worms were anaesthetized in 0.1 mg/ml tetramisole/M9 on an NGM plate without bacteria until no movement was detectable, aligned to groups accordingly and subsequently imaged on a Zeiss Discovery V8 microscope with Axiocam camera. Exposure times were kept constant for all imaging experiments involving the splicing reporter strain KH2235. Representative images were processed with ImageJ. Pixel intensity was determined per worm for EGFP and mCherry and background fluorescence subtracted. Results were graphed using Prism 6.

tos-1 RT-PCR constructs—tos-1 was amplified using modified PCR conditions and full-length tos-1 cDNA primers according to Ma el al. (115). Expand High Fidelity PCR System (Roche) was used for amplification with an annealing temperature of 60° C. and 35 cycles. tos-1 RT-PCR was done with 3 replicate samples of day 1 and day 12 old worm populations.

RNA extraction—Total RNA was extracted using Qiazol reagent (QIAGEN), column purified by RNeasy mini or miRNeasy micro kit (QIAGEN) according to manufacturer's instructions. cDNA was synthesized using SuperScript® VILO Master mix (Invitrogen).

Ageing and DR RNA Sequencing sample preparation—bleached to HT115, egg lay, eat-2 12 hours ahead of N2 to make up for developmental delay, on ev and sfa-1 IPTG induced bacteria, Put on FUDR at day 1, transfer at day 3+collecting first samples,

RNA Sequence analysis—Raw reads were adapter trimmed with cutadapt (ref) using the additional parameters “- -trim-n -m 15” and subsequently aligned to WBcel235 and hg38 genomes with STAR (ref) version 2.5.0c using the additional parameter “- -alignIntronMax 50000” for the WBcel235 alignments and the additional parameters “- -outSAMstrandField intronMotif - -outFilterType BySJout” for all alignments. Gene counts were obtained with htseq-count and WBcel235 Ensembl annotation v75 and hg38 Ensembl annotation v79. Gene expression analysis was performed using DESeq2—with cqn based normalization. After adjusting for multiple testing using Benjamini-Hochberg, differentially expressed genes were defined as those with adjusted p-values below 0.1. GO enrichment analysis was carried out using goseq and KEGG pathway analysis with gage, significant pathways and GO terms were defined as having Benjamini-Hochberg adjusted p-values below 0.1. Splicing analysis was done with SAJR by first constructing de novo annotation from the Ensembl input and merged alignment files. Inclusion and exclusion reads were subsequently obtained for each splicing event and analyzed using a quasibinomial model. Before model parameter estimation the analysis was restricted to only those splicing events for which at least 10 inclusion reads and 10 exclusion reads across conditions could be mapped. Significant splicing changes were defined as those with p-values below 0.05 after adjusting for multiple testing using Benjamini-Hochberg correction. PTC containing regions were defined as those containing a stop-codon in all possible reading frames at least 50 bp upstream of the next downstream splicing site.

Quantitative RT-PCR of RNA Seq sample validation—Taqman real-time qPCR experiments were performed on a StepOne Plus instrument (Applied Biosystems) following the manufacturer's instructions. Data were analysed with the comparative 2ΔΔCt method using Y45F10D.4 (Ce02467253_g1) as endogenous control. For each gene in each mutant strain, average fold-change relative to the wild type was calculated and statistical significance evaluated with a one-way analysis of variance (ANOVA). The following Taqman assays from Life Technologies were used: sfa-1 (Ce02468921_ml), uaf-2 (custom made), rsr-2 (Ce02439948_g1), cpr-1 (Ce02482188_g1), acs-2 (Ce02486192_g1), acs-17 (Ce02495808_g1), fat-5 (Ce02488494_m1), (Ce02465318_g1), fat-7 (Ce02477067_g1), acdh-2 (Ce02432818_g1), lips-17 (Ce02435133_g1) and gst-4 (Ce02458730_g1).

EGFP and mCherry expression analysis was performed using Fast SYBR Green Master Mix (Applied Biosystems) on a StepOne Plus instrument (Applied Biosystems) according to manufacturer's instructions. A standard curve was prepared to analyse EGFP and mCherry primer efficiencies. Data were analysed with the comparative 2ΔΔCt method using Y45F10D.4 and pmp-3 mRNA levels as endogenous controls. Graphpad Prism 6 was used for all statistical analysis.

Splicing validation RT-PCR—Alternative splicing events detected by our RNA seq analysis were validated by RT-PCR.

hrp-2 knockdown RNA Sequencing—The temperature-sensitive sterile glp-4(bn2) mutant strain was used for RNA sequencing and the experiment was performed with three biological replicates. Worms were grown to gravid adults and bleached to collect staged eggs. Eggs were pipetted on to IPTG-induced NGM plus Carbenicillin plates prepared with either empty vector (ev) HT115 or hrp-2 dsRNA expressing bacteria. Worms were grown at 15° C. for 24 hours, then shifted to 22.5° C. to prevent normal proliferation of germ cells and progeny development. Day 1 adult worms were washed off ev and hrp-2 plates in M9 following snap freeze in Qiazol (Qiagen) for RNA extraction. RNA extraction was performed in parallel for two replicates with a third replicate added later to obtain similar RNA levels. RNA extractions were done according to above description for qRT-PCR. RNA quality was confirmed on Agilent 2100 Bioanalyzer and all samples had RIN>8.6. cDNA libraries were prepared from 1 μg total RNA using TruSeq RNA Sample preparation v2 kit (Illumina). 100-cycle paired-end sequencing was performed on HiSeq 2500 (Illumina) by the Tufts University Core Facility.

Hela cell culture, SF1 knockdown, RNA sequencing sample preparation—HeLa cells were grown in RPMI1640 supplemented with 10% fetal calf serum (FCS), glutamine and penicillin/streptomycin. The cells were reverse transfected using RNAiMAX (ThermoFisher Scientific) and siRNAs targeting SF1 (L-012662-01-0020, Dharmacon) or non-targeting siRNAs (D-001810-10-20) as control. After 24 hours, cells were re-transfected using the forward transfection protocol and 48 hours later harvested for RNA and protein. Knockdown of SF1 was validated by Western blotting. Total RNA was isolated using Isol-RNA lysis reagent (5 PRIME). RNA purity, integrity and concentration were determined using an Agilent 2100 Bioanalyzer (Agilent Technologies, Inc, USA). Only RNA samples with a RIN value of 8.0 or higher and a ratio (28s/18s) of above 1.8 were used in sequencing library preparation. Samples were processed for library constructions following the manufactory instructions (I lumina Tru Seq Stranded Total RNA sample preparation v2 Guide, Part #15031048 Rev.E October 2013—“Low sample protocol”). In brief, 0.5 μg of total RNA from each tissue was depleted for cytoplasmic rRNA using the Ribo-Zero ribosomal reduction chemistry, chemically fragmented for 8 min at 94° C., and processed for 1st strand synthesis cDNA and then 2nd strand synthesis using dUTP instead of dTTP. After some purification steps, the cDNA was then end-repaired, purified, adenylated at the 3′-ends, and purified before adding the indexed adapter sequences using the TruSeq Stranded LT Kit Index set A. Each library preparation was then enriched by 10 cycles of PCR, purified and finally validated in regards to size and concentration. For sizing, libraries were analyzed on the Agilent 2100 Bioanalyzer using a DNA 1000 kit from Agilent Technologies. The Libraries were quantified by qPCR using the KaPa Library quantification Kits (KaPa Biosystems, Cat KK4824). Samples were pooled in sets of 12 libraries, and a final concentration of 16 pM denatured library was used for 100-cycle paired-end sequencing using an Illumina HiSeq1500 at the University of Southern Denmark's Villum Center for Bioanalytical Sciences. Antibodies—Antibodies toward SF1 (Cat no: HPA018883-100UL) and (3-actin were from Sigma. phospho (P)-S6K1 T389 (CST #9234), phospho (P)-S6 S240/S244 (CST #2215), S6K1 (CST #2708), S6 (CST #2217 were from Cell Signaling technologies.

siRNA—siRNA against mouse SF1 was from Sigma (Cat no: SASI_Mm02_00305738) and non-targeting control siRNA was from Dharmacon.

Cell culture conditions—WT-MEFs were maintained in Dulbecco's Modified Eagle's Medium (DMEM; Corning/Cellgro, 10-017-CV) containing 10% fetal bovine serum (FBS).

### Example 2. Assaying Splicing Fidelity In Vivo in C. elegans

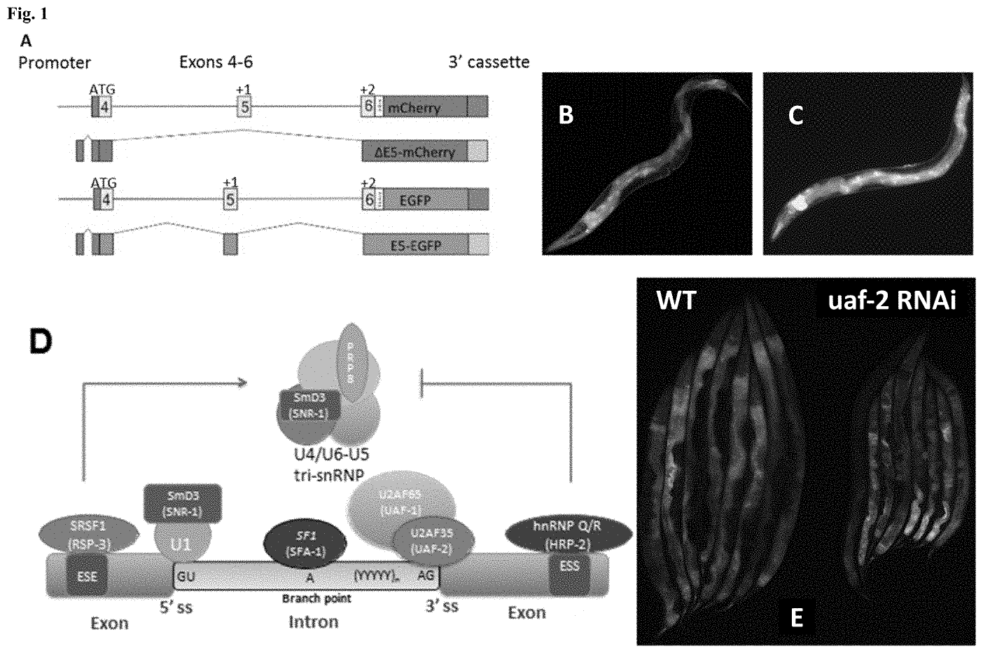

C. elegans is used to analyze tissue specific splicing patterns with age and with dietary restriction-mediated longevity. Mono- and bichromatic fluorescent minigene reporters have been successfully used to monitor correct splicing or skipping of alternative exons in cell lines, mice and rats (49, 70-75). The expression of each fluorescent protein represents a specific, quantifiable splicing event. Fluorescent splicing reporters make use of the advantage of the nematodes' translucent appearance, making splicing events easily visible under a microscope, in vivo as animal age, negating the need for amplifying the RNA or probing for protein interactions in vitro. In vivo fluorescence allows the visualization of splicing alterations that may otherwise not readily show detectable phenotypes (49, 50). Strain (KH2235) expressing a symmetric pair of ret-1 exon 5 reporter minigenes under the ubiquitous eft-3 promoter was used. The minigene is regulated by the UNC-75 RNA binding protein and was generated as described in Kuroyanagi et al. (49, 50). Differential frame shifts in the mini gene constructs result in premature stops in either mCherry or GFP expression when exon 5 is included (mCherry) or skipped (GFP). Therefore, expression of E5-mCherry indicates exon 5 has been skipped while expression of E5-EGFP indicates exon 5 has been included, as illustrated in the minigene scheme (FIG. 1). Young adult worms show a homogeneous splicing pattern, with exon 5 consistently excluded in body wall muscles but included in intestine, represented by uniform EGFP expression in intestinal cells (FIG. 1). Control animals expressing standard mCherry and GFP under the same eft-3 promoter show co-expression of both fluorophores and are therefore yellow. Perturbing spliceosome function altered the pattern of fluorophore expression in KH2235 was verified by knocking down the key splicing factor UAF-2 by RNAi.

### Example 3. Aging Induces Splicing Dysfunction and Mimics Spliceosome Perturbation

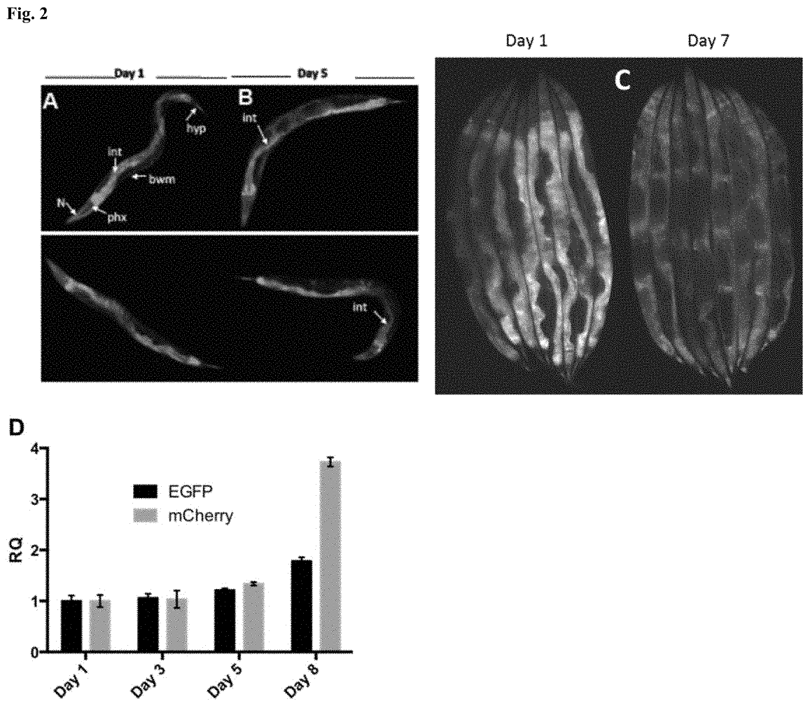

During development in C. elegans splicing is under tight regulation. As such the splicing reporter strain KH2235 undergoes stereotypical and homogeneous changes in alternative splicing across tissues through different developmental stages, and these changes are seen uniformly in all the population (49, 50). A series of experiments were performed to determine if aging induces generalized loss of splicing fidelity, and as such the KH2235 strain was aged and monitored for changes in fluorescence. Strikingly, by Day 5, when C. elegans are still considered “youthful” and look homogeneous under bright field light microscopy, heterogenous patterns of exon usage between animals were observed (FIG. 2). Whilst some animals maintained a youthful splicing pattern, others had miss-slicing events in intestinal cells that resembled what was observed with uaf-2 RNAi (spliceosome dysfunction FIG. 1E). By day 7, all animals had lost their youthful splicing patterns and cells within the same tissues showed differential exon skipping (FIG. 2, Panels C and D). This age-related loss of splicing fidelity was confirmed with a separate splicing reporter strain which expresses GFP containing intronic sequences that must be correctly spliced for GFP translation (76). Loss of GFP expression with age was observed, indicative of dysfunctional pre-mRNA processing. These data are the first demonstration that aging induces global splicing dysfunction and mimics loss of spliceosome fidelity.

### Example 4. Splicing Fidelity Correlates with Physiological Age and DR

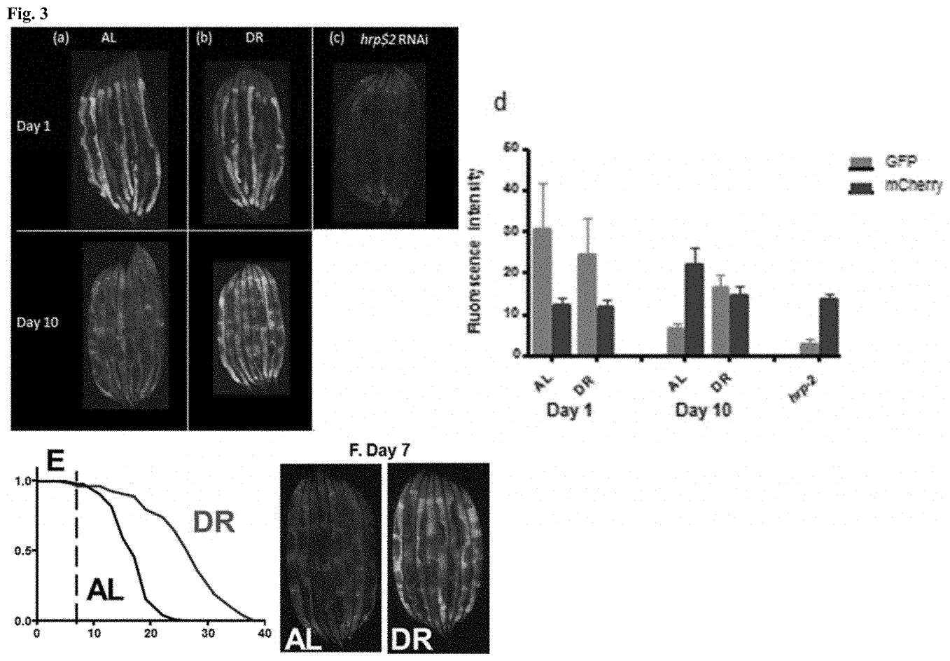

To investigate whether interventions that promote longevity maintain splicing fidelity, a series of experiments were performed to analyze splicing patterns in vivo using the above-described reporter strain with two different methods of DR, eat-2 mutation (77) and solid plate DR (78, 79). The evolution of a tissue-specific alternative splicing event between day 1 and 10 of adulthood in the KH2235 C. elegans strain (50) under plate ad libitum (AL) and DR growth conditions was investigated (FIG. 3). DR animals at day 10 of adulthood maintain a youthful splicing pattern compared to AL-fed worms at the same stage in life. Further, the aging phenotype at AL growth mimics a dysfunctional splicing pattern caused by hrp-2 RNA interference (a core spliceosome component). Similar results were seen for DR via eat-2 mutation. These results support the hypothesis that long-lived DR animals maintain homeostatic splicing for an extended time of life and that declining RNA homeostasis causes the aging phenotype and disrupted splicing patterns. Taken together these data demonstrate for the first time that global splicing fidelity decreases with age, correlates with physiological age and is maintained in long lived DR animals.

### Example 5. Splicing Factor Knockdown Suppresses DR-mediated Longevity

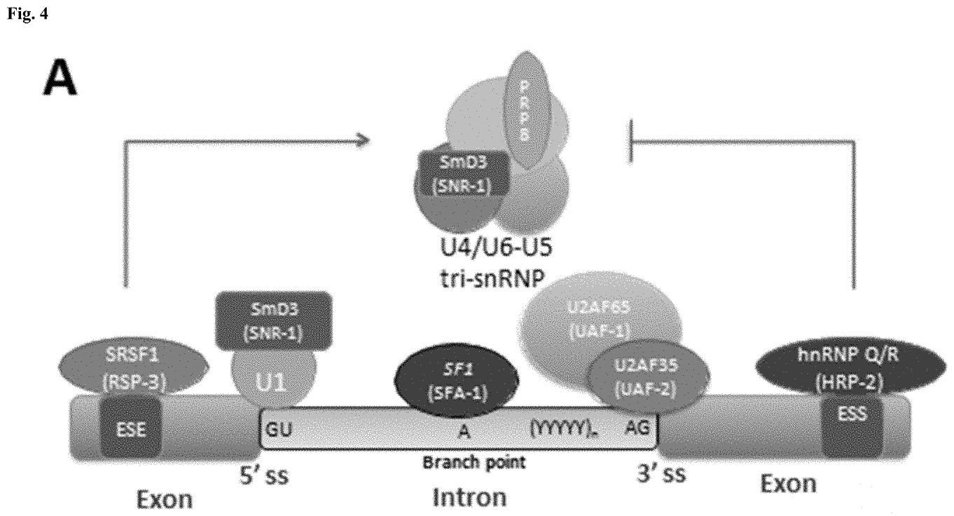

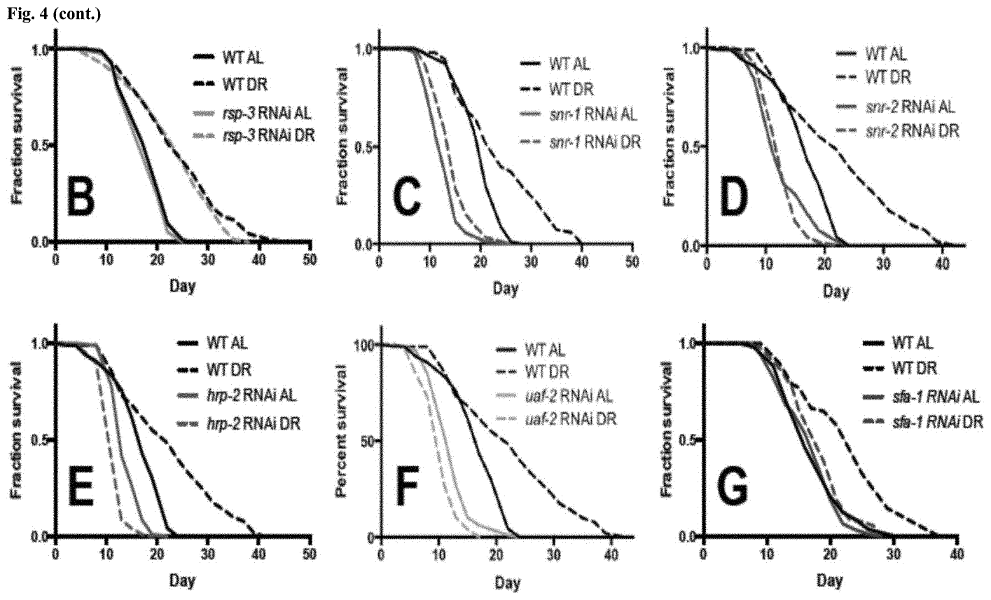

To determine if maintenance of splicing fidelity is required and causal for DR longevity a series of experiments were carried out using a targeted reverse genetic RNAi screen for components of the splicing machinery and DR longevity. Lifespan analysis was performed on DA1116 eat-2 mutant C. elegans strain, which is an established model of dietary restriction with extended lifespan. DA1116 (further solely denoted as DR condition) carries a mutation in eat-2 gene leading to reduced food uptake by limited pharyngeal pumping (77, 80). RNA interference of 11 different splicing factors and spliceosome components were tested for their effect on DR lifespan. FIG. 4 shows that knockdown of several splicing factors and spliceosome components specifically suppresses OR— induced longevity.

Multiple splicing factors were identified that had no effect on WT lifespan or DR longevity (Table 2). (Percentages of median DR lifespan extension on RNAis with significance level compared to N2 Bristol control strain on RNAi treatment.)

TABLE 2

| | | |
|---|---|---|
| RNAI | % Lifespan | |
| treatment | extension | p value |

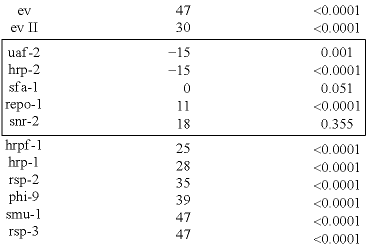

Strikingly, several splicing factors were completely required for DR-mediated lifespan extension (FIG. 4 Panels C-G and Table 2). RNAi of some of these factors shortened WT lifespan, as seen previously for loss of key homeostatic regulators such as the heat shock factor HSF-1, autophagy factors and proteosome components (16). Strikingly however, the screen also identified splicing factors such as SFA-1 whose inhibition did not affect WT lifespan, yet fully suppressed DR. These results therefore demonstrate that DR increases lifespan via a mechanism that requires specific components of the splicing machinery and are evidence linking RNA homeostasis to DR longevity.

### Example 6. Defining RNA Fidelity as a Biomarker of Physiological Age and Life Expectancy

The data above using two separate mini gene reporters in C. elegans indicates that splicing fidelity declines with age in multicellular organisms and correlates with physiological age and life expectancy in animals on AL and DR conditions. The disclosure also provides methods for assaying splicing with age using multiple mini-gene reporters with alternative splice recognition sites. Such methods may use RNA-Seq to corroborate that the effects observed using in vivo fluorophore reporters reflect what is happening at the RNA level. The methods will also determine if splicing individual variation in splicing fidelity can be used in as a predictor of life expectancy and biomarker of physiological age in young C. elegans.

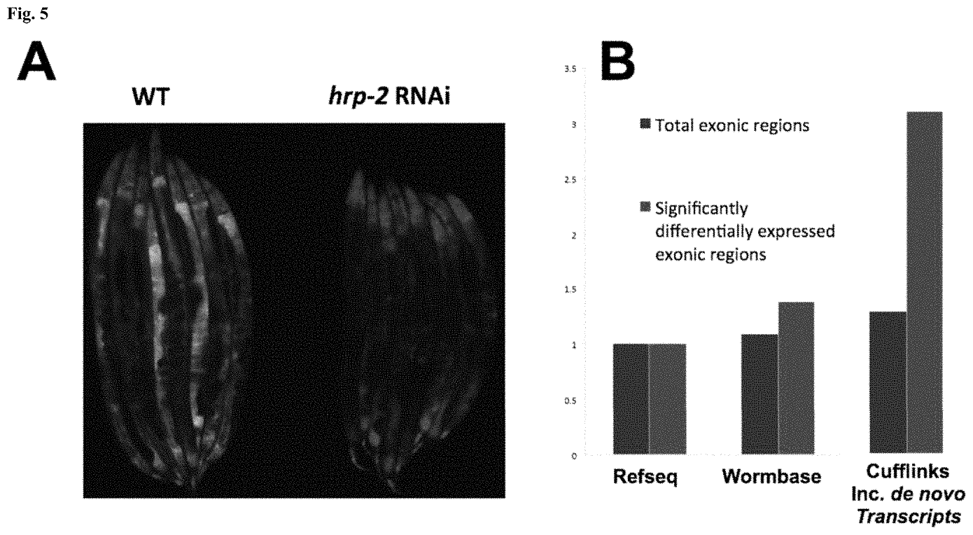

The above results with mini gene reporters are expanded using next generation RNA Seq analysis as a measure of splicing dysfunction with age and DR. As proof of principle that RNA Seq can distinguish spliceosome dysfunction as demonstrated using WT C. elegans with and without RNAi for a key splicing factor HRP-2, which suppresses DR longevity and affects the mini gene splicing reporter (FIGS. 4 and 5).

To give sufficient resolution, 100 b.p. bi-directional reads were used. In order to detect a general dysfunction in the splicing of transcripts, several databases of known transcripts of exons with increasing complexity were used, as well as Cufflinks (81, 82) to detect novel expressed exonic regions and transcripts from the aligned RNA-seq data. DEXSeq (83) was also used to identify significant changes and detected 5,225 exonic regions with differential expression between hrp-2 RNAi and control using the Cufflinks generated database, while it was found that 1,684 in the Refseq (84) database and 2,314 using Ensembi/Wormbase ver 75 (85). While RNAi for hrp-2 significantly increased expression of annotated exons of 29% in the Cufflinks generated database compared to Refseq (201,532 versus 155,971), there was a striking increase in the number of alternatively expressed exonic regions of 310% (FIG. 4), indicating that the novel exons identified by Cufflinks were more likely to be alternatively spliced. Because C. elegans is such a well-characterized organism with well-known transcripts, many of these novel exons are likely aberrantly included under dysfunctional splicing conditions. An enrichment in significant expression of exonic regions in the Cufflinks data base compared to other reference sets therefore suggests RNA processing and splicing dysfunction. This is similar to an analysis demonstrating exonization of Alu elements upon hnRNP C knock-down in an earlier study (86), and these aberrantly included exons were detectable by using Cufflinks and DEXSeq in combination. These data confirm the ability to use RNA Seq analysis to determine splicing dysfunction in C. elegans and support what was previously identified with RNAi induced spliceosome dysfunction and the mini gene reporters.

### Example 7. Research Design for Example 6

1.1 Determine if the Effects of Age on Splicing is General to all Splice Recognition Sites.

A series of experiments are performed to determine the global nature of the effects observed with mini gene reporters with age in C. elegans via generation of novel mini gene reporter lines for alternative spicing factors. Exon 5 of the KH2235 minigene carries one of the 20 most conserved 3′ splice sites found in C. elegans (AATTCAG) (87). However, it is under tight regulation by UNC-75, a RNA binding protein mostly associated with alternative splicing and neuron-specific splicing events. Therefore a series of experiments are performed to extend the repertoire of 3′ and 5′ splice sites, constitutive splicing and more globally regulated splicing reporters that are not necessarily tissue specific to uncouple splicing pattern effects from tissue changes with age. This is achieved by mutagenesis of splice sites in KH2235 minigene and by molecular cloning of new minigene reporters using MultiSite Gateway technology. In addition to the KH2235 fluorescent reporter strain used above, seven other fluorescent reporter worm strains will be used (49, 88-91) with targets of the CELF, STAR and RBFOX RNA binding proteins, which are subjected to the dietary restriction conditions and RNA interference treatments to thereby further confirm the observation that splicing homeostasis is required for healthy aging in long-lived animals. A monochromatic splicing reporter strain BL3466, which contains a transgene carrying an intron-containing gfp-expressing sequence is also studied. The reporter responds to constitutive splicing by splicing out the intron and expressing GFP. GFP expression decreases if the suppression of a splicing factor affects constitutive splicing (76). It is expected that the in vivo fluorescence imaging of the available and strains to be generated in the future to confirm extended splicing fidelity with age in DR irrespective of the splicing reporter.

1.2 Splicing Reporters and Prediction of Life Expectancy

Early deteriorations in splicing fidelity as visualized by changes in mini gene fluorescence are useful to predict life expectancy and act as a biomarker for aging. As can be seen by comparing control worms to the DR model in the above data (FIG. 3), animals with higher GFP expression are expected to live longer than worms with a deteriorated splicing pattern early in life. In order to confirm this hypothesis, live, synchronized fluorescent reporter worm strain populations are separated in early adulthood (Day 4) to distinct populations using the COPAS BIOSORT instrument. The COPAS BIOSORT is a high-throughput system to analyze and sort C. elegans based on optical and physical parameters with capability of dual fluorescence analysis. The BIOSORT instrument allows for an unbiased and automated separation of a large, mixed phenotype worm pool according to fluorescence levels and each worm population will be monitored until death to record lifespan data. Experiments will be performed to further confirm the worm sorting by testing animals on RNAi treatment with visible aberrant splicing patterns. It is expected that populations with youthful splicing patterns at day 4 to live longer than those showing splicing dysfunction. Splicing fidelity in young wild type animals is a predictor of how long they will live, and as such provides the first evidence linking RNA splicing dysfunction as a causal to aging.

1.3 Analyzing Splicing Fidelity in DR-Mediated Longevity

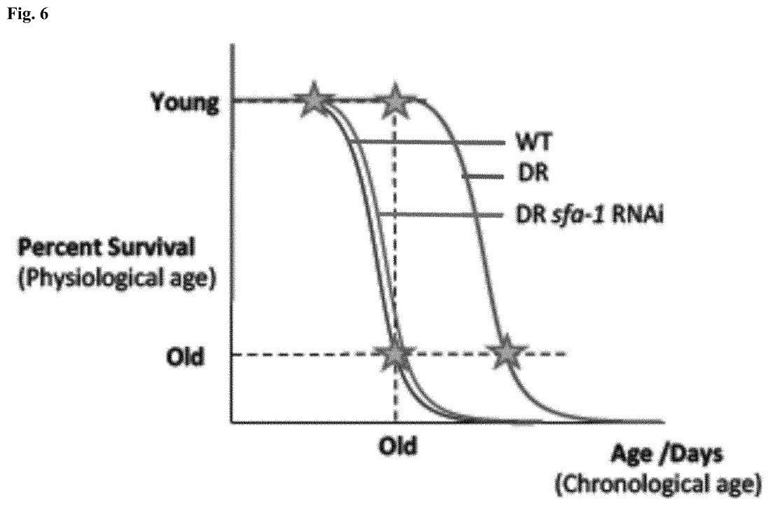

The above data show that RNA interference with spliceosome components and splicing factors abolishes longevity of DR worms. Furthermore, the above data confirmed that aberrant pre-mRNA splicing in C. elegans can be detected using RNA-Seq. A series of experiments are performed using RNA sequencing of the different conditions outlined in FIG. 6 to analyze effects of DR on a genome-wide scale in the transcriptome of long-lived animals. Using RNAi of sfa-1, which specifically suppresses DR longevity but has no effect on WT lifespan (FIG. 4) a series of experiments are performed to identify dysfunctional splicing patterns in chronologically age-matched wild-type, DR and DR+sfa-1 RNAi worms such as intron inclusion, alternative splice site selection, increased non sense mediated decay (NMD) etc. A temperature-sensitive sterile strain of the splicing reporter for RNA sequencing was generated to prevent contamination from embryonic RNAs and longevity will be induced by culturing the worms according to the solid-plate based DR assay (79). The sequencing will be done on at least 3 replicates for each worm population, using a paired-end protocol and aiming at high sequencing depth to generate a high-resolution framework. A combination of different gene models with increasing complexity are used, as well as the commonly used Cufflinks, DEXSeq and MISO programs (as shown in FIG. 5), and extend the analysis to other software tools if required. Candidate target genes are validated by quantitative RT-PCR.

### Example 8. Determining Causal Mechanisms Linking DR to RNA Homeostasis and Longevity

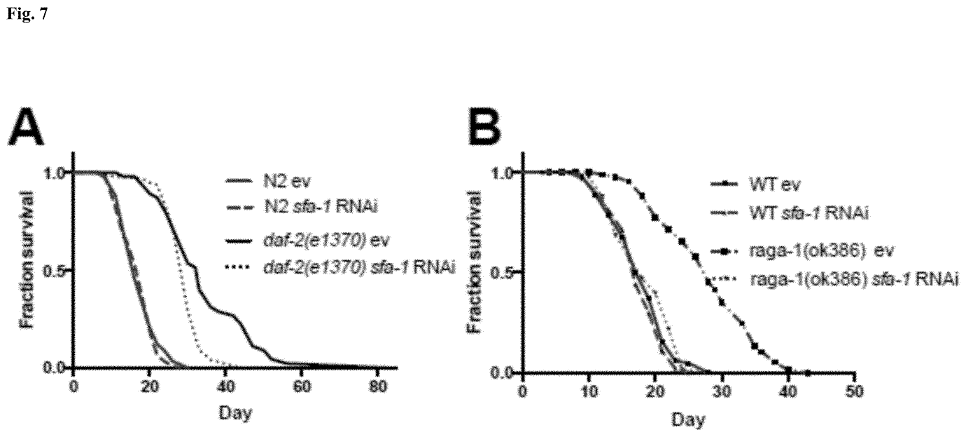

The data above show that perturbation of the splicing process decreases lifespan and that long-lived dietary restriction models show a youthful splicing pattern for a prolonged time in adulthood. Also identified are specific splicing factors that are required for DR and preliminary data show RNAi of DR specific splicing factors suppress TOR mediate longevity (FIG. 7). Here these observations are extend to identify the mechanistic link between RNA metabolism and DR longevity.

Inhibition of the splicing factor gene sfa-1 by RNAi specifically suppressed DR longevity while having no effect on WT lifespans (FIG. 4G). Experiments were performed to determine how specific this effect was to DR longevity or whether alternative SFA-1 is required for all genetic manipulations that promote longevity in C. elegans, as is the case for homeostatic regulators such as autophagy. Lifespan analysis was performed of WT to long lived mutants with impaired insulin/IGF-1 like signaling or mTOR signaling. Although sfa-1 RNAi shorten the maximum lifespan extension induced by daf-2 mutation it had no effect on median lifespan of daf-2 mutants (FIG. 7A). Strikingly however, sfa-1 RNAi completely suppressed lifespan extension seen in raga-1 mutants, which have suppressed mTOR signaling (FIG. 7B). These data highlight mTOR regulation of splicing as a potential mechanistic link between DR and RNA homeostasis.

### Example 9. Research Design for Example 8

2.1 Defining the Requirement of RNA Homeostasis for DR Longevity

Lifespan analysis with RNA interference of splicing factors and spliceosome components has shown that perturbed RNA homeostasis may lead to abrogation of lifespan extension seen by the two previously described models of dietary restriction (FIG. 4). To confirm the hypothesis of the functional requirement of RNA homeostasis for DR longevity, the preliminary screen is expanded to include additional components of the spliceosome. To do so, a copy of the C. elegans RNAi feeding library consisting of approximately 20,000 RNAi clones (95, 96) will be used, which library includes bacterial clones expressing dsRNA for most core components of the spliceosome. Also assessed is whether RNAi can be initiated from egg hatch or only after the animals have reached adulthood. In addition, a series of experiments is performed to further confirm the positive hits from eat-2 animals to a second paradigm of DR in C. elegans, solid plate DR (79). A list of core splicing factors for targeted RNAi screening was generated (Table 1). Combinatorial RNAi experiments are performed in order to take functional redundancy of members of the SR family and hnRNP proteins into consideration (97-99). For factors whose RNAi does not induce legality, deletion mutants will be generated via Crispr/cas9 technology (100). The above data identified several factors (Table 2) crucial to DR-mediated longevity.

In addition to defining the role of splicing factors for DR longevity, a series of experiments are performed to identify genetic mediators of DR longevity that mediate the effect of DR on splicing. The KH2235 reporter strain is crossed into loss of function genetic mutants known to impact DR longevity (2), including: raga-1, aak-2, sir-2.1, rsks-1, daf-2, daf-16, skn-1, hif-1 and pha-4. This is used to assess the effect of these mutants on splicing fidelity using our mini gene reporters on both AL and DR feeding regimes. This approach identifies molecular mechanisms by which DR protects against loss of RNA fidelity with age.

2.2 Defining the Role of mTOR as a Mechanistic Link Between DR and RNA Homeostasis

The above data indicates that the TOR pathway links DR to RNA homeostasis, since sfa-1 RNAi specifically blocks both DR and raga-1 mediated lifespan, whilst having no effect on WT animals (FIGS. 4 and 7). In addition, splicing efficiency has recently been shown to be increased via suppression of TOR signaling in yeast with rapamycin via reducing competition for limited spliceosome components (101). This suggests that reduced TOR and DR may increase lifespan in part through altering spliceosome dynamics. A series of experiments are performed to test this hypothesis using C. elegans and mammalian tissue culture. These experiments determine the requirement of SFA-1 for lifespan extension by alternative mechanisms of TOR suppression in C. elegans. These will include mutants for the TORC1 components TOR (LET-363) and Raptor (DAF-15) (102, 103), along with rapamycin treatment (104). In addition, the experiments assess the effect of reduced TOR signaling on the spliceosome components in mammalian tissue culture and C. elegans. q-RT-PCR and Western blot are used to analyze core spliceosome factors in WT mouse embryonic fibroblasts (MEFs) and MEFS lacking either TSC 1 or 2 after serum starvation, making TOR signaling either fully suppressed (WT) or active (TSC-′-) (105). In particular the experiments focus on the mammalian ortholog of SFA-1, the splicing factor 1 (SF1) (106).

2.3 Test the Role of SFA-1 in Lifespan Extension by Molecular Mediators of DR

In order to determine the universality of RNA splicing homeostasis in lifespan extension a series of experiments are performed to determine whether SFA-1 is required for other genetic manipulations that promote longevity in C. elegans. This is done both by using RNAi of SFA-1 but also via Crispr/Cas9 deletion of sfa-1. The experiments examine the requirement of SFA-1, along with other positive hits from our DR screen (FIG. 4) for known modulators of aging. These will include constitutively active AMPK, HSF-1 over expression, inhibition of mitochondrial function (suppression of isp-1, c/k-1, cc 1), and over expression of sir-2.1 (2).

### Example 10. Target RNA Homeostasis to Promote Healthy Aging

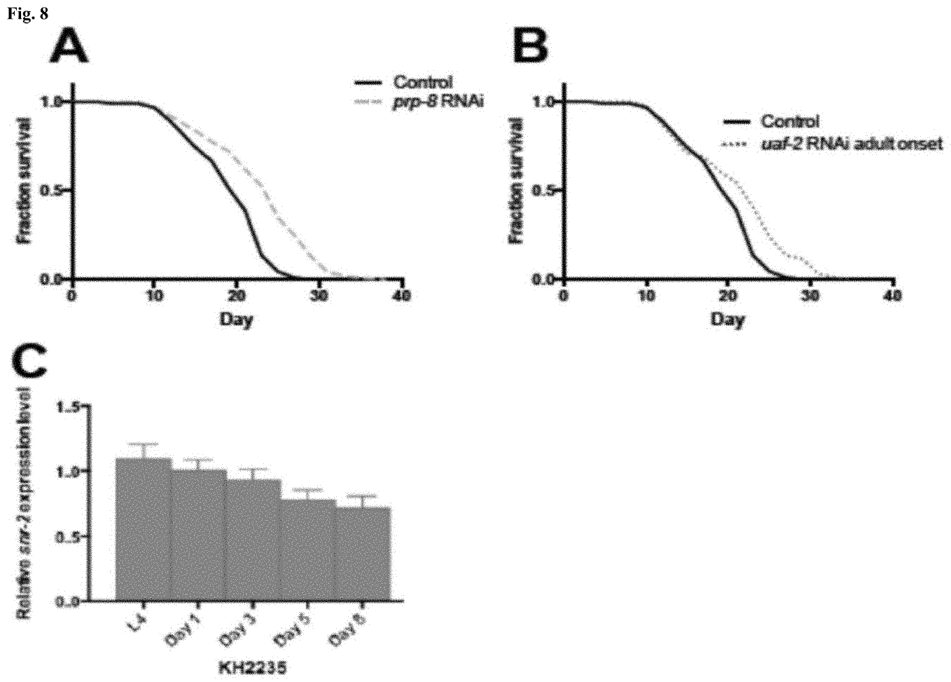

To fully harness RNA homeostasis as therapeutic for age-onset disorders, a series of experiments are performed to define the sufficiency of targeting splicing dynamics for promoting healthy aging. Data provided herein demonstrate the feasibility of this approach. For example, the data described herein indicate that inhibition of both prp-8 and uaf-2 can increase lifespan (FIG. 8). Reduced expression of splicing components with age in C. elegans. Thus it is believed that the dynamics of the spliceosome change with age and may be targeted directly to promote healthy aging.

3.1 Defining Spliceosome Dysfunction with Age

Preliminary data (FIG. 8) indicates that expression levels of spliceosome components decrease with age, which is believed to cause splicing errors and dysfunctional alternative splicing. Therefore, a series of experiments are performed to analyze the change of splicing factor expression with age in normal-lived animals and compare to expression levels in the long-lived DR models. In reference to several recent publications that have revealed changes in splicing factor expression with advancing age in specific tissues (21, 31, 32, 34), experiments are performed using quantitative RT-PCR using TaqMan expression assays in control and long-lived DR animals (87, 109) at various stages in animal lifespan. Higher splicing factor and spliceosome components expression levels in long-lived animals for an increased time of adulthood compared to control nematodes is observed. In order to analyze gene expression changes on translational level, experiments are performed to assess protein expression of candidate splicing factors crucial for longevity by Western blot. A significant decline in spliceosome component expression contributes to the explanation of dysfunctional RNA processing with age. To further characterize how the composition of the spliceosome alters with age, experiments are performed using immune-precipitation followed by mass spectrometry to identify components of the spliceosome at young and old ages, which has previously been employed successfully in the worm (110).

3.2 Investigating the Sufficiency of a Youthful Spliceosome for DR-Mediated Longevity

Maintenance of youthful spliceosome components expression levels is believed to be sufficient for longevity and healthy aging. Plasmids are constructed for overexpression of candidate splicing protein's eDNA linked to a 3xFLAG-tag and generate transgenic nematode strains for lifespan analysis. The 3xFLAG-tag allows subsequent protein analysis by western blot to assess expression levels and stability of the proteins. Overexpression experiments allow for analysis of the translation of gene expression effects to the protein level. It is expected that gain-of-function experiments of splicing proteins by overexpression show that RNA homeostasis can be targeted to promote longevity and healthy aging.

3.3 Determine the Capacity of DR to Protect Against Mis-Splicing in Tauopathies

It is believed that DR or genetic mimics of DR are useful alternative therapeutics for diseases associated with miss-splicing events, including diseases of aging such as tauopathies, a significant subset of which are caused by mutations that affect alternative spicing of tau pre-mRNA. C. elegans models that express WT (wild-type) and variants of human tau (variants which contain mutations seen in human patients which pre-dispose patients to tau mis-splicing and disease) are evaluated (112). As proof of principle that DR and inhibition of mTOR induce a generalized up regulation of splicing fidelity that might compensate for mis-splicing predisposition, a series of experiments are performed to test the pathogenic phenotypes of WT and mutant Tau in AL, DR and reduced mTOR (raga-1 mutation+rapamycin) conditions. It is expected that DR will specifically reduce the pathology of mis-spliced tau, and that this sub aim will provide the first example of DR/TOR as a potential therapeutic for age onset diseases of splicing.

### Example 11. Splicing Fidelity as a Biomarker for Life Expectancy

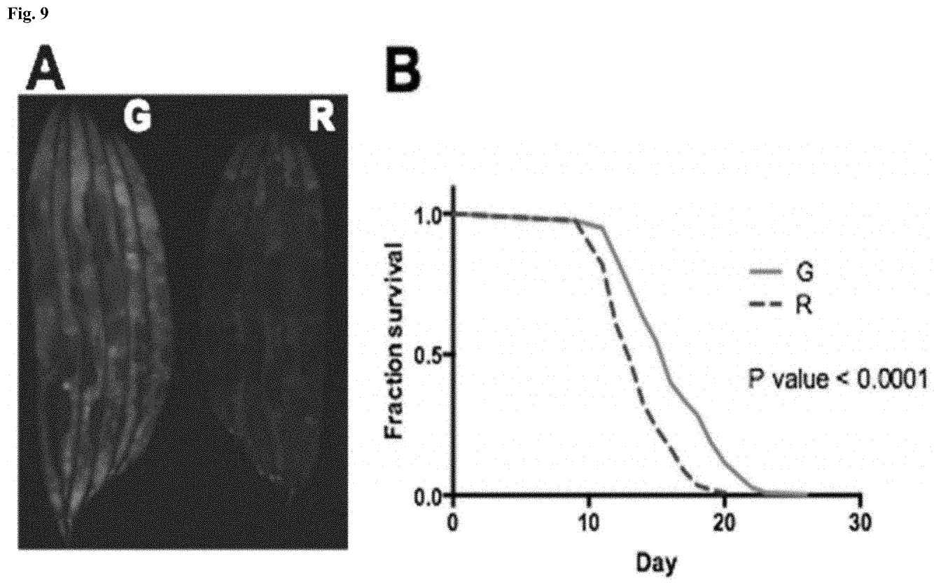

Fluorescence alterations visualized through a specific splicing event are believed to be predictive of life expectancy and act as a biomarker for aging. Accordingly, a synchronized population of day 6 old adult C. elegans was divided under the microscope to two groups of either high EGFP expression (exon inclusion) or dominant mCherry expression (exon skipping) (FIG. 9). Subsequently, the viability of worms in separate groups was determined blindly to calculate median lifespan and establish their lifespan curve. These data show that animals with a youthful splicing pattern display longer lifespan and therefore higher life expectancy. The experiment is repeated using RNA Seq to corroborate that the effects observed using in vivo fluorophore reporters reflect what is happening at the RNA level.

### Example 12. SFA-1 in Other Models of Longevity

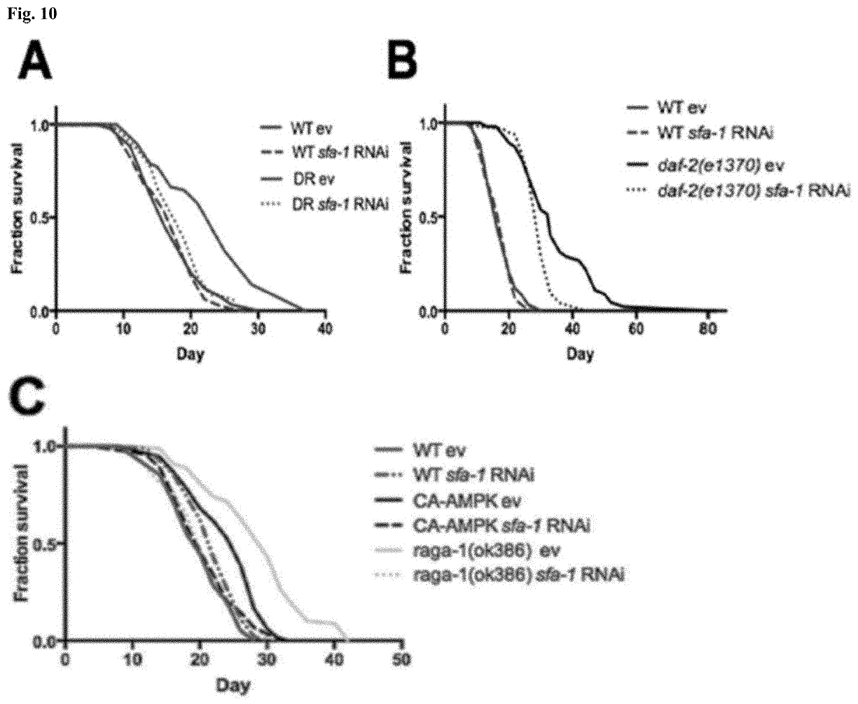

The inhibition of splicing factor SFA-1 did not affect wildtype lifespan, but fully suppressed DR (FIG. 10, Panel A). Lifespan experiments were performed to determine how specific this effect was to DR longevity or whether alternative SFA-1 is required for all genetic manipulations that promote longevity in C. elegans, as is the case for homeostatic regulators such as autophagy. Lifespan analysis of WT was compared to long lived mutants with impaired insulin/IGF-1 like signaling or mTOR signaling. Although sfa-1 RNAi shortens the maximum lifespan extension induced by daf-2 mutation it had no effect on median lifespan of daf-2 mutants (FIG. 10, Panel B). Strikingly however, sfa-1 RNAi completely suppressed lifespan extension seen in raga-1 mutants, which have suppressed mTOR signaling (FIG. 10, Panel C) and constitutively active AMPK strain. These data highlight mTOR regulation of splicing as a potential mechanistic link between DR and RNA homeostasis.

### Example 13. Defining Mechanistic Links Between DR and RNA Homeostasis

In addition to defining the role of splicing factors for DR longevity, a series of experiments were performed to identify genetic mediators of DR longevity that mediate the effect of DR on splicing. The KH2235 reporter strain was crossed into loss of function genetic mutants known to impact DR longevity (2), including: raga-1, aak-2, sir-2.1, rsks-1, daf-2 and daf-16. The effect of these mutants on splicing fidelity was assessed using the mini gene reporters on both AL and DR feeding regimes. The ability of DR to maintain a youthful splicing pattern was observed for daf-16 and aak-2 mutants, but was absent in the raga-1 mutant. This implies that the upstream TORC1 modulator RAGA-1 is required for the effect of DR on splicing. This approach contributed to identifying molecular mechanisms by which DR protects against loss of RNA fidelity with age.

### Example 14—Causal Mechanisms Linking DR to RNA

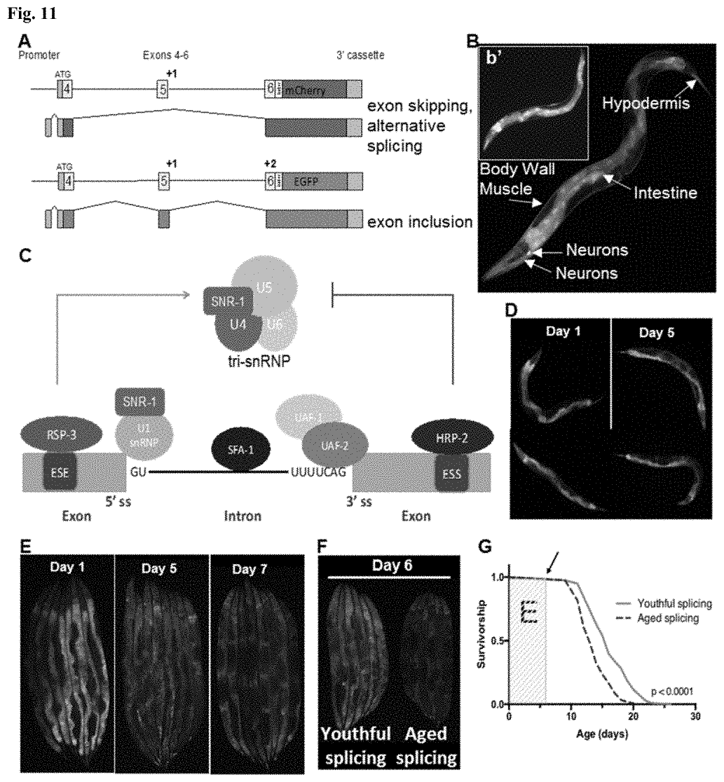

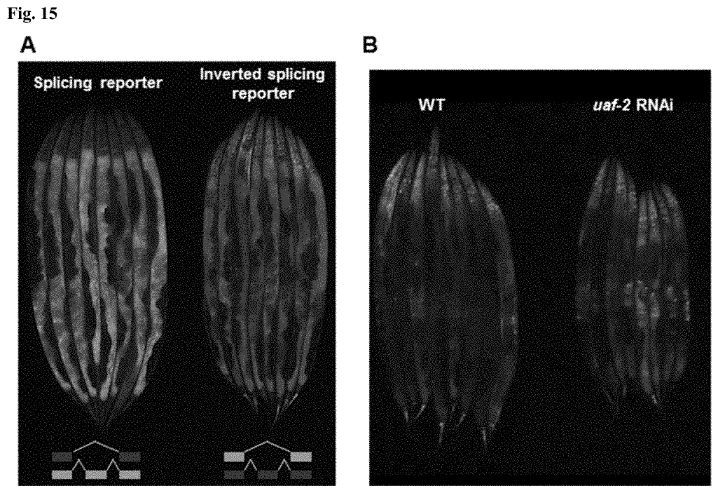

As discussed above, to examine causal links between aging and pre mRNA splicing in a multicellular system, a non-interventional in vivo fluorescent alternative splicing reporter in the nematode Caenorhabditis elegans was examined. This splicing reporter strain expresses a pair of ret-1 exon 5 reporter minigenes with differential frame shifts, driven by the ubiquitous eft-3 promoter. Expression of GFP indicates exon 5 has been included whereas expression of mCherry indicates exon 5 has been skipped (FIG. 11, Panel A). Alternative splicing of the reporter is regulated tissue-specifically, yet in young adult worms, each tissue type shows a homogeneous pattern of splicing across all cells (FIG. 11, Panel B). This tissue-specific splicing pattern is unrelated to differential stability of mCherry and EGFP, as an inverted reporter results in opposite mCherry and GFP expression (FIG. 15, Panel A). In addition, control animals expressing mCherry and GFP under the same eft-3 promoter uncoupled from the minigene reporter sequence show co-expression of both fluorophores and are therefore yellow (FIG. 11, Panel B insert).

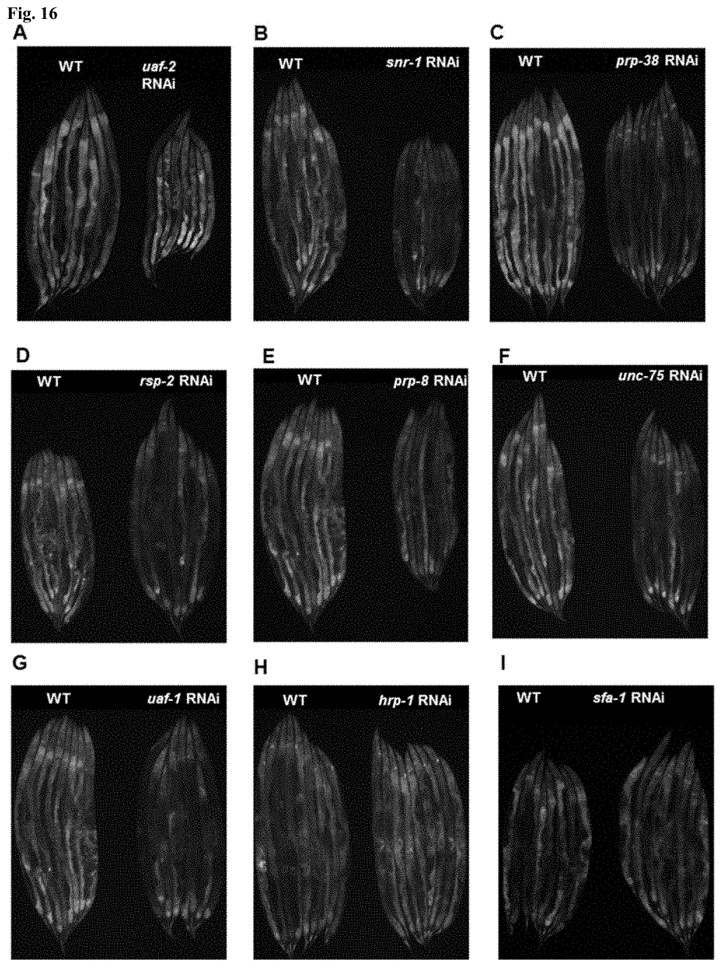

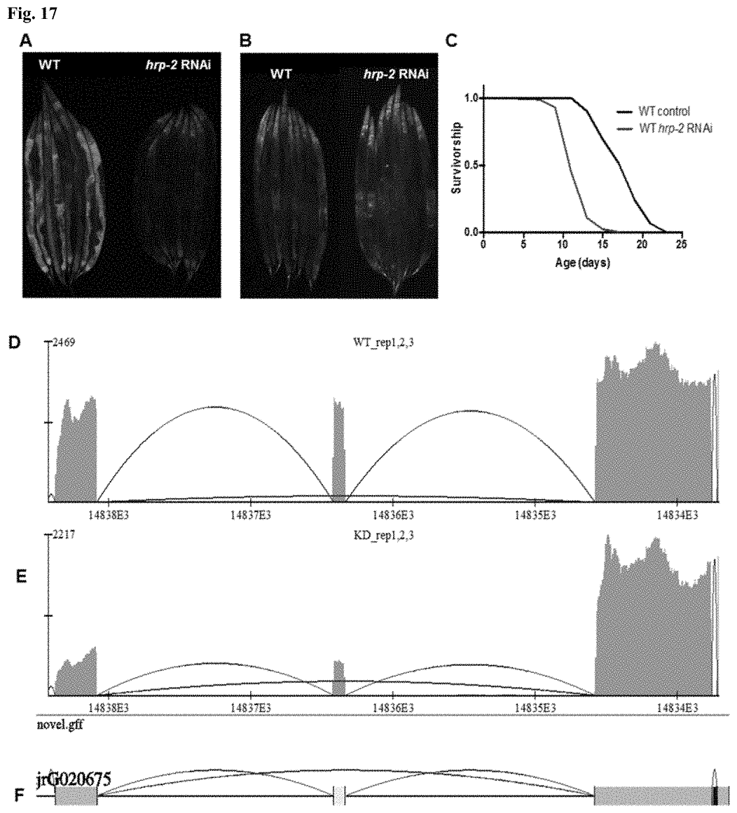

High conservation exists between the C. elegans and mammalian splicing machinery and regulation (FIG. 1 Panel C, Table 3). The utility of the reporter as a read out of spliceosome disruption was confirmed by inhibiting expression of splicing factors by RNA interference (RNAi). Reduced expression of multiple spliceosome components including hrp-2, a core, conserved component of the spliceosome in C. elegans and in mammals resulted in heterogeneous patterns of alternative splicing between individuals (FIG. 15, Panel B and FIG. 16). hrp-2 RNAi completely deregulates exon inclusion in the splicing profile of day 1 adult worms, and shortens wildtype animals lifespan (43% lifespan reduction log-rank p<0.0001, Extended Data FIG. 17) To further confirm that changes to the splicing reporter pattern by hrp-2 RNAi reflects widespread changes across the transcriptome, the genomewide consequences to splicing was analysed by RNA sequencing. As predicted, altered splicing of the endogenous ret-1 gene (FIG. 17) was found, as well as widespread splicing defects across multiple parameters including intron retention, exon skipping, and unannotated splice junctions. These RNA-Seq data therefore confirm that deregulation of the splicing reporter correlates with loss of splicing fidelity in vivo and can be used as a non-interventional surrogate readout of globalized defects in RNA processing and loss of splicing homogeneity within C. elegans populations.

TABLE 3
C. elegans splicing factors and their mammalian protein homologues

| C. elegans | Mammalian | | |
|---|---|---|---|
| gene name | protein homologue | Name | Function |
| uaf-2 | U2AF35 | U2 auxiliary factor small subunit | Core spliceosomal factor |
| uaf-1 | U2AF65 | U2 auxiliary factor large subunit | Core spliceosomal factor |
| sfa-1 | SF1/BBP | Splicing factor 1, branch point | Core spliceosomal factor |
| | | binding protein | |
| repo-1 | SF3A2 | Splicing factor 3a subunit 2 (66 kDa) | Core spliceosomal factor |
| snr-1 | SNRPD3 | Small nuclear ribonucleoprotein Sm D3 | Core spliceosomal factor |
| snr-2 | SNRPB | Small nuclear ribonucleoprotein Sm B | Core spliceosomal factor |
| rsp-2 | SRSF5, SRp40 | Serine/Arginine-rich splicing factor 5 | Extrinsic non spliceosomal |
| | | | RNA binding protein |
| rsp-3 | SRSF1, SF2/ASF | Serine/Arginine-rich splicing factor 1 | Extrinsic non spliceosomal |
| | | | RNA binding protein |
| hrp-1 | hnRNP A1 | Heterogeneous nuclear ribonucleoprotein | Extrinsic non spliceosomal |
| | | A1 | RNA binding protein |
| hrp-2 | hnRNP R | Heterogeneous nuclear ribonucleoprotein | Extrinsic non spliceosomal |
| | | R | RNA binding protein |
| hrpf-1 | hnRNP F/H | Heterogeneous nuclear ribonucleoprotein | Extrinsic non spliceosomal |
| | | F/H | RNA binding protein |
| prp-8 | PRPF8 | Pre-mRNA processing splicing factor 8 | Core spliceosomal factor |
| phi-9 | NHP2L1 | NHP2-like protein 1 | RNA binding protein |
| smu-1 | SMU1 | WD40 repeat-containing protein SMU1, | RNA binding protein |
| | | fSAP57 | |
| sym-2 | hnRNP F/H | Heterogeneous nuclear ribonucleoprotein | Extrinsic non spliceosomal |
| | | F/H | RNA binding protein |

### Example 15.—Ageing Induces Loss of Splicing Homeostasis

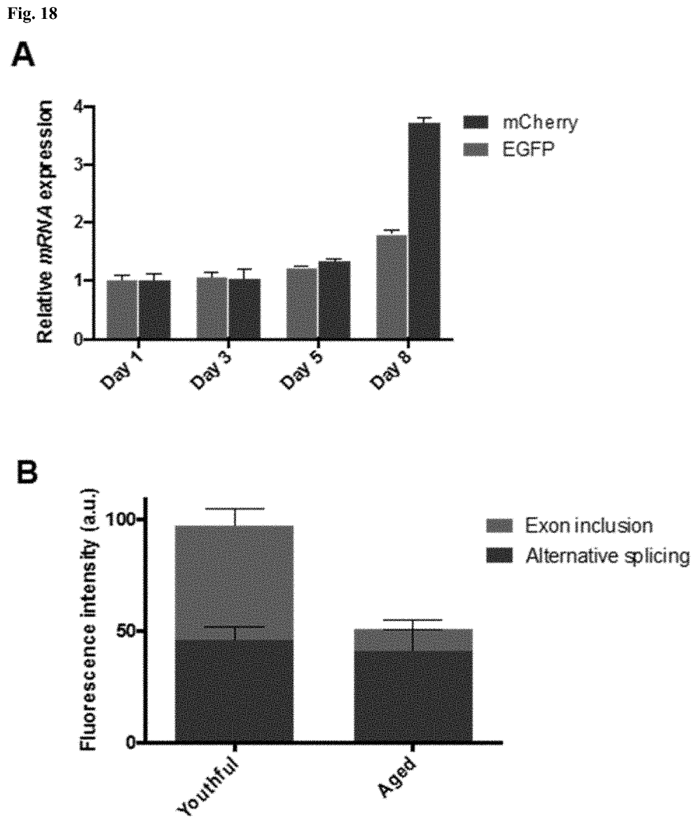

The splicing reporter was next used to monitor alternative splicing in a whole organism as it ages, to establish whether ageing induces generalized loss of splicing homeostasis. Splicing in C. elegans is under tight regulation, especially during development. As a result, the splicing reporter undergoes stereotypical and homogeneous changes in alternative splicing across tissues throughout larval development into early adulthood. These changes are consistent and seen uniformly across the population. However, by day 5, when C. elegans are still considered ‘youthful’ and are phenotypically homogeneous under bright field microscopy across the population, heterogeneous patterns of exon usage between animals was observed (FIG. 11, Panels D and E). This is especially evident within cells of the intestine which begin to variably express the alternate mCherry splice form, signifying that exon 5 is variably being skipped. By day 7, all animals had lost their youthful splicing patterns and cells within the same tissues showed differential exon skipping (FIG. 11, Panel E), despite reporter minigene being robustly expressed (FIG. 18, Panel A). Unlike young populations, day 7 old worm populations display high levels of splicing heterogeneity with varying reporter patterns, indicating variation in deregulated and aberrant RNA processing events between individuals. Ageing therefore leads to a deregulation in alternative splicing and this occurs at different rates between individuals.

Despite being isogenic, wild type (WT) C. elegans show remarkable heterogeneity in rates of ageing between individuals. To determine if inter-individual splicing fidelity might underlie inter-individual differences in ageing synchronized populations of day 6 old adult C. elegans were sorted into two groups based solely on their splicing patterns: those with homogeneous pattern of splicing more characteristic of young animals, and those with a heterogeneous ‘aged’ pattern. (FIG. 11, Panel F, and FIG. 18, Panel B), and assayed subsequent lifespan. Notably, the subsequent median lifespan of C. elegans with youthful splicing patterns at day 6 was significantly greater than animals with early onset deregulated alternative splicing (23% increased, p<0.0001, log-rank test, FIG. 11, Panel G). Therefore, splicing efficiency declines at different rates between individuals, correlates with physiological age, and can be used as a biomarker to predict life expectancy and intrinsic ageing in young animals.

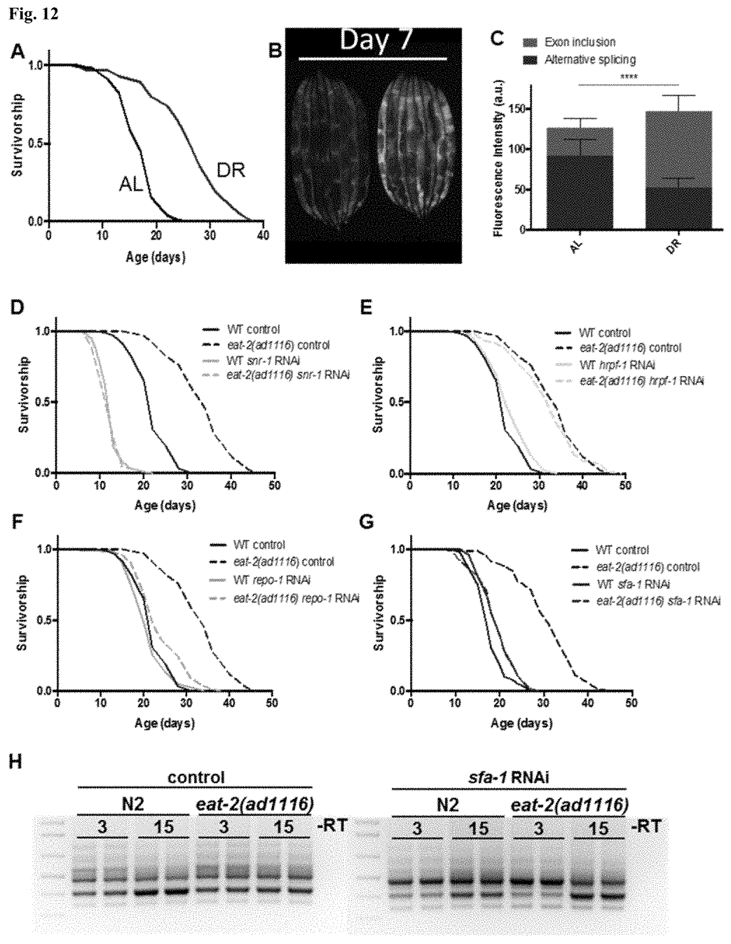

To determine whether interventions that slow ageing also maintain splicing homeostasis, splicing patterns in vivo in cohorts of C. elegans maintained on a dietary restriction regime that robustly extends lifespan (65% lifespan increase, log-rank p<0.0001, FIG. 12, Panel A) were analysed. Strikingly, while animals fed ad libitum (AL) show deregulated splicing with age, C. elegans on DR maintain a youthful and homogeneous splicing pattern (FIG. 12, Panel B and C). Taken together these data demonstrate that global splicing fidelity is a biomarker of life expectancy, decreases with chronological age and correlates with the youthful physiological age of long-lived DR animals.

### Example 16—Blocking the Effects of DR

If splicing fidelity is causal to ageing as opposed to a correlate of individual health, blocking the effects of DR on splicing should inhibit DR longevity. A targeted reverse genetic screen for spliceosome components that affect lifespan during WT or DR feeding was performed. WT and DR longevity of animals subjected to RNAi of 14 spliceosome components were assayed using the eat-2(ad1116) mutant as a genetic model for DR (Table 4). These factors represented different classes of splicing factors including core components of the spliceosome, RSP family enhancers and HRP family repressors (FIG. 11, Panel C). As expected given the critical role of splicing in organismal homeostasis, spliceosome components were identified whose inhibition by RNAi significantly reduced lifespan of both WT and DR animals, such as the core spliceosome factors SNR-1 (FIG. 12, Panel D), UAF-2 and SNR-2 (FIG. 18, Panels B and C). Despite blocking DR longevity, such detrimental effects on lifespan do not prove a causal role for splicing homeostasis in DR longevity, and are moreover reminiscent of the effects for loss of key homeostatic regulators such as the heat shock factor HSF-1, autophagy factors or proteasome components. Factors that had an effect on splicing homeostasis as assayed by the in vivo reporter were also identified, e.g. phi-9, hrpf-1 (FIG. 18, Panels D and E), rsp-2 (FIG. 15, Panel E) and hrp-1 (FIG. 15, Panel I), yet had no effect on longevity (hrpf-1 (FIG. 12, Panel E), rsp-2 (FIG. 18, Panel F, Table 4). This confirms that differential composition of the spliceosome can have separable effects on different physiological outcomes, and that remodelling splicing dynamics does not obligatorily shorten lifespan.

TABLE 4
eat-2(ad1116) RNAi screen. RNA interference
induced from egg hatch

| | | Median | p-value | | |
|---|---|---|---|---|---|
| | Median | lifespan | N2 RNAi vs. | % Lifespan | |
| | lifespan | eat- | eat- | extension | |
| | N2 | 2(ad1116) | 2(ad1116) | eat- | |
| Treatment | (days) | (days) | RNAi | 2(ad1116) | FUDR |
| ev* | 22 | 34 | <0.0001 | 55 | + |
| hrpf-1 | 22 | 34 | <0.0001 | 55 | + |
| repo-1 | 20 | 22 | <0.0001 | 10 | + |
| snr-1 | 13 | 13 | 0.5147 | 0 | + |
| sym-2 | 22 | 34 | <0.0001 | 55 | + |
| ev^{†} | 17 | 31 | <0.0001 | 82 | + |
| sfa-1 | 20 | 20 | 0.9783 | 0 | + |
| ev^{‡} | 17 | 25 | <0.0001 | 47 | |
| uaf-2 | 12 | 12 | 0.0299 | 0 | |
| hrp-1 | 18 | 23 | <0.0001 | 28 | |
| phi-9 | 18 | 25 | <0.0001 | 39 | |
| ev^{§} | 20 | 25 | <0.0001 | 25 | |
| rsp-3 | 17 | 25 | <0.0001 | 47 | |
| rsp-2 | 20 | 27 | <0.0001 | 35 | |
| smu-1 | 17 | 25 | <0.0001 | 47 | |
| ev^{∥} | 17 | 22 | <0.0001 | 29 | |
| hrp-2 | 13 | 11 | <0.0001 | −15 | |
| snr-2 | 11 | 13 | 0.3546 | 18 | |
| ev | 21 | 24 | <0.0001 | 14 | |
| uaf-1 | 11 | 9 | 0.6094 | −18 | |

(Note: ev, empty vector control, *survival curves in FIG. 12, Panel D, E and F, ^{†}FIG. 12, Panel G, ^{‡}FIG. 17, Panel E, ^{§}FIG. 17, Panel D, ^{∥}FIG. 17, Panel F)

### Example 17—Splicing Factors REPO-1 and SFA-1 Required for DR Longevity

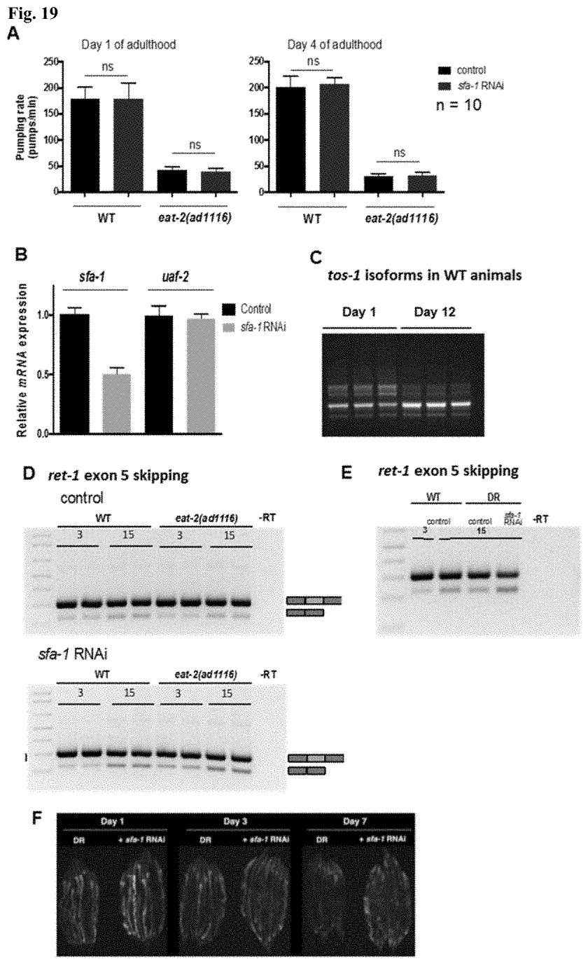

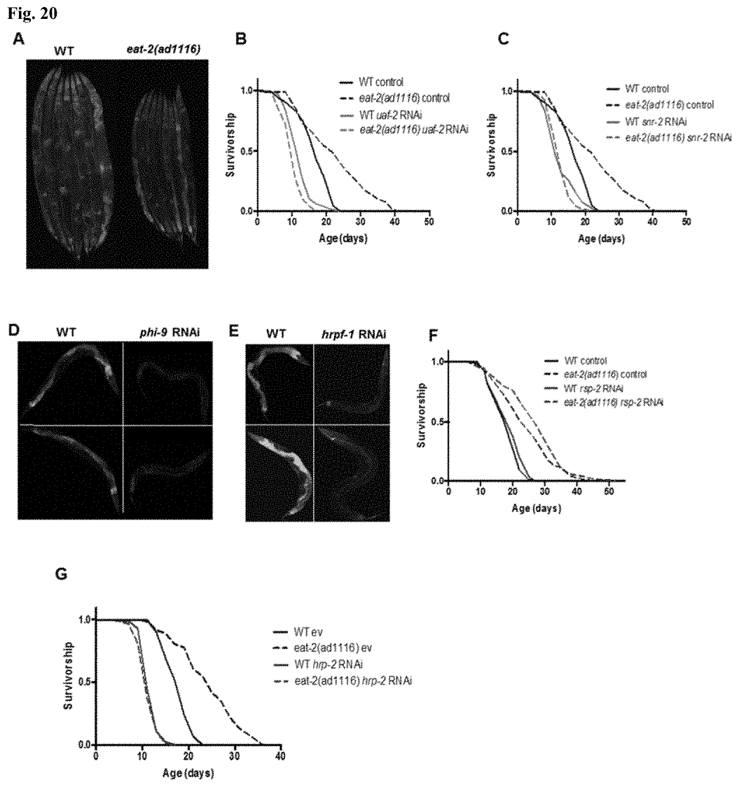

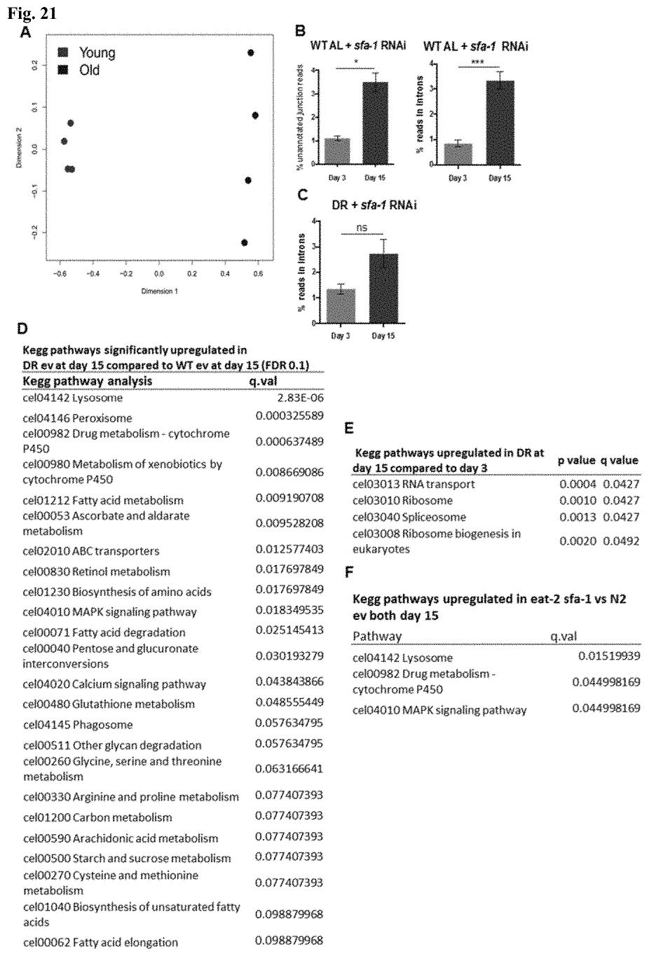

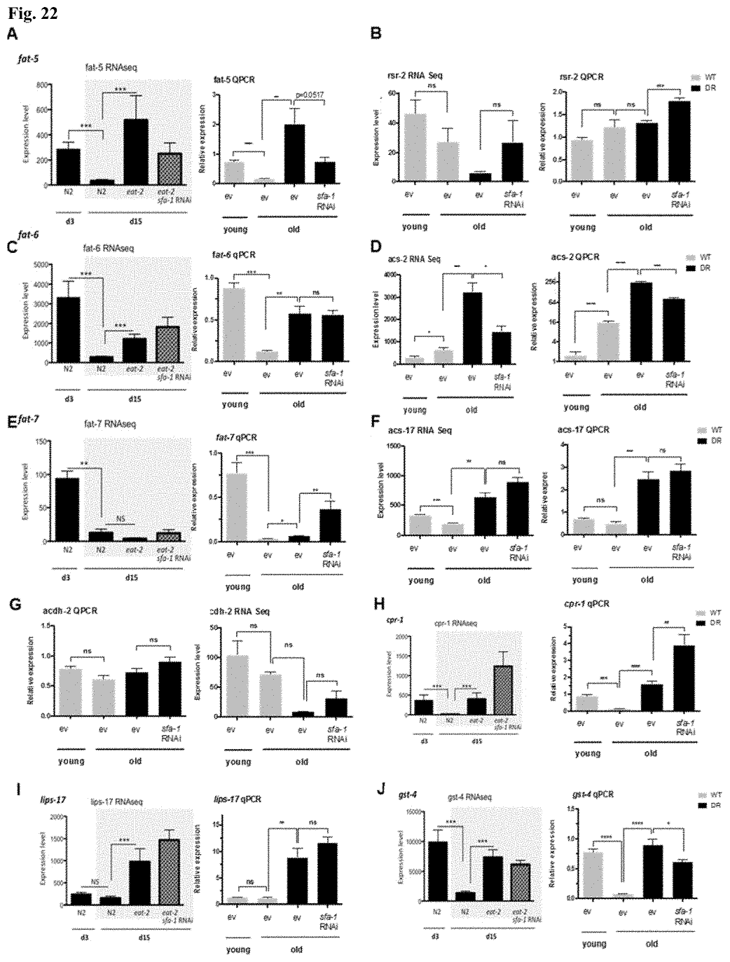

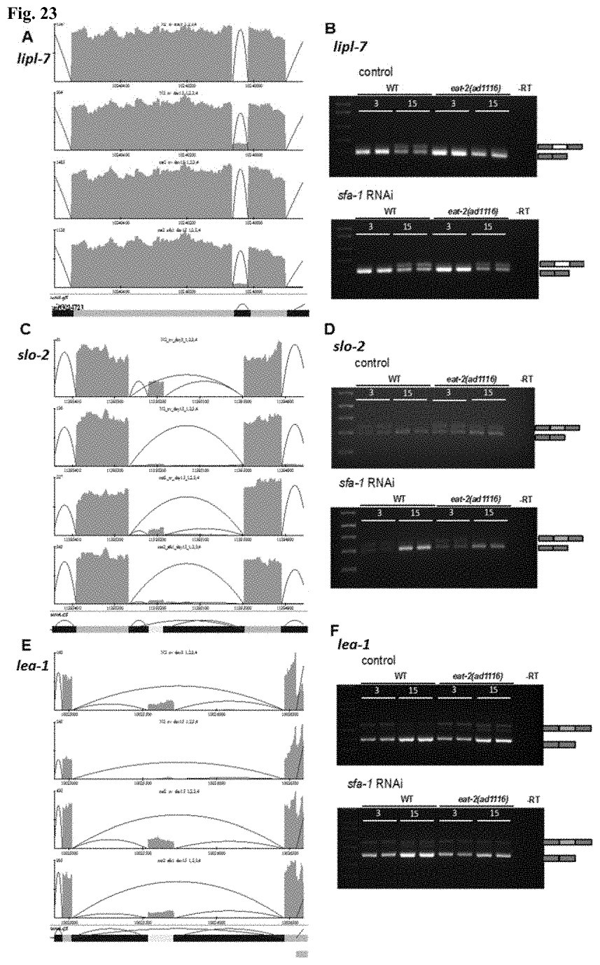

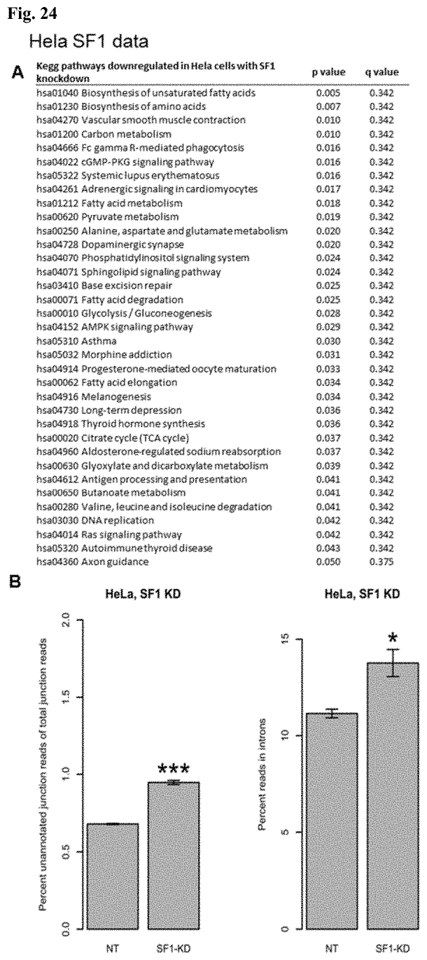

Importantly, and supporting a causal role for altered spliceosome dynamics in effects of DR, two splicing factors, REPO-1 and SFA-1 were identified that were specifically required for DR longevity. RNAi of repo-1 or sfa-1, had no adverse effects on WT lifespan (FIG. 12, Panels F and G). However, RNAi knockdown of repo-1 strongly reduces DR-mediated lifespan extension from 55% to 10% (p<0.0001, log-rank, FIG. 12, Panel F), while knockdown of sfa-1 completely abolishes DR-mediated longevity (p=0.9783, log-rank) (FIG. 12, Panel G). Both REPO-1 and SFA-1 interact with the UAF proteins and U2snRNP in 3′ splice site recognition during spliceosome assembly. Critically sfa-1 RNAi has no effect on feeding/pumping rates in C. elegans, or expression of uaf-2, which is expressed in an operon with sfa-1 (FIG. 19, Panel A and B). To assay SFA-1 function with age and DR, the splicing pattern of a known target of SFA-1, ‘target of splicing (tos-1) was analyzed. PCR analysis revealed an age-associated change in tos-1 splicing that is prevented by DR in an SFA-1 dependent manner (FIG. 12, Panel H, FIG. 19, Panel c). This suggests a loss of function of SFA-1 with age that is attenuated by DR. In addition, sfa-1 RNAi blocked the effect of DR on age-related changes to ret-1 splicing as assayed both by the in vivo reporter and by PCR of endogenous ret-1 exon 5 skipping isoforms (FIG. 19, Panel D). Together, these data demonstrate a causal role for the spliceosome and SFA-1/REPO-1 specifically in lifespan extension via DR. Moreover, the finding that only specific components mediate the effects of DR on ageing suggests that remodelling of spliceosome dynamics and composition, rather than efficiency alone, underlies DR longevity.

### Example 18—SFA-1 Promotes Longevity Under DR

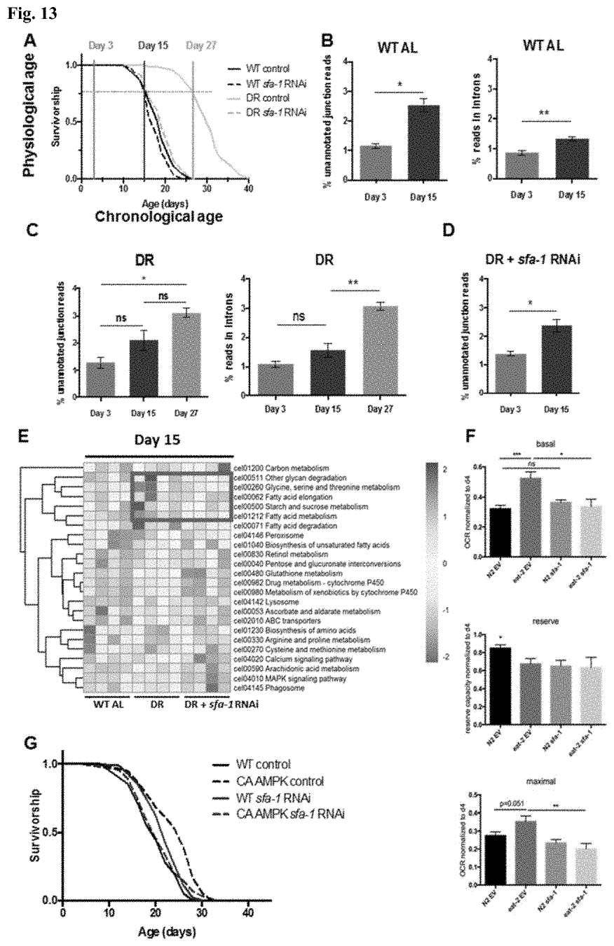

To determine the mechanism by which SFA-1 promotes longevity under DR, unbiased analyses were performed of the effect of age, DR and SFA-1 on both global RNA processing and gene expression. Long read 100 b.p bidirectional RNA Seq in young and old ad lib and DR (eat-2) worms+/−RNAi for sfa-1 were performed simultaneously with a lifespan assay. Samples were collected at days 3 when no animals had died, at day 15 (AL & DR sfa-1 RNAi 75% survival, DR 100% survival), and day 27 (DR 72% survival). This design allowed the matching of groups with the same chronological age (FIG. 13, Panel A, vertical lines) and physiological age (FIG. 13, Panel A, horizontal dashed line). The effect of age on multiple parameters of pre mRNA processing was assessed in ad libitum and DR fed C. elegans. Strikingly, ageing induced similar hallmarks of global spliceosome disruption seen previously in C. elegans with severe spliceosome dysfunction induced by hrp-2 RNAi (FIG. 17); Day 15 old ad libitum fed animals had a significant increase in both intron inclusion and unannotated splice junctions (FIG. 13, Panel B). Supporting the effects seen using the in vivo splicing reporter, DR protected against age-related deregulation of pre mRNA processing; chronologically age-matched Day 15 DR animals did not show significant increases in either intron inclusion or unannotated splice junctions when compared to young Day 3 animals (FIG. 13, Panel C). By Day 27 however, when the physiological age of DR animals matched that of Day 15 AL worms, both parameters of spliceosome dysfunction were significantly increased. This suggests splicing homeostasis is a biomarker of physiological age. Suggesting a causal role for SFA-1 on the effects of DR on global RNA processing, DR conferred no protection against the increase in unannotated RNA splice junctions by Day 15 in animals subjected to sfa-1 RNAi (FIG. 13, Panel D). Gene expression data and splicing events affected in ageing and with sfa-1 RNAi were validated by quantitative RT and semi-quantitative PCR respectively. Together, these data demonstrate that DR protects against a global dysfunction in RNA processing seen with age, and that this protection requires SFA-1.

### Example 19—Physiological Effects of DR Specifically Modulated by SFA-1

To define the physiological effects of DR specifically modulated by SFA-1, differential gene expression changes between AL and DR fed animals at Day 15, with and without sfa-1 RNAi (Table 5) were analyzed. Whether those KEGG pathways that were significantly altered by DR in Day 15 animals in an SFA-1 dependent manner was then determined. Strikingly, RNAi of sfa-1 did not block all DR related changed to gene expression, but instead specifically reversed up regulation of lipid/fatty acid metabolism genes induced by DR (FIG. 13, Panel E, boxed region). To determine if the effects of SFA-1 inhibition on metabolic processes is conserved, RNA Seq analyses on Hela cells was performed with and without siRNA of the mammalian SFA-1 ortholog, Splicing Factor 1 (SF1). Supporting a conserved role of SF1 in regulating metabolism, the most significantly enriched KEGG pathways in Hela cells with SF1 inhibition are metabolic processes. Confirming the effect of SFA-1 on fat metabolism, expression of fatty acid oxidation regulator acyl-CoA synthetase acs-2, is increased by DR and fasting in a sfa-1 dependent manner. To determine whether SFA-1 modulates the effects of DR on metabolism directly, oxygen consumption was measured in live old and young AL and DR C. elegans with and without sfa-1 RNAi. Maximal oxygen consumption was compared at day 4 and 15 to in order to determine changes in respiratory capacity with age. AL animals show decreased maximal respiratory capacity with age, and this decline is attenuated by DR in an SFA-1 dependent manner. These data suggest DR prolongs metabolic homeostasis and mitochondrial function during aging via SFA-1. To test this hypothesis, the role of SFA-1 in lifespan extension was tested via AMP-activated protein kinase (AMPK), a conserved regulator of metabolic homeostasis that is pro-longevity, activated by low energy and required for some DR longevity regimes in C. elegans. Suggesting a causal role for metabolic regulation in the effects of SFA-1 on lifespan, sfa-1 RNAi suppresses lifespan extension via constitutive AAK-2 activation (AMPK alpha 2 catalytic subunit, p=0.4844, log-rank, FIG. 13, Panel G). Together these data define SFA-1 as a novel modulator not only of DR and AMPK longevity but also of the effects of DR on metabolic flexibility and homeostasis.

TABLE 5
Kegg pathways significantly upregulated in DR ev
at day 15 compared to WT ev at day 15 (FDR 0.1)

| Kegg pathway analysis | q. val |
|---|---|
| cel04142 Lysosome | 2.83E−06 |
| cel04146 Peroxisome | 0.000325589 |
| cel00982 Drug metabolism - cytochrome P450 | 0.000637489 |
| cel00980 Metabolism of xenobiotics by cytochrome P450 | 0.008669086 |
| cel01212 Fatty acid metabolism | 0.009190708 |
| cel00053 Ascorbate and aldarate metabolism | 0.009528208 |
| cel02010 ABC transporters | 0.012577403 |
| cel00830 Retinol metabolism | 0.017697849 |
| cel01230 Biosynthesis of amino acids | 0.017697849 |
| cel04010 MAPK signaling pathway | 0.018349535 |
| cel00071 Fatty acid degradation | 0.025145413 |
| cel00040 Pentose and glucuronate interconversions | 0.030193279 |
| cel04020 Calcium signaling pathway | 0.043843866 |
| cel00480 Glutathione metabolism | 0.048555449 |
| cel04145 Phagosome | 0.057634795 |
| cel00511 Other glycan degradation | 0.057634795 |
| cel00260 Glycine, serine and threonine metabolism | 0.063166641 |
| cel00330 Arginine and proline metabolism | 0.077407393 |
| cel01200 Carbon metabolism | 0.077407393 |
| cel00590 Arachidonic acid metabolism | 0.077407393 |
| cel00500 Starch and sucrose metabolism | 0.077407393 |
| cel00270 Cysteine and methionine metabolism | 0.077407393 |
| cel01040 Biosynthesis of unsaturated fatty acids | 0.098879968 |
| cel00062 Fatty acid elongation | 0.098879968 |

### Example 20—Molecular Mechanism Linking DR to RNA Splicing

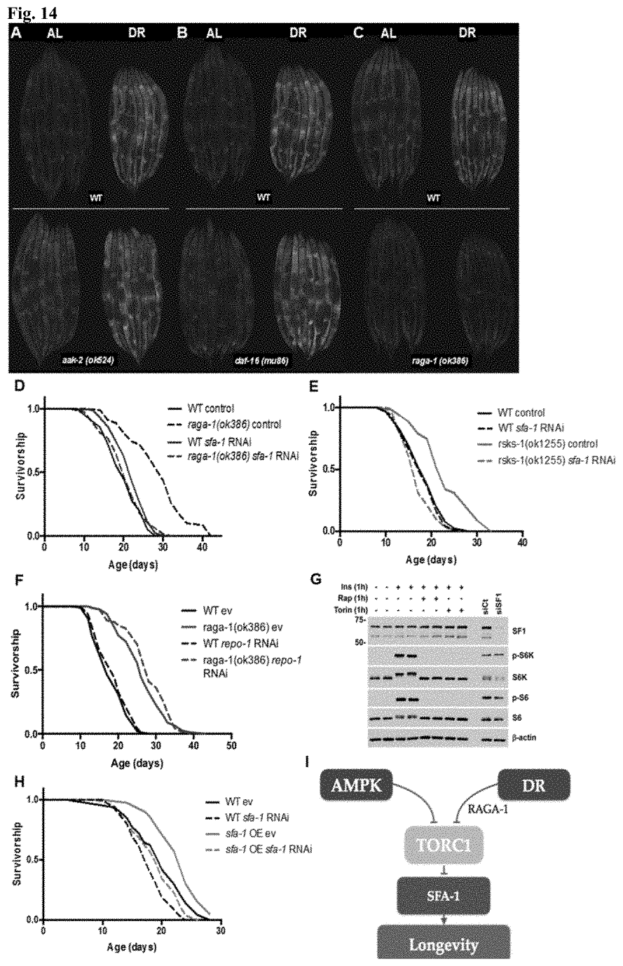

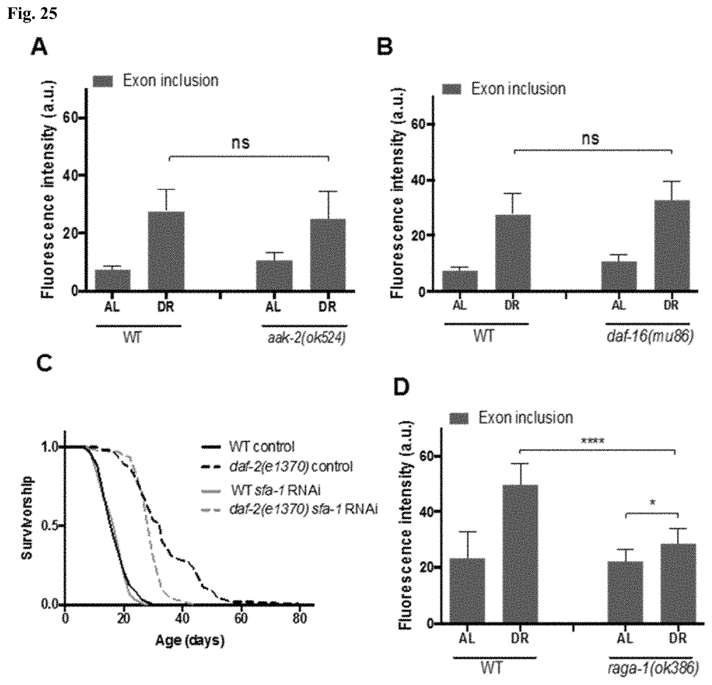

To investigate whether AMPK is the molecular mechanism linking DR to RNA splicing, the requirement of AMPK for the effects of DR on splicing was tested. Surprisingly, despite the role of SFA-1 in AMPK lifespan, DR maintains a youthful splicing pattern in day 8 old C. elegans lacking AAK-2, similar to that seen in DR WT worms (aak-2(ok524), (FIG. 14, Panel A, and FIG. 25, Panel A), as assayed by the in vivo mini gene reporter. Therefore although SFA-1 is required for AMPK longevity, the effects of DR on splicing must act in parallel or downstream of AMPK. Two direct downstream targets of AMPK with known roles in DR longevity are the transcription factor FOXO/DAF-16 and the target of rapamycin complex 1 (TORC1), a master regulator of cell growth and metabolism that is suppressed by AMPK and integrates nutrient, stress and energy status. As seen in WT animals, DR maintained a youthful splicing pattern in Day 8 C. elegans lacking FOXO/DAF-16 (daf-16(mu86), (FIG. 14, Panel B and FIG. 25, Panel B). In addition, sfa-1 RNAi did not abolish increased longevity of daf-2(e1370) mutants that have reduced insulin/insulin-like growth factor signalling (IIS) and require DAF-16/FOXO to modulate aging (FIG. 25, Panel C). These data suggest rIIS/FOXO signalling is not the mechanism by which DR modulates RNA splicing.

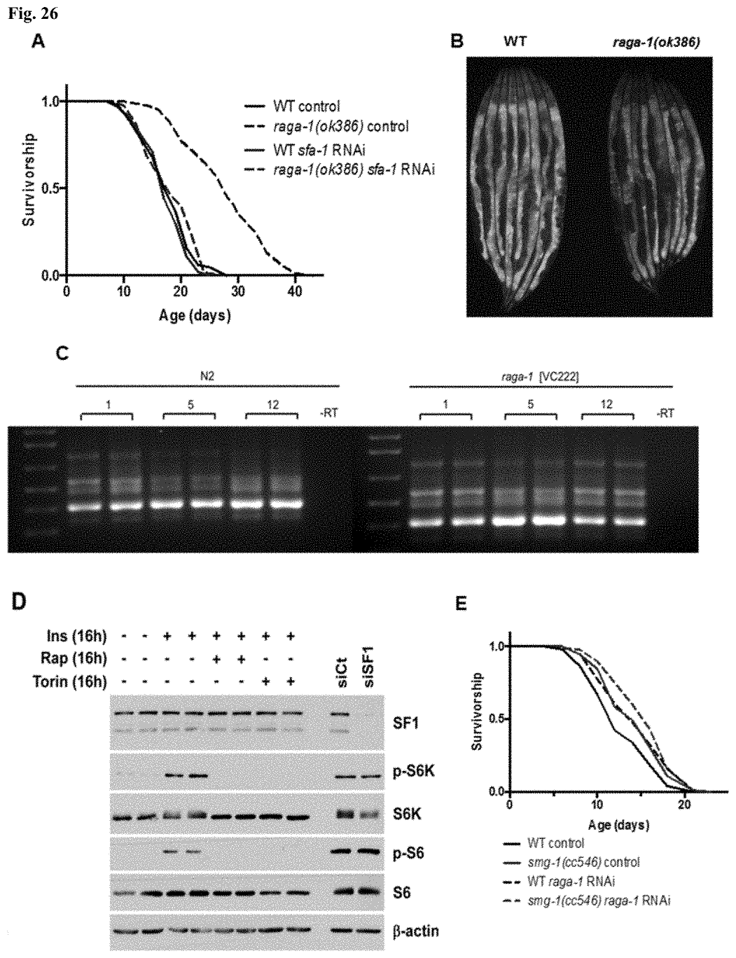

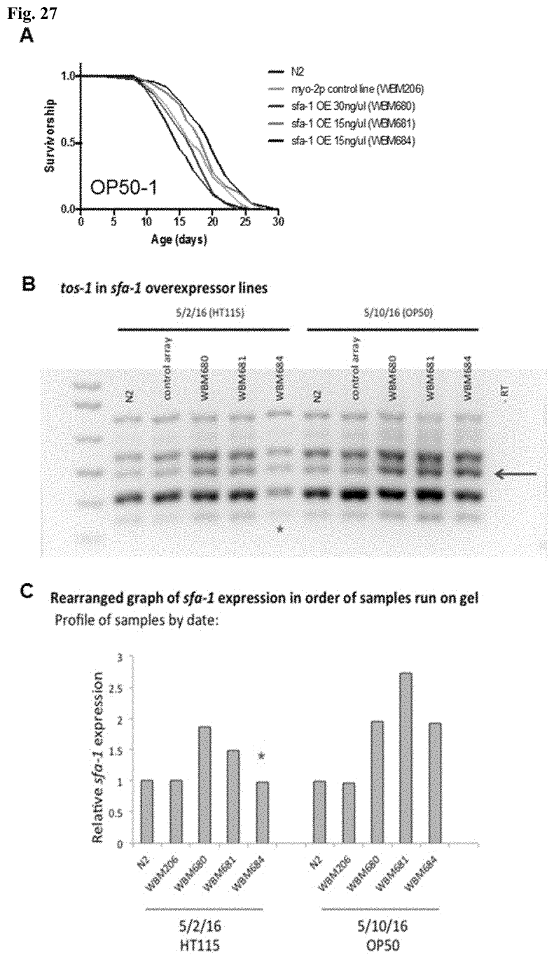

The role of TORC1 as a candidate node integrating the effects of AMPK and DR on both ageing and splicing homeostasis was examined. The TOR complex 1 activator Rag GTPase RAGA-1 acts as an upstream activator of TORC1, linking amino acid levels to TORC1 activity. Notably, suppression of mTOR signalling in raga-1(ok386) mutants completely abolishes the effects of DR on splicing with age (FIG. 14, Panel C, FIG. 25, Panel D), without affecting splicing in early adulthood (FIG. 25, Panel E). Direct suppression of TORC1 increases lifespan, and TORC1 is implicated as a mediator of the beneficial effects of DR. However, the mechanism by which TORC1 links DR to lifespan extension are unclear. Previous reports have linked TORC1 to regulation of splicing, with multiple splicing factors and splicing related kinases being identified in the mTOR-defined phosphoproteome and many harboring a conserved TOR signalling motif. Whether there was a direct role of RNA splicing in lifespan extension via suppression of the TORC1 pathway was therefore examined. Raga-1(ok386) mutants, which lack functional RAGA-1, show robust extension of median lifespan of 50% (FIG. 14, Panel D, Extended Data FIG. 26, Panel A). Strikingly, sfa-1 knockdown by RNAi fully suppresses the long lifespan of raga-1(ok386) (p=0.2766, log-rank, FIG. 14, Panel D, FIG. 26, Panel A). In addition, raga-1(ok386) mutants maintain youthful tos-1 splicing with age indicative of maintenance of SFA-1 activity. Whether SFA-1 is required for lifespan extension by loss of the TORC1 target S6 Kinase/RSKS-1 was tested. Null mutation to rsks-1 significantly increases lifespan in C. elegans. However, S6 Kinase/RSKS-1 lifespan is completely suppressed by sfa-1 RNAi (FIG. 14, Panel E), suggesting the role of SFA-1 extends beyond RAGA-1 as a general regulator of TORC1 mediated longevity. Interestingly, the effects on TORC1 longevity are specifically mediated by SFA-1, as REPO-1, which is required for DR longevity is dispensable for lifespan extension in raga-1 mutants (FIG. 14, Panel F). These suggest that differential composition of the spliceosome and specific splicing factors might mediate alternate longevity pathways, which warrants further study.

Together, these data suggest TORC1 is the central node mediating the effects of DR on splicing homeostasis, and highlight an emerging role for splicing efficiency and SFA-1 downstream of TORC1. Dynamic phosphorylation of splicing factors is essential to splicing regulation and spliceosomal activity. However, whether SFA-1 is a direct substrate of TORC1 in C. elegans or acts further downstream is unclear. Although an mTOR consensus motif was identified in the mammalian orthologue of SFA-1, ‘splicing factor 1 (SF1), inhibition of TORC1 via rapamycin or torin has no effect on total SF1 levels in mouse embryonic fibroblasts, nor did were SF1 gel shifts indicative of altered phosphorylation seen, as observed for the canonical TORC1 target S6 Kinase (FIG. 14, Panel G). Despite global increases in RNA splicing dysfunction with age that are reversed by DR (FIG. 13), the data suggest a more targeted role of SFA-1 then RNA splicing homeostasis. In support of this, inhibition of the non-sense mediated decay pathway, a key RNA homeostatic mechanism does not block lifespan extension by DR or raga-1 RNAi in C. elegans. Therefore, the data suggest the SFA-1 acts as a specific modulator of the effects of DR/TORC1 on metabolic homeostasis and flexibility. As such, regulation of splicing by TORC1 may be a key effector of its role in ageing and metabolism in addition to its effects on additional cellular processes such as protein translation and degradation.

Whether SFA-1 modulation of RNA processing is one of multiple cellular processes required in cohort for DR and TORC1 longevity, or alternatively whether activating SFA-1 alone might be sufficient for lifespan extension was determined. Independent transgenic lines that over expressed sfa-1 by 1.5-2 fold that of endogenous levels were generated. To confirm the sfa-1 transgene was functional, tos-1 splicing in the transgenic lines compared to WT was assayed. sfa-1 over expression correlated with a change of tos-1 isoform ratios and mirrored the effect of dietary restriction (FIG. 12). Notably, modest overexpression of sfa-1 is sufficient to increase lifespan by X % (FIG. 14), suggesting splicing factor 1 might be targeted to promote healthy ageing without the need to dietary restriction.

### REFERENCES

1. Fontana L, Partridge L, Longo V D. Extending healthy life span—from yeast to humans. Science. 2010; 328(5976):321-6.
2. Mair W, Dillin A. Aging and survival: the genetics of life span extension by dietary restriction. Annual review of biochemistry. 2008; 77:727-54.
3. Dixon A L, Liang L, Moffatt M F, Chen W, HeathS, Wong K C, Taylor J, Burnett E, Gut I, Farrall M, Lathrop G M, Abecasis G R, Cookson W O. A genome-wide association study of global gene expression. Nature genetics. 2007; 39(10):1202-7.
4. Morimoto R1, Cuervo A M. Proteostasis and the aging proteome in health and disease. The journals of gerontology Series A, Biological sciences and medical sciences. 2014; 69 Suppl1:S33-8.
5. Kenyon C J. The genetics of ageing. Nature. 2010; 464(7288):504-12.
6. Koga H, Kaushik S, Cuervo A M. Protein homeostasis and aging: The importance of exquisite quality control. Ageing research reviews. 2011; 10(2):205-15.
7. Ashburner M, Ball C A, Blake J A, Botstein D, Butler H, Cherry J M, Davis A P, Dolinski K, Dwight S S, Eppig J T, Harris M A, Hill D P, Issei-Tarver L, Kasarskis A, Lewis S, Matese J C, Richardson J E, Ringwald M, Rubin G M, Sherlock G. Gene ontology: tool for the unification of biology. The Gene Ontology Consortium. Nature genetics. 2000; 25(1):25-9.
8. Subramanian A, Tamayo P, Mootha V K, Mukherjee S, Ebert B L, Gillette M A, Paulovich A, Pomeroy S L, Golub T R, Lander E S, Mesirov J P. Gene set enrichment analysis: a knowledge-based approach for interpreting genome-wide expression profiles. Proceedings of the National Academy of Sciences of the United States of America. 2005; 102(43):15545-50.
9. Christensen K, Doblhammer G, Rau R, Vaupel J W. Ageing populations: the challenges ahead. Lancet. 2009; 374(9696):1196-208.
10. Gillum L A, Gouveia C, Dorsey E R, Pletcher M, Mathers C D, McCulloch C E, Johnston S C. NIH disease funding levels and burden of disease. PloS one. 2011; 6(2):e16837.
11. Freid V M, Bernstein A B, Bush M A. Multiple chronic conditions among adults aged 45 and over: trends over the past 10 years. NCHS data brief. 2012(100):1-8.
12. Goldman D P, Cutler D, Rowe J W, Michaud P C, Sullivan J, Peneva D, Olshansky S J. Substantial health and economic returns from delayed aging may warrant a new focus for medical research. Health affairs. 2013; 32(10):1698-705.
13. Kennedy B K, Berger S L, Brunet A, Campisi J, Cuervo A M, Epel E S, Franceschi C, Lithgow G J, Morimoto R, Pessin J E, Rando T A, Richardson A, Schad!E E, Wyss-Coray T, Sierra F. Geroscience: Linking Aging to Chronic Disease. Cell. 2014; 159(4):709-13.
14. Dillin A, Hsu A L, Arantes-Oiiveira N, Lehrer-Graiwer J, Hsin H, Fraser A G, Kamath R S, Ahringer J, Kenyon C. Rates of behavior and aging specified by mitochondrial function during development. Science. 2002; 298(5602):2398-401.
15. Dirks A J, Leeuwenburgh C. Caloric restriction in humans: potential pitfalls and health concerns. Mechanisms of ageing and development. 2006; 127(1):1-7.
16. Taylor R C, Dillin A. Aging as an event of proteostasis collapse. Cold Spring Harbor perspectives in biology. 2011; 3(5).
17. Ciechanover A. Proteolysis: from the lysosome to ubiquitin and the proteasome. Nature reviews Molecular cell biology. 2005; 6(1):79-87.
18. Knecht E, Aguado C, Carcel J, Esteban I, Esteve J M, Ghislat G, Moruno J F, Vidal J M, Saez R. Intracellular protein degradation in mammalian cells: recent developments. Cellular and molecular life sciences: CMLS. 2009; 66(15):2427-43.
19. Kikis E A, Gidalevitz T, Morimoto Rl. Protein homeostasis in models of aging and age-related conformational disease. Advances in experimental medicine and biology. 2010; 694:138-59.
20. Powers E T, Morimoto Rl, Dillin A, Kelly J W, Balch W E. Biological and chemical approaches to diseases of proteostasis deficiency. Annual review of biochemistry. 2009; 78:959-91.
21. Gray D A, Woulfe J. Structural disorder and the loss of RNA homeostasis in aging and neurodegenerative disease. Frontiers in genetics. 2013; 4:149.
22. Ling S C, Polymenidou M, Cleveland D W. Converging mechanisms in ALS and FTD: disrupted RNA and protein homeostasis. Neuron. 2013; 79(3):416-38.
23. Lopez-Bigas N, Audit B, Ouzounis C, Parra G, Guigo R. Are splicing mutations the most frequent cause of hereditary disease? FEBS letters. 2005; 579(9):1900-3.
24. Wang G S, Cooper T A. Splicing in disease: disruption of the splicing code and the decoding machinery. Nature reviews Genetics. 2007; 8(10):749-61.
25. Andresen B S, Krainer A. When the genetic code is not enough—How sequence variations can affect pre-mRNA splicing and cause (complex) disease. In: A I-Chalabi A, Almasy K, editors. Genetics of complex human diseases: A laboratory manual. New York: CSHL press; 2009. p. 165-82.
26. Dredge B K, Polydorides A D, Darnell R B. The splice of life: alternative splicing and neurological disease. Nature reviews Neuroscience. 2001; 2(1):43-50.
27. Srebrow A, Kornblihtt A R. The connection between splicing and cancer. Journal of cell science. 2006; 119(Pt 13):2635-41.
28. Ward A J, Cooper T A. The pathobiology of splicing. The Journal of pathology. 2010; 220(2):152-63.
29. Huang L, Lou C H, Chan W, Shum E Y, Shao A, Stone E, Karam R, Song H W, Wilkinson M F. RNA homeostasis governed by cell type-specific and branched feedback loops acting on NMD. Molecular cell. 2011; 43(6):950-61.
30. Quidville V, Alsafadi S, Goubar A, Comma F, Scott V, Pioche-Durieu C, Girault I, Baconnais S, Le Cam E, Lazar V, Delaloge S, Saghatchian M, Pautier P, Morice P, Dessen P, Vagner S, Andre F. Targeting the deregulated spliceosome core machinery in cancer cells triggers mTOR blockade and autophagy. Cancer research. 2013; 73(7):2247-58.
31. Meshorer E, Soreq H. Pre-mRNA splicing modulations in senescence. Aging cell. 2002; 1(1):10-6.
32. Harries L W, Hernandez D, Henley W, Wood A R, Holly A C, Bradley-Smith R M, Yaghootkar H, Dutta A, Murray A, Frayling T M, Guralnik J M, Bandinelli S, Singleton A, Ferrucci L, Melzer D. Human aging is characterized by focused changes in gene expression and deregulation of alternative splicing. Aging cell. 2011; 10(5):868-78.
33. Tollervey J R, Wang Z, Hortobagyi T, Witten J T, Zarnack K, Kayikci M, Clark T A, Schweitzer A C, Rot G, Curk T, Zupan B, Rogelj B, Shaw C E, Ule J. Analysis of alternative splicing associated with aging and neurodegeneration in the human brain. Genome research. 2011; 21(10):1572-82.
34. Holly A C, Melzer D, Pilling L C, Fellows A C, Tanaka T, Ferrucci L, Harries L W. Changes in splicing factor expression are associated with advancing age in man. Mechanisms of ageing and development. 2013; 134(9):356-66.
35. Mazin P, Xiong J, Liu X, Van Z, Zhang X, Li M, He L, Somel M, Yuan Y, Phoebe Chen Y P, Li N, Hu Y, Fu N, Ning Z, Zeng R, Yang H, Chen W, Gelfand M, Khaitovich P. Widespread splicing changes in human brain development and aging. Molecular systems biology. 2013; 9:633.
36. Stout G J, Stigler E C, Essers P B, Mulder K W, Kolkman A, Snijders D S, van den Broek N J, Betist M C, Korswagen H C, Macinnes A W, Brenkman A B. lnsulin/IGF-1-mediated longevity is marked by reduced protein metabolism. Molecular systems biology. 2013; 9:679.
37. Venables J P, Klinck R, Koh C, Gervais-Bird J, Bramard A, lnkel L, Durand M, Couture S, Froehlich U, Lapointe E, Lucier J F, Thibault P, Rancourt C, Tremblay K, Prinos P, Chabot B, Elela S A. Cancer-associated regulation of alternative splicing. Nature structural & molecular biology. 2009; 16(6):670-6.
38. Douglas A G, Wood M J. RNA splicing: disease and therapy. Briefings in functional genomics. 2011; 10(3):151-64.
39. Lopez-Mejia I C, Vautrot V, De Toledo M, Behm-Ansmant I, Bourgeois C F, Navarro C L, Osorio F G, Freije J M, Stevenin J, De Sandre-Giovannoli A, Lopez-Olin C, Levy N, Branlant C, Tazi J. A conserved splicing mechanism of the LMNA gene controls premature aging. Human molecular genetics. 2011; 20(23):4540-55.
40. Consortium CeS. Genome sequence of the nematode C. elegans: a platform for investigating biology. Science. 1998; 282(5396):2012-8.
41. Hillier L W, Coulson A, Murray J1, Bao Z, Sulston J E, Waterston R H. Genomics in C. elegans: so many genes, such a little worm. Genome research. 2005; 15(12):1651-60.
42. Lai C H, Chou C Y, Ch′ang L Y, Liu C S, Lin W. Identification of novel human genes evolutionarily conserved in Caenorhabditis elegans by comparative proteomics. Genome research. 2000; 10(5):703-13.
43. Xu X, Kim S K. The early bird catches the worm: new technologies for the Caenorhabditis elegans toolkit. Nature reviews Genetics. 2011; 12(11):793-801.
44. Lund J, Tedesco P, Duke K, Wang J, Kim S K, Johnson T E. Transcriptional profile of aging in C. elegans. Current biology: C B. 2002; 12(18):1566-73.
45. Golden T R, Melov S. Microarray analysis of gene expression with age in individual nematodes. Aging cell. 2004; 3(3):111-24.
46. McCarroll S A, Murphy C T, Zou S, Pletcher S D, Chin C S, Jan Y N, Kenyon C, Bargmann Cl, Li H. Comparing genomic expression patterns across species identifies shared transcriptional profile in aging. Nature genetics. 2004; 36(2):197-204.
47. Ruzanov P, Riddle D L. Deep SAGE analysis of the Caenorhabditis elegans transcriptome. Nucleic acids research. 2010; 38(10):3252-62.
48. Heestand B N, Shen Y, Liu W, Magner D B, Storm N, Meharg C, Habermann B, Antebi A. Dietary Restriction Induced Longevity Is Mediated by Nuclear Receptor NHR-62 in Caenorhabditis elegans. PLoS genetics. 2013; 9(7):e1003651.
49. Kuroyanagi H, Kobayashi T, Milani S, Hagiwara M. Transgenic alternative-splicing reporters reveal tissue-specific expression profiles and regulation mechanisms in vivo. Nature methods. 2006; 3(11):909-15.
50. Kuroyanagi H, Ohno G, Sakane H, Maruoka H, Hagiwara M. Visualization and genetic analysis of alternative splicing regulation in vivo using fluorescence reporters in transgenic Caenorhabditis elegans. Nature protocols. 2010; 5(9):1495-517.
51. Wang E T, Sandberg R, Luo S, Khrebtukova I, Zhang L, Mayr C, Kingsmore S F, Schroth G P, Burge C B. Alternative isoform regulation in human tissue transcriptomes. Nature. 2008; 456(7221):470-6.
52. Ramani A K, Calarco J A, Pan 0, Mavandadi S, Wang Y, Nelson A C, Lee L J, Morris 0, Blencowe B J, Zhen M, Fraser A G. Genome-wide analysis of alternative splicing in Caenorhabditis elegans. Genome research. 2011; 21(2):342-8.
53. Barberan-Soler S, Zahler A M. Alternative splicing and the steady-state ratios of rnRNA isoforrns generated by it are under strong stabilizing selection in Caenorhabditis elegans. Molecular biology and evolution. 2008; 25(11):2431-7.
54. Barberan-Soler S, Medina P, Estella J, Williams J, Zahler A M. Co-regulation of alternative splicing by diverse splicing factors in Caenorhabditis elegans. Nucleic acids research. 2011; 39(2):666-74.
55. Barberan-Soler S, Zahler A M. Alternative splicing regulation during C. elegans development: splicing factors as regulated targets. PLoS genetics. 2008; 4(2):e1000001.
56. Thomas J, LeaK, Zucker-Aprison E, Blumenthal T. The spliceosornal snRNAs of Caenorhabditis elegans. Nucleic acids research. 1990; 18(9):2633-42.
57. Zahler A M. Pre-rnRNA splicing and its regulation in Caenorhabditis elegans. WorrnBook. 2012:1-21.
58. Morton J J, Blumenthal T. RNA processing in C. elegans. Methods in cell biology. 2011; 106:187-217.
59. Caudevilla C, Serra D, Miliar A, Codony C, Asins G, Bach M, Hegardt F G. Natural trans-splicing in carnitine octanoyltransferase pre-mRNAs in rat liver. Proceedings of the National Academy of Sciences of the United States of America. 1998; 95(21):12185-90.
60. Horiuchi T, Aigaki T. Alternative trans-splicing: a novel mode of pre-mRNA processing. Biology of the cell I under the auspices of the European Cell Biology Organization. 2006; 98(2):135-40.
61. Viles K D, Sullenger B A. Proximity-dependent and proximity-independent trans-splicing in mammalian cells. Rna. 2008; 14(6):1081-94.
62. Allen M A, Hillier L W, Waterston R H, Blumenthal T. A global analysis of C. elegans trans-splicing. Genome research. 2011; 21(2):255-64.
63. Lasda E L, Blumenthal T. Trans-splicing. Wiley interdisciplinary reviews RNA. 2011; 2(3):417-34.
64. Blumenthal T. Trans-splicing and operons in C. elegans. WormBook: the online review of C elegans biology. 2012:1-11.
65. Hu G J, Chen J, Zhao X N, Xu J J, Guo D O, Lu M, Zhu M, Xiong Y, Li 0, Chang C C, Song B L, Chang T V, Li B L. Production of ACAT1 56-kDa isoform in human cells via trans-splicing involving the ampicillin resistance gene. Cell research. 2013; 23(8):1007-24.
66. Calarco J A, Saltzman A L, 1p J Y, Blencowe B J. Technologies for the global discovery and analysis of alternative splicing. Advances in experimental medicine and biology. 2007; 623:64-84.
67. Hillier L W, Marth G T, Quinlan A R, Dooling D, Fewell G, Barnett D, Fox P, Glasscock Jl, Hickenbotham M, Huang W, Magrini V J, Riehl R J, Sander S N, Stewart D A, Stromberg M, Tsung E F, Wylie T, Schedl T, Wilson R K, Mardis E R. Whole-genome sequencing and variant discovery in C. elegans. Nature methods. 2008; 5(2):183-8.
68. Hillier L W, Reinke V, Green P, Hirst M, Marra M A, Waterston R H. Massively parallel sequencing of the polyadenylated transcriptome of C. elegans. Genome research. 2009; 19(4):657-66.
69. Spencer W C, Zeller G, Watson J D, Henz S R, Watkins K L, McWhirter R D, Petersen S, Sreedharan Vf, Widmer C, Jo J, Reinke V, Petrella L, Strome S, Von Stetina S E, Katz M, Shaham S, Raisch G, Miller D M, 3rd. A spatial and temporal map of C. elegans gene expression. Genome research. 2011; 21(2):325-41.
70. Sheives P, Lynch K W. Identification of cells deficient in signaling-induced alternative splicing by use of somatic cell genetics. Rna. 2002; 8(12):1473-81.
71. Ellis P D, Smith C W, Kemp P. Regulated tissue-specific alternative splicing of enhanced green fluorescent protein transgenes conferred by alpha-tropomyosin regulatory elements in transgenic mice. The Journal of biological chemistry. 2004; 279(35):36660-9.
72. Orengo J P, Bundman D, Cooper T A. A bichromatic fluorescent reporter for cell-based screens of alternative splicing. Nucleic acids research. 2006; 34(22):e148.
73. Bonano V I, Oltean S, Garcia-Blanco M A. A protocol for imaging alternative splicing regulation in vivo using fluorescence reporters in transgenic mice. Nature protocols. 2007; 2(9):2166-81.
74. Warzecha C C, Sato T K, Nabet B, Hogenesch J B, Carstens R P. ESRP1 and ESRP2 are epithelial cell-type-specific regulators of FGFR2 splicing. Molecular cell. 2009; 33(5):591-601.
75. Kuroyanagi H, Watanabe Y, Hagiwara M. CELF family RNA-binding protein UNC-75 regulates two sets of mutually exclusive exons of the unc-32 gene in neuron-specific manners in Caenorhabditis elegans. PLoS genetics. 2013; 9(2):e1003337.
76. MacMorris M, Brocker C, Blumenthal T. UAP56 levels affect viability and mRNA export in Caenorhabditis elegans. Rna. 2003; 9(7):847-57.
77. Lakowski B, Hekimi S. The genetics of caloric restriction in Caenorhabditis elegans. Proceedings of the National Academy of Sciences of the United States of America. 1998; 95(22):13091-6.
78. Ching T T, Paal A B, Mehta A, Zhong L, Hsu A L. drr-2 encodes an eiF4H that acts downstream of TOR in diet-restriction-induced longevity of C. elegans. Aging cell. 2010; 9(4):545-57.
79. Ching T T, Hsu A L. Solid plate-based dietary restriction in Caenorhabditis elegans. Journal of visualized experiments: JoVE. 2011(51). doi: 10.3791/2701.
80. McKay J P, Raizen D M, Gottschalk A, Schafer W R, Avery L. eat-2 and eat-18 are required for nicotinic neurotransmission in the Caenorhabditis elegans pharynx. Genetics. 2004; 166(1):161-9.
81. Trapnell C, Williams B A, Pertea G, Mortazavi A, Kwan G, van Baren M J, Salzberg S L, Wold B J, Pachter L. Transcript assembly and quantification by RNA-Seq reveals unannotated transcripts and isoform switching during cell differentiation. Nature biotechnology. 2010; 28(5):511-5.
82. Roberts A, Pimentel H, Trapnell C, Pachter L. Identification of novel transcripts in annotated genomes using RNA-Seq. Bioinformatics. 2011; 27(17):2325-9.
83. Anders S, Reyes A, Huber W. Detecting differential usage of exons from RNA-seq data. Genome research. 2012; 22(10):2008-17.
84. Pruitt K D, Brown G R, Hiatt S M, Thibaud-Nissen F, Astashyn A, Ermolaeva 0, Farrell C M, Hart J, Landrum M J, McGarvey K M, Murphy M R, O'Leary N A, Pujar S, Rajput B, Rangwala S H, Riddick L D, Shkeda A, Sun H, Tamez P, Tully R E, Wallin C, Webb D, Weber J, Wu W, DiCuccio M, Kitts P, Maglott D R, Murphy T D, Ostell J M. RefSeq: an update on mammalian reference sequences. Nucleic acids research. 2014; 42(Database issue):0756-63.
85. Cunningham F, Amode M R, Barrell D, Beal K, Billis K, BrentS, Carvalho-Silva D, Clapham P, Coates G, Fitzgerald S, GilL, Giron C G, Gordon L, Hourlier T, Hunt S E, Janacek S H, Johnson N, Juettemann T, Kahari A K, Keenan S, Martin F J, Maurel T, Mclaren W, Murphy D N, Nag R, Overduin B, Parker A, Patricio M, Perry E, Pignatelli M, RialHS, Sheppard D, Taylor K, Thormann A, Vullo A, Wilder S P, Zadissa A, Aken B L, Birney E, Harrow J, Kinsella R, Muffato M, Ruffier M, Searle S M, Spudich G, Trevanion S J, Yates A, Zerbino D R, Flicek P. Ensembl 2015. Nucleic acids research. 2015; 43(Databaseissue):0662-9.
86. Zarnack K, Konig J, Tajnik M, Martincorena I, Eustermann S, Stevant I, Reyes A, Anders S, Luscombe N M, Ule J. Direct competition between hnRNP C and U2AF65 protects the transcriptome from the exonization of Alu elements. Cell. 2013; 152(3):453-66.
87. Taveau M, Stockholm D, Spencer M, Richard I. Quantification of splice variants using molecular beacon or scorpion primers. Analytical biochemistry. 2002; 305(2):227-35. d
88. Kuroyanagi H, Ohno G, Milani S, Hagiwara M. The Fox-1 family and SUP-12 coordinately regulate tissue-specific alternative splicing in vivo. Molecular and cellular biology. 2007; 27(24):8612-21.
89. Ohno G, Hagiwara M, Kuroyanagi H. STAR family RNA-binding protein ASD-2 regulates developmental switching of mutually exclusive alternative splicing in vivo. Genes & development. 2008; 22(3):360-74.
90. Ohno G, Ono K, Togo M, Watanabe Y, Ono S, Hagiwara M, Kuroyanagi H. Muscle-specific splicing factors ASD-2 and SUP-12 cooperatively switch alternative pre-mRNA processing patterns of the ADF/cofilin gene in Caenorhabditis elegans. PLoS genetics. 2012; 8(10):e1002991.
91. Kuroyanagi H, Watanabe Y, Suzuki Y, Hagiwara M. Position-dependent and neuron-specific splicing regulation by the CELF family RNA-binding protein UNC-75 in Caenorhabditis elegans. Nucleic acids research. 2013; 41(7):4015-25.
92. Mair W, Morantte I, Rodrigues A P, Manning G, Montminy M, Shaw R J, Dillin A. Lifespan extension induced by AMPK and calcineurin is mediated by CRTC-1 and CREB. Nature. 2011; 470(7334):404-8. Epub 2011/02/19.
93. Burkewitz K, Morantte I, Weir H J, Yeo R, Zhang Y, Huynh F K, llkayeva 0, Hirschey M D, Grant A R, Mair W B. Neuronal CRTC-1 governs systemic mitochondrial metabolism and lifespan via a catecholamine signal. Cell 160, 842-55 (2015).
94. Li X, Zhao X, Fang Y, Jiang X, Duong T, Fan C, Huang C C, Kain S R. Generation of destabilized green fluorescent protein as a transcription reporter. The Journal of biological chemistry. 1998; 273(52):34970-5.
95. Fraser A G, Kamath R S, Zipperlen P, Martinez-Campos M, Sohrmann M, Ahringer J. Functional genomic analysis of C. elegans chromosome I by systematic RNA interference. Nature. 2000; 408(6810):325-30.
96. Kamath R S, Fraser A G, Dong Y, Poulin G, Durbin R, Golla M, Kanapin A, Le Bot N, Moreno S, Sohrmann M, Welchman D P, Zipperlen P, Ahringer J. Systematic functional analysis of the Caenorhabditis elegans genome using RNAi. Nature. 2003; 421(6920):231-7.
97. Longman D, Johnstone I L, Caceres J F. Functional characterization of S R and S R-related genes in Caenorhabditis elegans. The EMBO journal. 2000; 19(7):1625-37.
98. Kinnaird J H, Maitland K, Walker G A, Wheatley I, Thompson F J, Devaney E. HRP-2, a heterogeneous nuclear ribonucleoprotein, is essential for embryogenesis and oogenesis in Caenorhabditis elegans. Experimental cell research. 2004; 298(2):418-30.
99. Longman D, McGarvey T, McCracken S, Johnstone I L, Blencowe B J, Caceres J F. Multiple interactions between SRm160 and S R family proteins in enhancer-dependent splicing and development of C. elegans. Current biology: C B. 2001; 11(24):1923-33.
100. Dickinson D J, Ward J D, Reiner D J, Goldstein B. Engineering the Caenorhabditis elegans genome using Cas9-triggered homologous recombination. Nature methods. 2013; 10(10):1028-34.
101. Munding E M, Shiue L, Katzman S, Donohue J P, Ares M, Jr. Competition between pre-mRNAs for the splicing machinery drives global regulation of splicing. Molecular cell. 2013; 51(3):338-48.
102. Vellai T, Takacs-Vellai K, Zhang Y, Kovacs A L, Orosz L, Muller F. Genetics: influence of TOR kinase on lifespan in C. elegans. Nature. 2003; 426(6967):620. Epub 2003/12/12.
103. Jia K, Chen D, Riddle D L. The TOR pathway interacts with the insulin signaling pathway to regulate C. elegans larval development, metabolism and life span. Development. 2004; 131(16):3897-906.
104. Robida-Stubbs S, Glover-Cutter K, Lamming D W, Mizunuma M, Narasimhan S D, Neumann-Haefelin E, Sabatini D M, Blackwell T K. TOR signaling and rapamycin influence longevity by regulating SKN-1/Nrf and DAF-16/FoxO. Cell metabolism. 2012; 15(5):713-24.
105. Duvel K, Yecies J L, Menon S, Raman P, Lipovsky A I, Souza A L, Triantafellow E, Ma 0, Gorski R, Cleaver S, Vander Heiden M G, MacKeigan J P, Finan P M, Clish C B, Murphy L O, Manning B D. Activation of a metabolic gene regulatory network downstream of mTOR complex 1. Molecular cell. 2010; 39(2):171-83.
106. Corioni M, Antih N, Tanackovic G, Zavolan M, Kramer A. Analysis of in situ pre-mRNA targets of human splicing factor SF1 reveals a function in alternative splicing. Nucleic acids research. 2011; 39(5):1868-79.
107. Zhou Z, Sim J, Griffith J, Reed R. Purification and electron microscopic visualization of functional human spliceosomes. Proceedings of the National Academy of Sciences of the United States of America. 2002; 99(19):12203-7.
108. Curran S P, Ruvkun G. Lifespan regulation by evolutionarily conserved genes essential for viability. PLoS genetics. 2007; 3(4):e56.
109. Vandenbroucke, I I, Vandesompele J, Paepe A D, Messiaen L. Quantification of splice variants using real-time PCR. Nucleic acids research. 2001; 29(13):E68-8.
110. Riedel C G, Dowen R H, Lourenco G F, Kirienko N V, Heimbucher T, West J A, Bowman S K, Kingston R E, Dillin A, Asara J M, Ruvkun G. DAF-16 employs the chromatin remodeller SWI/SNF to promote stress resistance and longevity. Nature cell biology. 2013; 15(5):491-501.
111. Goedert M. Neurodegenerative tauopathy in the worm. Proceedings of the National Academy of Sciences of the United States of America. 2003; 100(17):9653-5.
112. Kraemer B C, Zhang B, Leverenz J B, Thomas J H, Trojanowski J Q, Schellenberg G D. Neurodegeneration and defective neurotransmission in a Caenorhabditis elegans model of tauopathy. Proceedings of the National Academy of Sciences of the United States of America. 2003; 100(17):9980-5.
113. Zid B M, Rogers A N, Katewa S D, Vargas M A, Kolipinski M C, Lu T A, Benzer S, Kapahi P. 4E-B P extends lifespan upon dietary restriction by enhancing mitochondrial activity in Drosophila. Cell. 2009; 139(1):149-60.
114. Laplante, M. & Sabatini, D. mTOR Signaling in Growth Control and Disease. Cell 149, (2012).
115. Ma, L., Tan, Z., Teng, Y., Hoersch, S. & Horvitz, R. In vivo effects on intron retention and exon skipping by the U2A F large subunit and SF1/BBP in the nematode Caenorhabditis elegans. RNA 17, 2201-2211 (2011).

1. A method for determining the biological age of a eukaryotic cell, the method comprising:
detecting a signature of splicing events in the eukaryotic cell; and
determining the biological age of the eukaryotic cell by comparing the signature to one or more control signatures of defined age;
wherein the detecting comprises RNA-Seq technology.

2. The method of claim 1, further comprising isolating nucleic acid from the eukaryotic cell.

3. The method of claim 1, further comprising obtaining the eukaryotic cell from an animal.

4. The method of claim 3, wherein the animal is a mammal.

5. The method of claim 4, wherein the mammal is a human.

6-31. (canceled)

32. A method for identifying one or more biomarkers of aging, the method comprising:
(a) comparing: (i) a first signature of splicing events in one or more cells from a first animal, (ii) a second signature of splicing events in one or more cells from a chronologically older animal of the same species; and a third signature of splicing events in one or more cells from a third animal that has been calorically restricted, wherein the first animal, the second animal, and the third animal are all of the same species, and
(b) identifying one or more splicing event variations between the first signature and the second signature that are also splicing event variations between the first signature and the third signature;
wherein the first animal and the third animal are the same chronological age.

33. (canceled)

34. The method of claim 32, further comprising determining the first signature, the second signature, the third signature, the first and second signature, the first and third signature, the second and third signature, or the first, second, and third signature.

35. The method of claim 32, wherein the animal is a mammal.

36. The method of claim 35, wherein the mammal is a human.

37-38. (canceled)

39. A method for determining whether a subject is at an increased risk for developing an age-related disorder, the method comprising:
detecting a spliceosome signature comprising information on the presence, or expression level, of two or more components of the spliceosome complex in the eukaryotic cell; and
determining whether the subject is at an increased risk for developing an age-related disorder by comparing the signature to one or more control signatures of defined age.

40. (canceled)

41. The method of claim 38 or 39, wherein the age-related disorder is a bone loss disorder.

42. The method of claim 38 or 39, wherein the age-related disorder is a neuromuscular disorder.

43. The method of claim 38 or 39, wherein the age-related disorder is a neurodegenerative disorder or a cognitive disorder.

44. The method of claim 38 or 39, wherein the age-related disorder is a metabolic disorder.

45. The method of claim 38 or 39, wherein the age-related disorder is sarcopenia, osteoarthritis, chronic fatigue syndrome, Alzheimer's disease, senile dementia, mild cognitive impairment due to aging, schizophrenia, Parkinson's disease, Huntington's disease, Pick's disease, Creutzfeldt-Jakob disease, stroke, CNS cerebral senility, age-related cognitive decline, pre-diabetes, diabetes, obesity, osteoporosis, coronary artery disease, cerebrovascular disease, heart attack, stroke, peripheral arterial disease, aortic valve disease, stroke, mild cognitive impairment, pre-dementia, dementia, macular degeneration, or cataracts.

46-71. (canceled)

72. A transgenic non-human animal comprising a plurality of cells comprising at least three different nucleic acids, wherein each nucleic acid encodes a different protein whose expression requires at least one specific splicing event in the cells.

73. The transgenic non-human animal of claim 72, wherein the protein is a fluorescent protein.

74. The transgenic non-human animal of claim 72, wherein the protein is detectably-labeled.

75. The transgenic non-human animal of claim 74, wherein the detectable label is an epitope tag.

76. (canceled)

77. The transgenic non-human animal of claim 72, wherein the animal is a fish.

78-88. (canceled)
